(19) United States
(12) Patent Application Publication
(10) Pub. No.: US 2024/0384350 A1
(43) Pub. Date: Nov. 21, 2024

(54) METHODS OF TREATING CANCER WITH KINASE INHIBITORS

(71) Applicant: BOARD OF REGENTS, THE UNIVERSITY OF TEXAS SYSTEM, Austin, TX (US)

(72) Inventors: Jacqulyne P. ROBICHAUX, Houston, TX (US); John V. HEYMACH, Houston, TX (US); Simon HEEKE, Houston, TX (US)

(73) Assignee: BOARD OF REGENTS, THE UNIVERSITY OF TEXAS SYSTEM, Austin, TX (US)

(21) Appl. No.: 18/263,341

(22) PCT Filed: Jan. 28, 2022
(86) PCT No.: PCT/US2022/014367

§ 371 Date: Jul. 27, 2023

Related U.S. Application Data
(60) Provisional application No. 63/143,710, filed on Jan. 29, 2021.
(60) Provisional application No. 63/244,169, filed on Sep. 14, 2021.

(51) Int. Cl. C12Q 1/6886; A61K 31/404; A61K 31/4709; A61K 31/496; A61K 31/497; A61K 31/505; A61K 31/506; A61K 31/517; A61K 31/519; A61K 31/5377; A61K 31/55; A61K 31/553

(52) U.S. Cl. CPC C12Q 1/6886; A61K 31/404; A61K 31/4709; A61K 31/496; A61K 31/497; A61K 31/505; A61K 31/506; A61K 31/517; A61K 31/519; A61K 31/5377; A61K 31/55; A61K 31/553; C12Q 2600/106; C12Q 2600/156

(57) ABSTRACT

Aspects of the present disclosure are directed to methods for treating a subject having cancer. Certain aspects relate to treating a subject for lung cancer by administering a kinase inhibitor to a subject determined, from analysis of tumor DNA from the subject, to have an EGFR mutation of a particular classification. Further aspects relate to methods for treating a subject for lung cancer by detecting an EGFR mutation of a particular classification in tumor DNA from the subject and administering an effective amount of a kinase inhibitor to the subject. Also disclosed are methods for stratifying and prognosing subjects based on EGFR mutation classification.

This application claims benefit of priority of U.S. Provisional Application No. 63/143,710, filed Jan. 29, 2021 and U.S. Provisional Application No. 63/244,169, filed Sep. 14, 2021, which are hereby incorporated by reference in their entirety.

This invention was made with government support under R01CA247975 awarded by the National Institutes of Health. The government has certain rights in the invention.

### BACKGROUND

### I. Field of the Disclosure

Aspects of this disclosure relate, generally, to at least the fields of cancer biology, molecular biology, and medicine.

### II. Background

Epidermal Growth Factor Receptor (EGFR) mutations are established driver mutations in non-small cell lung cancer (NSCLC), and targeted therapies are approved for patients with select EGFR mutations. However, there are additional EGFR mutations for which effective therapies have yet to be identified, and the frequency and impact of atypical EGFR mutations on drug sensitivity are unknown.

Mutations in epidermal growth factor (EGFR) occur in 10-15% of patients with non-small cell lung cancer (NSCLC) and can be divided into classical or atypical mutations¹⁻⁴. Classical EGFR mutations include L858R and exon 19 deletions (Ex19del), and patients with these mutations have marked improvements in clinical endpoints when treated with first-, second-, and third-generation TKIs⁵⁻⁷. The current standard of care for patients with classical EGFR mutant NSCLC is treatment with the third-generation TKI osimertinib⁸. The Phase III study of osimertinib resulted in an objective response rate (ORR) of 80%, a median progression free survival (mPFS) of 18.9 months⁷, and a median overall survival (mOS) of 38.6⁹ months in treatment-naïve patients, a significant improvement in clinical outcomes compared to earlier generations of EGFR TKIs.

Other EGFR mutations in the kinase domain (exon 18-21) have also been established as oncogene drivers for NSCLC, however, the therapeutic choices for those NSCLCs are limited. Patients with atypical EGFR mutations have experienced heterogeneous and reduced responses to EGFR inhibitors^{1,3,4,10-15}. In a Phase II study (KCSG-LU15-09) of treatment-naïve patients harboring atypical EGFR mutations, osimertinib treatment lead to an ORR of 50% and a mPFS of 8.2 months¹⁶, and studies of acquired osimertinib-resistance have shown acquisition of atypical mutations in exons 18¹⁷⁻²² and 20²³⁻²⁸. Currently, the only atypical EGFR mutations with an FDA-approved treatment are EGFR S768I, L861Q, and G719X, for which afatinib deemed effective based on retrospective studies²⁹⁻³². Atypical EGFR mutations without an FDA-approved TKI are often viewed as one entity, and there are no clear established guidelines for EGFR TKI treatment for patients with these mutations, resulting in patients often receiving cytotoxic chemotherapy. Clinical trial design and treatment of patients with atypical EGFR mutations has often relied on the exonic location of the mutations to predict treatment, although heterogeneity in drug sensitivity across a single exon has been clearly observed^{1,11,12,33-36}.

Recognized is a need for a system for identifying and classifying EGFR mutations that is predictive of response to cancer therapy for treatment of patients and for clinical trial design, as well as methods of using such a system for predicting the efficacy of cancer therapies to more effectively treat cancer.

### SUMMARY

Aspects of the present disclosure address certain needs in the art by providing methods for treating a subject with cancer (e.g., lung cancer) and methods for predicting patient response to a cancer therapy. Accordingly, provided herein, in some aspects, are methods for treating a subject for cancer, e.g., lung cancer, the method comprising administering an effective amount of one or more kinase inhibitors from one or more kinase classes to a subject determined, from analysis of tumor DNA from the subject, to have one or more EGFR mutations. Also disclosed are methods for treating a subject for cancer, e.g., lung cancer, the method comprising: (a) detecting one or more EGFR mutations in tumor DNA from the subject; and (b) administering an effective amount of one or more kinase inhibitors from one or more kinase classes depending on the detected EGFR mutations. In some embodiments, the cancer is lung cancer. In some embodiments, the cancer is non-small cell lung cancer. In some embodiments, the EGFR mutation is a classical-like mutation, an exon 20 near-loop insertion mutation (Ex20ins-NL), an exon 20 far-loop insertion mutation (Ex20ins-FL), a T790M-like-sensitive (T790M-like-3S) mutation, a T790M-like-resistant (T790M-like-3R) mutation, or a P-loop and αC-helix compressing (PACC) mutation.

Embodiments of the disclosure include methods for treating a subject having cancer, methods for improving the efficacy of kinase inhibitors used to treat a subject having cancer, methods for identifying a subject with cancer as a candidate for a treatment with a particular kinase inhibitor, methods for identification of an EGFR mutation, methods for classification of one or more EGFR mutations, and methods and compositions for treating a subject having a lung cancer. Methods of the disclosure can include 1, 2, 3, 4, 5, 6, or more of the following steps: determining a subject to have cancer, providing a one or more kinase inhibitors to a subject, providing an EGFR inhibitor to a subject, providing an alternative therapy to a subject, providing two or more types of cancer therapy to a subject, identifying one or more kinase inhibitors as being in need of improved efficacy, detecting one or more EGFR mutations in tumor DNA from a subject, identifying a subject as being a candidate for treatment with one or more particular kinase inhibitors, identifying a subject as being sensitive to one or more particular kinase inhibitors, identifying a subject as being resistant to one or more particular kinase inhibitors. Certain embodiments of the disclosure may exclude one or more of the preceding elements and/or steps.

Disclosed herein, in some aspects, is a method for classifying a cancer sample, the method comprising (a) detecting an EGFR mutation in the cancer sample; and (b) classifying the cancer sample as: (i) a classical-like EGFR mutant cancer, wherein the EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M; (ii) a T790M-like-3S EGFR mutant cancer, wherein the EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M; (iii) a T790M-like-3R EGFR mutant cancer, wherein the EGFR mutation is Ex19del T790M C797S, Ex19del T790M L792H, G724S T790M, L718Q T790M, L858R T790M C797S, or L858R T790M L718Q; (iv) an Exon20ins-NL EGFR mutant cancer, wherein the EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP; (v) an Exon20ins-FL EGFR mutant cancer, wherein the EGFR mutation is H773_V774 insNPH, H773_V774 insAH, H773dupH, V774_C775 insHV, V774_C775 insPR; or (vi) a P-loop αC-helix compressing EGFR mutant cancer, wherein the EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774. Any one or more of the preceding EGFR mutations may be excluded from embodiments of the disclosure. In some embodiments, the method further comprises classifying the cancer sample as sensitive to an EGFR inhibitor. In some embodiments, the EGFR inhibitor is a first-generation EGFR inhibitor, a second-generation EGFR inhibitor, a third generation EGFR inhibitor, or an EGFR inhibitor specific to mutations associated with EGFR exon 20. In some embodiments, the method further comprises classifying the cancer sample as insensitive to an EGFR inhibitor. In some embodiments, the EGFR inhibitor is a first-generation EGFR inhibitor, a second-generation EGFR inhibitor, a third generation EGFR inhibitor, or an EGFR inhibitor specific to mutations associated with EGFR exon 20.

In some embodiments, the method further comprises classifying the cancer sample as sensitive to a first-generation EGFR inhibitor, a second-generation EGFR inhibitor, a third generation EGFR inhibitor, or an EGFR inhibitor specific to mutations associated with EGFR exon 20 if the cancer sample is classified as a classical-like EGFR mutant cancer sample. In some embodiments, the method further comprises classifying the cancer sample as (i) sensitive to a third generation EGFR inhibitor; and (ii) insensitive to a first-generation EGFR inhibitor or a second-generation EGFR inhibitor if the cancer sample is classified as a T790M-like-3S EGFR mutant cancer. In some embodiments, the method further comprises classifying the cancer sample as insensitive to a first-generation EGFR inhibitor, a second-generation EGFR inhibitor, or a third generation EGFR inhibitor if the cancer sample is classified as a T790M-like-3R EGFR mutant cancer. In some embodiments, the method further comprises classifying the cancer sample as (i) sensitive to a second-generation EGFR inhibitor or an EGFR inhibitor specific to mutations associated with EGFR exon 20 and (ii) insensitive to a third-generation EGFR inhibitor if the cancer sample is classified as a Exon20ins-NL EGFR mutant cancer. In some embodiments, the method further comprises classifying the cancer sample as insensitive to a first-generation EGFR inhibitor, a second-generation EGFR inhibitor, and a third generation EGFR inhibitor if the cancer sample is classified as a Exon20ins-FL EGFR mutant cancer. In some embodiments, the method further comprises classifying the cancer sample as (i) sensitive to a second-generation EGFR inhibitor and (ii) insensitive to a third generation EGFR inhibitor if the cancer sample is classified as a P-loop αC-helix compressing EGFR mutant cancer.

Disclosed herein, in some aspects, is a method for classifying a cancer sample, the method comprising (a) detecting an EGFR mutation in the cancer sample, wherein the EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M; and (b) classifying the cancer sample as a T790M-like-3S mutant cancer. In some embodiments, the method further comprises classifying the cancer sample as sensitive to an EGFR inhibitor. In some embodiments, the EGFR inhibitor is a third generation EGFR inhibitor. In some embodiments, the method further comprises classifying the cancer sample as insensitive to an EGFR inhibitor. In some embodiments, the EGFR inhibitor is a first-generation EGFR inhibitor or a second-generation EGFR inhibitor.

Disclosed herein, in some aspects, is a method for classifying a cancer sample, the method comprising (a) detecting an EGFR mutation in the cancer sample, wherein the EGFR mutation is Ex19del T790M C797S, Ex19del T790M L792H, G724S T790M, L718Q T790M, L858R T790M C797S, or L858R T790M L718Q; and (b) classifying the cancer sample as a T790M-like-3R EGFR mutant cancer. In some embodiments, the method further comprises classifying the cancer sample as insensitive to an EGFR inhibitor. In some embodiments, the EGFR inhibitor is a first-generation EGFR inhibitor, a second-generation EGFR inhibitor, or a third generation EGFR inhibitor.

Disclosed herein, in some aspects, is a method for classifying a cancer sample, the method comprising (a) detecting an EGFR mutation in the cancer sample, wherein the EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP; and (b) classifying the cancer sample as a exon 20 near-loop insertion EGFR mutant cancer. In some embodiments, the method further comprises classifying the cancer sample as sensitive to an EGFR inhibitor. In some embodiments, the EGFR inhibitor is a second-generation EGFR inhibitor or an EGFR inhibitor specific to mutations associated with EGFR exon 20. In some embodiments, the method further comprises classifying the cancer sample as insensitive to an EGFR inhibitor. In some embodiments, the EGFR inhibitor is a first-generation EGFR or a third-generation EGFR inhibitor.

Disclosed herein, in some aspects, is a method for classifying a cancer sample, the method comprising (a) detecting an EGFR mutation in the cancer sample, wherein the EGFR mutation is H773_V774 insNPH, H773_V774 insAH, H773dupH, V774_C775 insHV, V774_C775 insPR; and (b) classifying the cancer sample as a exon 20 far-loop insertion EGFR mutant cancer. In some embodiments, the method further comprises classifying the cancer sample as insensitive to an EGFR inhibitor. In some embodiments, the EGFR inhibitor is a first-generation EGFR inhibitor, a second-generation EGFR inhibitor, or a third generation EGFR inhibitor.

Disclosed herein, in some aspects, is a method for classifying a cancer sample, the method comprises (a) detecting an EGFR mutation in the cancer sample, wherein the EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M; and (b) classifying the cancer sample as a P-loop αC-helix compressing EGFR mutant cancer. In some embodiments, the method further comprises classifying the cancer sample as sensitive to a EGFR inhibitor. In some embodiments, the EGFR inhibitor is a second-generation EGFR inhibitor. In some embodiments, the method further comprises classifying the cancer sample as insensitive to an EGFR inhibitor. In some embodiments, the EGFR inhibitor is a third generation EGFR inhibitor.

In some embodiments, the method further comprises, prior to (a), obtaining the cancer sample from a subject. In some embodiments, the method further comprises extracting tumor DNA from the cancer sample. In some embodiments, (a) comprises sequencing tumor DNA from the cancer sample. In some embodiments, the cancer sample is a lung cancer sample. In some embodiments, the lung cancer sample is a non-small cell lung cancer sample.

Disclosed herein, in some aspects, is a method for treating a subject for cancer, the method comprising administering an effective amount of an EGFR inhibitor to a subject determined, from analysis of tumor DNA from the subject, to have a classical-like EGFR mutation. In some embodiments, the EGFR inhibitor is a first-generation EGFR inhibitor, a second-generation EGFR inhibitor, a third generation EGFR inhibitor, or an EGFR inhibitor specific to mutations associated with EGFR exon 20. In some embodiments, the EGFR inhibitor is Erlotinib, Geftinib, AZD3759, Sapatinib, Lapatinib, Tucatinib, Afatinib, Dacomitinib, Neratinib, Tarlox-TKI, Tarloxotinib, Osimertinib, Nazartinib, Olmutinib, Rocelitinib, Naquotinib, Lazertinib, TAS 6417, AZ5104, or TAK-788 (mobocertinib). In some embodiments, the classical-like EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M.

Disclosed herein, in some aspects, is a method for treating a subject for cancer, the method comprising administering an effective amount of an EGFR inhibitor to a subject determined, from analysis of tumor DNA from the subject, to have a T790M-like-3S EGFR mutation. In some embodiments, the EGFR inhibitor is a third generation EGFR inhibitor. In some embodiments, the EGFR inhibitor is Osimertinib, Nazartinib, Olmutinib, Rocelitinib, Naquotinib, or Lazertinib. In some embodiments, the EGFR inhibitor is not a first-generation EGFR inhibitor or a second-generation EGFR inhibitor. In some embodiments, the EGFR inhibitor is not Erlotinib, Geftinib, AZD3759, Sapatinib, Lapatinib, Tucatinib, Afatinib, Dacomitinib, Neratinib, Tarlox-TKI, or Tarloxotinib. In some embodiments, the T790M-like-3S EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M.

Disclosed herein, in some aspects, is a method for treating a subject for cancer, the method comprising administering an effective amount of a tyrosine kinase inhibitor to a subject determined, from analysis of tumor DNA from the subject, to have a T790M-like-3R EGFR mutation, wherein the tyrosine kinase inhibitor is not an EGFR inhibitor. In some embodiments, the tyrosine kinase inhibitor is a PKC inhibitor. In some embodiments, the PKC inhibitor is Ruboxistaurin, Midostaurin, Sotrastaurin, Chelerythrine, Miyabenol C, Myricitrin, Gossypol, Verbascoside, BIM-1, Bryostatin 1, or Tamoxifen. In some embodiments, the tyrosine kinase inhibitor is an ALK inhibitor. In some embodiments, the ALK inhibitor is AZD3463, Brigatinib, Crizotinib, Ceritinib, Alectinib, Lorlatinib, Ensartinib, Entrectinib, Repotrectinib, Belizatinib, Alkotinib, Foritinib, CEP-37440, TQ-B3139, PLB1003, TPX-0131, or ASP-3026. In some embodiments, the T790M-like-3R EGFR mutation is Ex19del T790M C797S, Ex19del T790M L792H, G724S T790M, L718Q T790M, L858R T790M C797S, or L858R T790M L718Q.

Disclosed herein, in some aspects, is a method for treating a subject for cancer, the method comprising administering an effective amount of an EGFR inhibitor to a subject determined, from analysis of tumor DNA from the subject, to have a exon 20 near-loop insertion EGFR mutation. In some embodiments, the EGFR inhibitor is a second-generation EGFR inhibitor or an EGFR inhibitor specific to mutations associated with EGFR exon 20. In some embodiments, the EGFR inhibitor is Afatinib, Dacomitinib, Neratinib, Tarlox-TKI, Tarloxotinib, TAS 6417, AZ5104, or TAK-788 (mobocertinib). In some embodiments, the EGFR inhibitor is not a first-generation EGFR inhibitor or a third generation EGFR inhibitor. In some embodiments, the EGFR inhibitor is not Erlotinib, Geftinib, AZD3759, Sapatinib, Lapatinib, Tucatinib, Osimertinib, Nazartinib, Olmutinib, Rocelitinib, Naquotinib, or Lazertinib. In some embodiments, the exon 20 near-loop insertion EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP.

Disclosed herein, in some aspects, is a method for treating a subject for cancer, the method comprising administering an effective amount of an EGFR inhibitor to a subject determined, from analysis of tumor DNA from the subject, to have a exon 20 far-loop insertion EGFR mutation. In some embodiments, the EGFR inhibitor is an EGFR inhibitor specific to mutations associated with EGFR exon 20. In some embodiments, the EGFR inhibitor is TAS 6417, AZ5104, or TAK-788 (mobocertinib). In some embodiments, the exon 20 far-loop insertion EGFR mutation is H773_V774 insNPH, H773_V774 insAH, H773dupH, V774_C775 insHV, V774_C775 insPR.

Disclosed herein, in some aspects, is a method for treating a subject for cancer, the method comprising administering an effective amount of an EGFR inhibitor to a subject determined, from analysis of tumor DNA from the subject, to have a P-loop αC-helix compressing EGFR mutation. In some embodiments, the EGFR inhibitor is a first-generation EGFR inhibitor, a second-generation EGFR inhibitor, or an EGFR inhibitor specific to mutations associated with EGFR exon 20. In some embodiments, the EGFR inhibitor is Erlotinib, Geftinib, AZD3759, Sapatinib, Lapatinib, Tucatinib, Afatinib, Dacomitinib, Neratinib, Tarlox-TKI, Tarloxotinib, TAS 6417, AZ5104, or TAK-788 (mobocertinib). In some embodiments, the EGFR inhibitor is a second-generation EGFR inhibitor. In some embodiments, the EGFR inhibitor is Afatinib, Dacomitinib, Neratinib, Tarlox-TKI, or Tarloxotinib. In some embodiments, the EGFR inhibitor is not a second-generation EGFR inhibitor. In some embodiments, the EGFR inhibitor is not Afatinib, Dacomitinib, Neratinib, Tarlox-TKI, or Tarloxotinib. In some embodiments, the P-loop αC-helix compressing EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M.

In some embodiments, the subject has lung cancer, In some embodiments, the subject has non-small cell lung cancer. In some embodiments, the subject does not have lung cancer.

Disclosed herein, in some aspects, is a method for treating a subject for lung cancer, the method comprising administering an effective amount of osimertinib, nazartinib, olmutinib, rocelitinib, naquotinib, lazertinib, or a combination thereof to a subject determined, from analysis of tumor DNA from the subject, to have an EGFR mutation, wherein the EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M.

Also disclosed herein, in some aspects, is method for treating a subject for lung cancer, the method comprising: (a) detecting a EGFR mutation in tumor DNA from the subject, wherein the EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M; and (b) administering an effective amount of osimertinib, nazartinib, olmutinib, rocelitinib, naquotinib, lazertinib, or a combination thereof to the subject.

In some embodiments, the method comprises administering osimertinib to the subject. In some embodiments, the method comprises administering nazartinib to the subject. In some embodiments, the method comprises administering olmutinib to the subject. In some embodiments, the method comprises administering rocelitinib to the subject. In some embodiments, the method comprises administering naquotinib to the subject. In some embodiments, the method comprises administering lazertinib to the subject.

In some embodiments, the EGFR mutation is Ex19del T790M. In some embodiments, the EGFR mutation is Ex19del T790M L718V. In some embodiments, the EGFR mutation is Ex19del T790M G724S. In some embodiments, the EGFR mutation is G719A T790M. In some embodiments, the EGFR mutation is G719S T790M. In some embodiments, the EGFR mutation is H773R T790M. In some embodiments, the EGFR mutation is I744_E749del insMKK. In some embodiments, the EGFR mutation is L747_K754 delinsATSPE. In some embodiments, the EGFR mutation is L858R T790M L792H. In some embodiments, the EGFR mutation is L858R T790M V843I. In some embodiments, the EGFR mutation is L858R T790M. In some embodiments, the EGFR mutation is S768I T790M. In some embodiments, the EGFR mutation is T790M.

In some embodiments, the subject was previously treated with a cancer therapy. In some embodiments, the cancer therapy comprised erlotinib, gefitinib, AZD3759, or sapatinib. In some embodiments, the cancer therapy comprised chemotherapy. In some embodiments, the subject was determined to be resistant to the cancer therapy.

Disclosed herein, in some aspects, is a method for treating a subject for lung cancer, the method comprising administering an effective amount of afatinib, dacomitinib, neratinib, tarlox-TKI, tarloxotinib, TAS6417 (CLN-081), AZ5104, TAK-788 (mobocertinib), or a combination thereof to a subject determined, from analysis of tumor DNA from the subject, to have an EGFR mutation, wherein the EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP.

Also disclosed herein, in some aspects, is a method for treating a subject for lung cancer, the method comprising: (a) detecting an EGFR mutation in tumor DNA from the subject, wherein the EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP; and (b) administering an effective amount of afatinib, dacomitinib, neratinib, tarlox-TKI, tarloxotinib, TAS6417 (CLN-081), AZ5104, TAK-788 (mobocertinib), or a combination thereof to the subject.

In some embodiments, the method comprises administering afatinib to the subject. In some embodiments, the method comprises administering dacomitinib to the subject. In some embodiments, the method comprises administering neratinib to the subject. In some embodiments, the method comprises administering tarlox-TKI to the subject. In some embodiments, the method comprises administering tarloxotinib to the subject. In some embodiments, the method comprises administering TAS6417 (CLN-081) to the subject. In some embodiments, the method comprises administering AZ5104 to the subject. In some embodiments, the method comprises administering TAK-788 (mobocertinib) to the subject.

In some embodiments, the EGFR mutation is A767_V769dupASV. In some embodiments, the EGFR mutation is A767_S768insTLA. In some embodiments, the EGFR mutation is S768_D770dupSVD. In some embodiments, the EGFR mutation is S768_D770dupSVD L858Q. In some embodiments, the EGFR mutation is S768_D770dupSVD R958H. In some embodiments, the EGFR mutation is S768_D770dupSVD V769M. In some embodiments, the EGFR mutation is V769_D770insASV. In some embodiments, the EGFR mutation is V769_D770insGSV. In some embodiments, the EGFR mutation is V769_D770insGVV. In some embodiments, the EGFR mutation is V769_D770insMASVD. In some embodiments, the EGFR mutation is D770_N771insNPG. In some embodiments, the EGFR mutation is D770_N771insSVD. In some embodiments, the EGFR mutation is D770del insGY. In some embodiments, the EGFR mutation is D770_N771 insG. In some embodiments, the EGFR mutation is D770_N771 insY H773Y. In some embodiments, the EGFR mutation is N771dupN. In some embodiments, the EGFR mutation is N771dupN G724S. In some embodiments, the EGFR mutation is N771_P772insHH. In some embodiments, the EGFR mutation is N771_P772insSVDNR. In some embodiments, the EGFR mutation is P772_H773insDNP.

In some embodiments, the subject was previously treated with a cancer therapy. In some embodiments, the cancer therapy comprised erlotinib, gefitinib, AZD3759, or sapatinib. In some embodiments, the cancer therapy comprised osimertinib, nazartinib, olmutinib, rocelitinib, naquotinib, lazertinib. In some embodiments, the cancer therapy comprised chemotherapy. In some embodiments, the subject was determined to be resistant to the cancer therapy.

Disclosed herein, in some aspects, is a method for treating a subject for lung cancer, the method comprising administering an effective amount of afatinib, dacomitinib, neratinib, tarlox-TKI, tarloxotinib, or a combination thereof to a subject determined, from analysis of tumor DNA from the subject, to have an EGFR mutation, wherein the EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M.

Also disclosed herein, in some aspects, is a method for treating a subject for lung cancer, the method comprising: (a) detecting an EGFR mutation in tumor DNA from the subject, wherein the EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M; and (b) administering an effective amount of afatinib, dacomitinib, neratinib, tarlox-TKI, tarloxotinib, or a combination thereof to the subject.

In some embodiments, the method comprises administering afatinib to the subject. In some embodiments, the method comprises administering dacomitinib to the subject. In some embodiments, the method comprises administering neratinib to the subject. In some embodiments, the method comprises administering tarlox-TKI to the subject. In some embodiments, the method comprises administering tarloxotinib to the subject.

In some embodiments, the EGFR mutation is A750_I759del insPN. In some embodiments, the EGFR mutation is E709_T710del insD. In some embodiments, the EGFR mutation is E709A. In some embodiments, the EGFR mutation is E709A G719A. In some embodiments, the EGFR mutation is E709A G719S. In some embodiments, the EGFR mutation is E709K. In some embodiments, the EGFR mutation is E709K G719S. In some embodiments, the EGFR mutation is E736K. In some embodiments, the EGFR mutation is E746_A750del A647T. In some embodiments, the EGFR mutation is E746_A750del R675W. In some embodiments, the EGFR mutation is E746_T751del insV S768C. In some embodiments, the EGFR mutation is Ex19del C797S. In some embodiments, the EGFR mutation is Ex19del G796S. In some embodiments, the EGFR mutation is Ex19del L792H. In some embodiments, the EGFR mutation is Ex19del T854I. In some embodiments, the EGFR mutation is G719A. In some embodiments, the EGFR mutation is G719A D761Y. In some embodiments, the EGFR mutation is G719A L861Q. In some embodiments, the EGFR mutation is G719A R776C. In some embodiments, the EGFR mutation is G719A S768I. In some embodiments, the EGFR mutation is G719C S768I. In some embodiments, the EGFR mutation is G719S. In some embodiments, the EGFR mutation is G719S L861Q. In some embodiments, the EGFR mutation is G719S S768I. In some embodiments, the EGFR mutation is G724S. In some embodiments, the EGFR mutation is G724S Ex19del. In some embodiments, the EGFR mutation is G724S L858R. In some embodiments, the EGFR mutation is G779F. In some embodiments, the EGFR mutation is I740dupIPVAK. In some embodiments, the EGFR mutation is K757M L858R. In some embodiments, the EGFR mutation is K757R. In some embodiments, the EGFR mutation is L718Q. In some embodiments, the EGFR mutation is Ex19del. In some embodiments, the EGFR mutation is L718Q L858R. In some embodiments, the EGFR mutation is L718V. In some embodiments, the EGFR mutation is L718V L858R. In some embodiments, the EGFR mutation is L747_S752del A755D. In some embodiments, the EGFR mutation is L747P. In some embodiments, the EGFR mutation is L747S. In some embodiments, the EGFR mutation is L747S L858R. In some embodiments, the EGFR mutation is L747S V774M. In some embodiments, the EGFR mutation is L858R C797S. In some embodiments, the EGFR mutation is L858R L792H. In some embodiments, the EGFR mutation is L858R T854S. In some embodiments, the EGFR mutation is N771G. In some embodiments, the EGFR mutation is R776C. In some embodiments, the EGFR mutation is R776H. In some embodiments, the EGFR mutation is E709_T710del insD S22R. In some embodiments, the EGFR mutation is S752_I759del V769M. In some embodiments, the EGFR mutation is S768I. In some embodiments, the EGFR mutation is S768I L858R. In some embodiments, the EGFR mutation is S768I L861Q. In some embodiments, the EGFR mutation is S768I V769L. In some embodiments, the EGFR mutation is S768I V774M. In some embodiments, the EGFR mutation is T751_I759 delinsN. In some embodiments, the EGFR mutation is V769L. In some embodiments, the EGFR mutation is V769M. In some embodiments, the EGFR mutation is V774M. In some embodiments, the subject was previously treated with a cancer therapy. In some embodiments, the cancer therapy comprised erlotinib, gefitinib, AZD3759, or sapatinib. In some embodiments, the cancer therapy comprised osimertinib, nazartinib, olmutinib, rocelitinib, naquotinib, lazertinib. In some embodiments, the cancer therapy comprised chemotherapy. In some embodiments, the subject was determined to be resistant to the cancer therapy.

In some embodiments of the methods disclosed herein, the method further comprises administering to the subject an additional cancer therapy. In some embodiments, the additional cancer therapy comprises chemotherapy, radiotherapy, or immunotherapy. In some embodiments, the additional cancer therapy comprises an anaplastic lymphoma kinase (ALK) inhibitor. In some embodiments, the ALK inhibitor is brigatinib or AZD3463. In some embodiments, the additional cancer therapy comprises a protein kinase C (PKC) inhibitor. In some embodiments, the PKC inhibitor is ruboxistaurin, midostaurin, or sotrastaurin.

Also disclosed herein, in some aspects, is a method for treating a subject for non-small cell lung cancer, the method comprising administering to a subject an effective amount of a composition comprising (a) a third-generation EGFR inhibitor, (b) a second-generation EGFR inhibitor, (c) a first-generation EGFR inhibitor, or (d) an EGFR inhibitor specific to mutations associated with EGFR exon 20, wherein the subject was previously determined, from analysis of tumor DNA from the subject, to have a classical-like EGFR mutation.

Further disclosed, in some aspects, is a method for treating a subject for non-small cell lung cancer, the method comprising administering to a subject an effective amount of a composition comprising (a) a third-generation EGFR inhibitor, (b) a PKC inhibitor, or (c) an ALK inhibitor, wherein the subject was previously determined, from analysis of tumor DNA from the subject, to have a T790M-like-3S EGFR mutation. In some embodiments, the method does not comprise administering a second-generation EGFR inhibitor to the subject. In some embodiments, the method does not comprise administering a first-generation EGFR inhibitor to the subject.

Disclosed herein, in some aspects, is a method for treating a subject for non-small cell lung cancer, the method comprising administering to a subject an effective amount of a composition comprising (a) a PKC inhibitor or (b) an ALK inhibitor, wherein the subject was previously determined, from analysis of tumor DNA from the subject, to have a T790M-like-3R EGFR mutation. In some aspects, the method further comprises administering a third-generation EGFR inhibitor to the subject. In some aspects, the method does not comprise administering a third-generation EGFR inhibitor to the subject. In some embodiments, the method does not comprise administering a second-generation EGFR inhibitor to the subject. In some embodiments, the method does not comprise administering a first-generation EGFR inhibitor to the subject.

Also disclosed herein, in some aspects, is a method for treating a subject for non-small cell lung cancer, the method comprising administering to a subject an effective amount of a composition comprising (a) a second-generation EGFR inhibitor, or (b) an EGFR inhibitor specific to mutations associated with EGFR exon 20, wherein the subject was previously determined, from analysis of tumor DNA from the subject, to have an exon 20 near-loop insertion EGFR mutation. In some aspects, the method does not comprise administering a first-generation EGFR inhibitor to the subject. In some embodiments, the method does not comprise administering a third-generation EGFR inhibitor to the subject.

Further disclosed, in some embodiments, is a method for treating a subject for non-small cell lung cancer, the method comprising administering to a subject an effective amount of a composition comprising (a) an EGFR inhibitor specific to mutations associated with EGFR exon 20, wherein the subject was previously determined, from analysis of tumor DNA from the subject, to have an exon 20 far-loop insertion EGFR mutation. In some embodiments, the method does not comprise administering a first-generation EGFR inhibitor to the subject. In some embodiments, the method does not comprise administering a second-generation EGFR inhibitor to the subject. In some embodiments, the method does not comprise administering a third-generation EGFR inhibitor to the subject.

Disclosed herein, in some aspects, is a method for treating a subject for non-small cell lung cancer, the method comprising administering to a subject an effective amount of a composition comprising (a) a second-generation EGFR inhibitor, (b) a first-generation EGFR inhibitor, or (c) an EGFR inhibitor specific to mutations associated with EGFR exon 20, wherein the subject was previously determined, from analysis of tumor DNA from the subject, to have a P-loop αC-helix compressing EGFR mutation. In some embodiments, the composition comprises a second-generation EGFR inhibitor. In some embodiments, the method does not comprise administering a third-generation EGFR inhibitor to the subject.

“Individual, “subject,” and “patient” are used interchangeably and can refer to a human or non-human.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The results of treatment can be determined by methods known in the art, such as determination of reduction of pain as measured by reduction of requirement for administration of opiates or other pain medication, determination of reduction of tumor burden, determination of restoration of function, or other methods known in the art.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristics of the disclosure.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1F. EGFR mutations can be separated into four distinct subgroups based on drug sensitivity and structural changes. FIG. 1A. Heat map with unsupervised hierarchical clustering of log (Mutant/WT) ratios from Ba/F3 cells expressing indicated mutations after 72 hours of indicated drug treatment. To determine the mutant/WT ratio, IC₅₀ values for each drug and cell line were calculated and then compared to the average IC₅₀ values of Ba/F3 cells expressing WT EGFR (+10 ng/ml EGF to maintain viability). Squares are representative of the average of n=3 replicates. For co-occurring mutations, the order of exons 1, 2, and 3 were assigned arbitrarily. Groups were assigned based on structural predictions. FIGS. 1B-1E. In silico mutational mapping of (FIG. 1B) classical-like, (FIG. 1C) T790M-like, (FIG. 1D) exon 20 loop insertion (red/blue) and WT (grey/green) and (FIG. 1E) PACC mutants. FIG. 1F. Dot plot of Rho values from Spearman correlations of mutations vs exon based group averages or structure-function based averages for each drug. Dots are representative of each mutation, bars are representative of the average Rho value±standard deviation (SD) and p-value was determined using a paired students' t-test.

FIG. 2. Structure-function based groupings are more predictive of drug and mutation sensitivity compared to exon based groupings. Bar plot of Spearman rho values for indicated mutations compared to exon based groups (yellow) or structure-function based groups (green). The delta of the two rho values is shown as an overlapped grey bar. When the delta bar shifts to the right, the spearman rho value was higher for structure-function based groups, and when the grey bar shifts to the left, the spearman rho value was higher for the exon based groups.

FIGS. 3A-3B. Heat maps generated through supervised clustering by structure-function based groups cluster drug sensitivity better than exon based groups. FIGS. 3A-3B. Heat maps supervised clustering by (FIG. 3A) exon based or (FIG. 3B) structure-function based groups of log (Mutant/WT) ratios from Ba/F3 cells expressing indicated mutations after 72 hours of indicated drug treatment. To determine the mutant/WT ratio, IC₅₀ values for each drug and cell line were calculated and then compared to the average IC₅₀ values of Ba/F3 cells expressing WT EGFR (+10 ng/ml EGF to maintain viability). Squares are representative of the average of n=3 replicates. For co-occurring mutations, the order of exons 1, 2, and 3 were assigned arbitrarily. Groups were assigned based on structural predictions.

FIGS. 4A-4G. Classical-like EGFR mutations are not predicted to alter the drug-binding pocket and are most sensitive to third-generation EGFR TKIs. FIGS. 4A-4B. In silico models of WT EGFR (PDB 2ITX) visualized as both a (FIG. 4A) a ribbon and (FIG. 4B) space filling models. Residues important in receptor signaling and drug binding are highlighted.

FIGS. 4C-4D. Overlapped in silico models of (FIG. 4C) WT (grey) and L861R (blue) and (FIG. 4D) space filing model of L861Q demonstrate the R861 substitution is distal from the drug binding pocket and has minimal impact on the overall structure of EGFR compared to WT. FIG. 4E. Dot plot of mutant/WT IC₅₀ values of Ba/F3 cells expressing classical-like EGFR mutations and treated with indicated classes of EGFR TKIs. Dots are representative of average of n=3 replicate mutant/WT IC₅₀ values of individual cell lines expressing classical-like mutations with individual drugs. Bars are representative of average mutant/WT IC₅₀ values±SEM for each class of EGFR TKI and all classical-like cell lines. p-values were determined by ANOVA analysis with unequal SD as determined by Brown-Forsythe test to determined differences in SD. Holm-Sidak's multiple comparisons test was used to determine differences between groups. FIG. 4F. Tumor growth curves for PDXs harboring EGFR L858R E709K complex mutation treated with indicated inhibitors. Tumors were measured three times per week and symbols are average of tumor volumes±SEM. Mice were randomized into six groups. Mice received drug 5 days per week, and mice were euthanized at day 28 to harvest tumors. FIG. 4G. Dot plot of percent change in tumor volume on day 28 of tumors described in FIG. 4F. Dots are representative of each tumor, and bars are representative of average±SEM for each group. Statistical differences were determined by ordinary one-way ANOVA with post-hoc Tukey's multiple comparisons test to determined differences between groups.

FIGS. 5A-5C. Exon 20 loop insertions are a distinct class of EGFR mutations. FIG. 5A. Dot plot of mutant/WT IC₅₀ values of Ba/F3 cells expressing exon 20 loop insertion mutations and treated with indicated classes of EGFR TKIs. Dots are representative of average of n=3 replicate mutant/WT IC₅₀ values of individual cell lines expressing exon 20 loop insertion mutations with individual drugs. Bars are representative of average mutant/WT IC₅₀ values±SEM for each class of EGFR TKI and all exon 20 loop insertion cell lines. p-values were determined by ANOVA analysis with unequal SD as determined by Brown-Forsythe test to determined differences in SD. Holm-Sidak's multiple comparisons test was used to determine differences between groups. FIG. 5B. Tumor growth curves for PDXs harboring EGFR S768dupSVD exon 20 loop insertion mutation treated with indicated inhibitors. Tumors were measured three times per week and symbols are average of tumor volumes±SEM. Mice were randomized into four groups. Mice received drug 5 days per week, and mice were euthanized at day 21 to harvest tumors. FIG. 5C. Dot plot of percent change in tumor volume on day 21 of tumors described in FIG. 5B. Dots are representative of each tumor, and bars are representative of average±SEM for each group. Statistical differences were determined by ordinary one-way ANOVA with post-hoc Tukey's multiple comparisons test to determined differences between groups.

FIGS. 6A-6C. Drug repurposing can overcome T790M-like resistance mutations. FIG. 6A. Heat map with unsupervised hierarchical clustering of log (Mutant/WT) ratios from Ba/F3 cells expressing indicated mutations after 72 hours of indicated drug treatment. Squares are representative of the average of n=3 replicates. For co-occurring mutations, the order of exons 1, 2, and 3 were assigned arbitrarily. Groups were assigned based on hierarchal clustering and known resistance mutations. FIGS. 6B-6C. Dot plot of mutant/WT IC₅₀ values of Ba/F3 cells expressing (FIG. 6B) T790M-like-3S (third-generation EGFR TKI sensitive) and (FIG. 6C) T790M-like-3R (third-generation EGFR TKI resistant) EGFR mutations and treated with indicated classes of EGFR TKIs. Dots are representative of average of n=3 replicate mutant/WT IC₅₀ values of individual cell lines expressing classical-like mutations with individual drugs. Bars are representative of average mutant/WT IC₅₀ values±SEM for each class of EGFR TKI and all cell lines. p-values were determined by ANOVA analysis with unequal SD as determined by Brown-Forsythe test to determined differences in SD. Holm-Sidak's multiple comparisons test was used to determine differences between groups.

FIGS. 7A-7G. PACC mutations are robustly sensitive to second-generation TKIs. FIG. 7A. In silico modeling of EGFR G179S (PDB 2ITN, purple) with osimertinib in the reactive conformation (green) and predicted conformation with G719S (orange) demonstrate destabilization of TKI-protein interactions at the indole ring. FIG. 7B. Dot plot of mutant/WT IC₅₀ values of Ba/F3 cells expressing PACC mutations and treated with indicated classes of EGFR TKIs. Dots are representative of average of n=3 replicate mutant/WT IC₅₀ values of individual cell lines expressing PACC mutations with individual drugs. Bars are representative of average mutant/WT IC₅₀ values±SEM for each class of EGFR TKI and all PACC cell lines. p-values were determined by ANOVA analysis with unequal SD as determined by Brown-Forsythe test to determined differences in SD. Holm-Sidak's multiple comparisons test was used to determine differences between groups. FIG. 7C. Tumor growth curves for PDXs harboring EGFR S768dupSVD exon 20 loop insertion mutation treated with indicated inhibitors. Tumors were measured three times per week and symbols are average of tumor volumes±SEM. Mice were randomized into six groups. Mice received drug 5 days per week, and mice were euthanized at day 28 to harvest tumors. FIG. 7D. Computed tomography (CT) scans of a patient with NSCLC harboring a G719S E709K complex mutation before aftatinib treatment and four weeks after afatinib treatment. Arrows indicate resolved pleural effusion in the right lobe and reduced pleural effusion and tumor size in the left lobe. FIG. 7E. Heat map with unsupervised hierarchical clustering of log (Mutant/WT) ratios from Ba/F3 cells expressing indicated mutations after 72 hours of indicated drug treatment. Squares are representative of the average of n=3 replicates. For co-occurring mutations, the order of exons 1, 2, and 3 were assigned arbitrarily. Groups were assigned based on predicted mutational impact. FIG. 7F. Dot plot of mutant/WT IC₅₀ values of Ba/F3 cells expressing classical EGFR mutations (white bars) or classical EGFR mutations and acquired PACC mutations (colored bars) treated with indicated classes of EGFR TKIs. Dots are representative of average of n=3 replicate mutant/WT IC₅₀ values of individual cell lines expressing indicated mutations with individual drugs. Bars are representative of average mutant/WT IC₅₀ values±SEM for each class of EGFR TKI and indicated cell lines. p-values were determined by ANOVA analysis with unequal SD as determined by Brown-Forsythe test to determined differences in SD. Holm-Sidak's multiple comparisons test was used to determine differences between groups. FIG. 7G. In silico modeling of EGFR Ex19del G796S, purple) with osimertinib in the reactive conformation (blue) and predicted conformation with G719S (orange) demonstrate destabilization of TKI-protein interactions in the hinge region (yellow), displacing the reactive group of osimertinib (arrow).

FIGS. 8A-8F. PACC mutations alter the orientation of the P-loop and/or α-C-helix and are sensitive to second-generation TKIs. FIG. 8A. Overlap of G719S (PDB 2ITN, green) and WT EGFR (PDB 2ITX, grey) crystal structures demonstrate a significant shift of F723 (red arrow) in the P-loop orienting the benzyl ring in a downward position condensing the P-loop in the drug binding pocket. Further, G719S has an inward shift of the α-C-helix compared to the WT crystal structure. FIG. 8B. Space filling model of G719S (PDB 2ITN) with P-loop (red), α-C-helix (blue), hinge region (orange), C797 (yellow), and DFG motif (green) highlighted to demonstrate steric hindrance of drug binding pocket caused by shifted P-loop. FIG. 8C. In silico homology model of EGFR L718Q (pink) with predicted osimertinib structure demonstrates that Q718 hinders the interaction of osimertinib (green) with M793 and shifts the Michael acceptor (reactive group, green arrow) out of alignment with C797 (yellow arrow). FIG. 8D. In silico modeling of EGFR G719S. FIG. 8E. Dot plot of percent change in tumor volume on day 28 of tumors described in FIG. 3C. Dots are representative of each tumor, and bars are representative of average±SEM for each group. Statistical differences were determined by ordinary one-way ANOVA with post-hoc Tukey's multiple comparisons test to determined differences between groups. FIG. 8F. In silico modeling of EGFR Ex19del G796S (purple).

FIGS. 9A-9D. Structure-function groups better predict patient outcomes than exon based groups. FIG. 9A. Kaplan-Meier plot of duration of afatinib treatment of patients with NSCLC tumors harboring atypical EGFR mutations (N=358 patients) stratified by structure based groups. FIG. 9B. Forest plot of hazard ratios calculated from Kaplan-Meier plots in FIG. 9A. Hazard ratios and p-value were calculated using the Mantel-Cox, Log-Rank method. Data are representative of the Hazard Ratio±95% CI. FIGS. 9A-9B. Classical-like N=58, T790M-like N=68, Ex20ins N=76, and PACC N=156. When mutations were not explicitly stated, those patients were excluded from the structure-function based analysis. FIG. 9C. Kaplan-Meier plot of PFS of patients with NSCLC tumors harboring PACC mutations (N=44 treated with first- (N=13 patients), second- (N=21 patients), or third-generation (N=10 patients) EGFR TKIs.

FIG. 9D. Forest plot of hazard ratios calculated from Kaplan-Meier plots in panel C and Extended Data Fig. E. Hazard ratios and p-value were calculated using the Mantel-Cox, Log-Rank method. PACC N=44: 1^{st} N=13, 2^{nd} N=21, and 3^{rd} N=10, non-PACC N=40, 1^{st} N=20, 2^{nd} N=6, and 3^{rd} N=14.

FIGS. 10A-10E. Structure-function groups identify patients with greater benefit to second-generation TKIs than exon based groups. FIGS. 10A-10B. Overall response rate to afatinib stratified by (FIG. 10A) structure-function based groups (N=507: Classical-like N=91, T790M-like N=103, Ex20ins N=120, and PACC N=193) or (FIG. 10B) exon based groups (N=528: Exon 18 N=133, Exon 19 N=22, Exon 20 N=294, Exon 21 N=79). When mutations were not explicitly stated (N=21), those patients were excluded from the structure-function based analysis. FIG. 10C. Kaplan-Meier plot of duration of afatinib treatment of patients with NSCLC tumors harboring atypical EGFR mutations (N=364 patients) stratified by exon based groups. FIG. 10D. Forest plot of hazard ratios calculated from Kaplan-Meier plots in FIG. 10C. Hazard ratios and p-value were calculated using the Mantel-Cox, Log-Rank method. Data are representative of the Hazard Ratio±95% CI. FIGS. 10C-10D. Exon 18 N=87, Exon 19 N=19, Exon 20 N=195, and Exon 21 N=63). FIG. 10E. Kaplan-Meier plot of PFS of patients with NSCLC tumors harboring non-PACC atypical EGFR mutations (N=40) treated with first- (N=20), second- (N=6), or third-generation (N=14) EGFR TKIs.

FIG. 11 shows representative space-filling models of the disclosed EGFR mutation subgroups showing changes in overall shape of the drug-binding pocket. The most common mutations are shown for each group, and drug sensitivity or selectivity is listed from most selective or sensitive to resistant.

### DETAILED DESCRIPTION

The present disclosure is based, at least in part, on the surprising discovery that four distinct structure-function based groups of EGFR mutations are more predictive of patient outcomes after treatment with a cancer therapy, for example, a kinase inhibitor, than are classical groupings of mutations by the exon in the EGFR gene in which the mutations appear. Notably, each of the four groups of mutations correspond to distinct classes of drugs that are specifically effective for treating cancers expressing EGFR mutations from each of the groups that could be repurposed for the treatment of patients. Further, the structure-function based groups are better at predicting mutations with similar sensitivities to drug classes than exon-based groupings.

Accordingly, in some embodiments, disclosed are methods for treating a subject for cancer, e.g., lung cancer, the method comprising administering an effective amount of one or more kinase inhibitors from one or more kinase classes to a subject determined, from analysis of tumor DNA from the subject, to have an EGFR mutation, wherein the EGFR mutation is a classical-like mutation, an exon 20 loop insertion-specific mutation, a T790M-like-sensitive (T790M-like-3S) mutation, a T790M-like-resistant (T790M-like-3R) mutation, or a P-loop and αC-helix compressing mutation. Also disclosed are methods for treating a subject for cancer, e.g., lung cancer, the method comprising: (a) detecting an EGFR mutation in tumor DNA from the subject, wherein the EGFR mutation is a classical-like mutation, an exon 20 loop insertion-specific mutation (e.g., Exon20ins-NL or Exon20ins-FL), a T790M-like-sensitive (T790M-like-3S) mutation, a T790M-like-resistant (T790M-like-3R) mutation, or a P-loop and α-C-helix compressing mutation; and (b) administering an effective amount of one or more kinase inhibitors from one or more kinase classes depending on the detected EGFR mutations.

### I. Therapeutic Methods

Aspects of the disclosure are directed to compositions comprising therapeutically effective amounts of one or more cancer therapies and administration of such compositions to a subject or patient in need thereof. In some embodiments, the one or more cancer therapies comprise one or more kinase inhibitors.

The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration. The cancer therapies may be administered via the same or different routes of administration.

The term “cancer,” as used herein, may be used to describe a solid tumor, metastatic cancer, or non-metastatic cancer. In certain embodiments, the cancer may originate in the blood, bladder, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, disclosed are methods for treating lung cancer. In some embodiments, the lung cancer is non-small cell lung cancer. In some embodiments, the non-small cell lung cancer is adenocarcinoma. In some embodiments, the non-small cell lung cancer is squamous cell carcinoma. In some embodiments, the non-small cell lung cancer is large cell carcinoma. In some embodiments, the non-small cell lung cancer is adenosquamous carcinoma. In some embodiments, the non-small cell lung cancer is sarcomatoid carcinoma. In some embodiments, the lung cancer is small cell lung cancer. In some aspects, disclosed are methods for treating cancer that is not lung cancer.

In some embodiments, the cancer therapy comprises a local cancer therapy. In some embodiments, the cancer therapy comprises a systemic cancer therapy. In some embodiments, the cancer therapy excludes a systemic cancer therapy. In some embodiments, the cancer therapy excludes a local cancer therapy.

### A. Kinase Inhibitors

In some embodiments, the one or more cancer therapies comprise one or more kinase inhibitors. In some embodiments, the disclosed methods comprise administration of one or more kinase inhibitors to a subject or patient in need thereof. As used herein, kinase inhibitors describe pharmaceutical compounds that inhibit kinases. Examples of kinases which may be inhibited by kinase inhibitors of the disclosure include epidermal growth factor receptor (EGFR), anaplastic lymphoma kinase (ALK), and protein kinase C (PKC). Kinases are a part of many cell functions, including cell signaling, growth, and division. Specifically, tyrosine kinases are responsible for the activation of many proteins by signal transduction cascades resulting from phosphorylation of the proteins by tyrosine kinases. Kinase inhibitors inhibit the phosphorylation and subsequent activation of proteins by tyrosine kinases.

Kinase inhibitors operate by competing with adenosine triphosphate, the phosphorylating entity, the substrate, or both, or acting in an allosteric fashion, namely binding to a site outside the active site, affecting its activity by a conformational change. Recently, TKIs have been shown to deprive tyrosine kinases of access to the Cdc37-Hsp90 molecular chaperone system on which they depend for their cellular stability, leading to their ubiquitylation and degradation.

The amount of the one or more kinase inhibitors delivered to the patient may be variable. In one suitable embodiment, the kinase inhibitors may be administered in an amount effective to cause arrest or regression of the cancer in a host. In other embodiments, the kinase inhibitors may be administered in an amount that is anywhere between 2 to 10,000 fold less than the chemotherapeutic effective dose of the kinase inhibitors. For example, the kinase inhibitors may be administered in an amount that is about 20 fold less, about 500 fold less or even about 5000 fold less than the chemotherapeutic effective dose of the kinase inhibitors.

The kinase inhibitors of the disclosure can be tested in vivo for the desired therapeutic activity alone or in combination with another cancer therapy, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

The compositions of the one or more kinase inhibitor compositions may or may not be tailored to address any kinase inhibitor sensitivity or resistance of a cancer as determined based on analysis of the genome of a subject having cancer for one or more mutations in the EGFR gene of the subject. The compositions may be given to a subject without having prior analysis of their genome. The kinase inhibitor compositions may comprise any one or more kinase inhibitors associated with an efficacious therapy for treating cancer.

The subject may be given one or more kinase inhibitor compositions, including compositions that comprise one or more kinase inhibitors that overcome any sensitivity or resistance of a cancer as determined based on analysis of the genome of a subject having cancer for one or more mutations in the EGFR gene of the subject. The kinase inhibitors may be given to treat cancer and/or enhance therapy to treat cancer.

The kinase inhibitor composition can be administered alone or in combination with one or more additional therapeutic agents disclosed herein. Administration “in combination with” one or more additional therapeutic agents includes both simultaneous (at the same time) and consecutive administration in any order. The kinase inhibitor composition and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

In some embodiments, the kinase inhibitor of the present disclosure is a tyrosine kinase inhibitor (TKI). Embodiments of the disclosure comprise administration of at least 1, 2, 3, 4, 5, or 6 classes of TKIs to a subject having cancer. In some embodiments, the 1, 2, 3, 4, 5, or 6 TKI classes comprise first-generation EGFR TKIs, second-generation EGFR TKIs, third-generation EGFR TKIs, EGFR TKIs specific to mutations associated with EGFR exon 20, anaplastic lymphoma kinase (ALK) inhibitors, or protein kinase C (PKC) inhibitors.

In some embodiments, the TKIs are first-generation EGFR TKIs, which include but are not limited to Erlotinib, Geftinib, AZD3759, Sapatinib, Lapatinib, Tucatinib, and icotinib. In some aspects, the first-generation EGFR inhibitor is Erlotinib. In some aspects, the first-generation EGFR inhibitor is Geftinib. In some aspects, the first-generation EGFR inhibitor is AZD3759. In some aspects, the first-generation EGFR inhibitor is Sapatinib. In some aspects, the first-generation EGFR inhibitor is Lapatinib. In some aspects, the first-generation EGFR inhibitor is Tucatinib. In some aspects, the first-generation EGFR inhibitor is icotinib. Any one or more of these EGFR inhibitors may be excluded from certain embodiments. As used herein, a “first-generation EGFR TKI” (also “first generation EGFR TKI,” “first-generation EGFR inhibitor,” and “first generation EGFR inhibitor,” used interchangeably herein) describes an EGFR inhibitor that is capable of non-covalently binding to an EGFR protein and is not capable of covalently binding to EGFR. In some embodiments, the TKIs are second-generation EGFR TKIs, which include but are not limited to Afatinib, Dacomitinib, Neratinib, Tarlox-TKI, Tarloxotinib, BDTX189, and sutetinib. In some aspects, the second-generation EGFR inhibitor is Afatinib. In some aspects, the second-generation EGFR inhibitor is Dacomitinib. In some aspects, the second-generation EGFR inhibitor is Neratinib. In some aspects, the second-generation EGFR inhibitor is Tarlox-TKI. In some aspects, the second-generation EGFR inhibitor is Tarloxotinib. In some aspects, the second-generation EGFR inhibitor is BDTX189. In some aspects, the second-generation EGFR inhibitor is sutetinib. Any one or more of these EGFR inhibitors may be excluded from certain embodiments. As used herein, a “second-generation EGFR TKI” (also “second generation EGFR TKI,” “second-generation EGFR inhibitor,” or “second generation EGFR inhibitor,” used interchangeably herein) describes an EGFR inhibitor capable of covalently binding to a wild type EGFR protein and certain mutant EGFR proteins but incapable of binding to (or having reduced affinity for) a T790M mutant EGFR. In some embodiments, the TKIs are third-generation EGFR TKIs, which include but are not limited to Osimertinib, Nazartinib, Olmutinib, Rocelitinib, Naquotinib, Lazertinib, WZ4002, almonertinib, furmonertinib, abivertinib, alflutinib, mavelertinib, abivertinib, olafertinib, and rezivertinib. In some aspects, the third-generation EGFR inhibitor is Osimertinib. In some aspects, the third-generation EGFR inhibitor is Nazartinib. In some aspects, the third-generation EGFR inhibitor is Olmutinib. In some aspects, the third-generation EGFR inhibitor is Rocelitinib. In some aspects, the third-generation EGFR inhibitor is Naquotinib. In some aspects, the third-generation EGFR inhibitor is Lazertinib. In some aspects, the third-generation EGFR inhibitor is WZ4002. In some aspects, the third-generation EGFR inhibitor is almonertinib. In some aspects, the third-generation EGFR inhibitor is furmonertinib. In some aspects, the third-generation EGFR inhibitor is abivertinib. In some aspects, the third-generation EGFR inhibitor is alflutinib. In some aspects, the third-generation EGFR inhibitor is mavelertinib. In some aspects, the third-generation EGFR inhibitor is abivertinib. In some aspects, the third-generation EGFR inhibitor is olafertinib. In some aspects, the third-generation EGFR inhibitor is rezivertinib. Any one or more of these EGFR inhibitors may be excluded from certain embodiments. As used herein, a “third-generation EGFR TKI” (also “third generation EGFR TKI,” “third-generation EGFR inhibitor,” and “third generation EGFR inhibitor,” used interchangeably herein) describes an EGFR inhibitor capable of covalently binding to a T790M mutant EGFR protein, in addition to wild type EGFR and other mutant EGFR proteins. In some embodiments, the EGFR TKIs are EGFR TKIs specific to mutations associated with EGFR exon 20, which include but are not limited to TAS 6417, AZ5104, TAK-788 (mobocertinib), and DZD9008. In some aspects, the EGFR inhibitor specific to a mutation associated with EGFR exon 20 is TAS 6417. In some aspects, the EGFR inhibitor specific to a mutation associated with EGFR exon 20 is AZ5104. In some aspects, the EGFR inhibitor specific to a mutation associated with EGFR exon 20 is TAK-788 (mobocertinib). In some aspects, the EGFR inhibitor specific to a mutation associated with EGFR exon 20 is DZD9008. Any one or more of these EGFR inhibitors may be excluded from certain embodiments. An EGFR TKI (or “EGFR inhibitor”) specific to mutations associated with EGFR exon 20 describes an EGFR inhibitor capable of covalently binding to an Exon 20-insertion mutant EGFR. In some aspects, a TKI of the disclosure is not an EGFR inhibitor. In some embodiments, the TKIs are ALK inhibitors, which include but are not limited to AZD3463, Brigatinib, Crizotinib, Ceritinib, Alectinib, Lorlatinib, Ensartinib, Entrectinib, Repotrectinib, Belizatinib, Alkotinib, Foritinib, CEP-37440, TQ-B3139, PLB1003, TPX-0131, and ASP-3026. In some aspects, the ALK inhibitor is AZD3463. In some aspects, the ALK inhibitor is Brigatinib. In some aspects, the ALK inhibitor is Crizotinib. In some aspects, the ALK inhibitor is Ceritinib. In some aspects, the ALK inhibitor is Alectinib. In some aspects, the ALK inhibitor is Lorlatinib. In some aspects, the ALK inhibitor is Ensartinib. In some aspects, the ALK inhibitor is Entrectinib. In some aspects, the ALK inhibitor is Repotrectinib. In some aspects, the ALK inhibitor is Belizatinib. In some aspects, the ALK inhibitor is Alkotinib. In some aspects, the ALK inhibitor is Foritinib. In some aspects, the ALK inhibitor is CEP-37440. In some aspects, the ALK inhibitor is TQ-B3139. In some aspects, the ALK inhibitor is PLB1003. In some aspects, the ALK inhibitor is TPX-0131. In some aspects, the ALK inhibitor is ASP-3026. Any one or more of these ALK inhibitors may be excluded from certain embodiments. In some embodiments, the TKIs are PKC inhibitors, which include but are not limited to Ruboxistaurin, Midostaurin, Sotrastaurin, Chelerythrine, Miyabenol C, Myricitrin, Gossypol, Verbascoside, BIM-1, Bryostatin 1, and Tamoxifen. In some aspects, the PKC inhibitor is Ruboxistaurin, Midostaurin, Sotrastaurin, Chelerythrine, Miyabenol C, Myricitrin, Gossypol, Verbascoside, BIM-1, Bryostatin 1, Tamoxifen. Any one or more of these PKC inhibitors may be excluded from certain embodiments.

In some embodiments, the TKI is Erlotinib, Geftinib, AZD3759, Sapatinib, Lapatinib, Tucatinib, Afatinib, Dacomitinib, Neratinib, Tarlox-TKI, TAS 6417, AZ5104, TAK-788 (mobocertinib), Osimertinib, Nazartinib, Olmutinib, Rocelitinib, Naquotinib, Lazertinib, AZD3463, Brigatinib, Ruboxistaurin, Midostaurin, Sotrastaurin, or a combination thereof. Embodiments of the disclosure comprise administration of at least 1, 2, 3, 4, 5, or more TKIs to a subject having cancer. In some embodiments, the one or more TKIs comprise two or more of Erlotinib, Geftinib, AZD3759, Sapatinib, Afatinib, Dacomitinib, Neratinib, Tarlox-TKI, TAS 6417, AZ5104, TAK-788 (mobocertinib), Osimertinib, Nazartinib, Olmutinib, Rocelitinib, Naquotinib, Lazertinib, AZD3463, Brigatinib, Ruboxistaurin, Midostaurin, and Sotrastaurin.

In some embodiments, the EGFR inhibitor is Geftinib, AZD3759, Sapatinib, Lapatinib, Tucatinib, Afatinib, Dacomitinib, Neratinib, Tarlox-TKI, TAS 6417, AZ5104, TAK-788 (mobocertinib), Osimertinib, Nazartinib, Olmutinib, Rocelitinib, Naquotinib, or Lazertinib. Embodiments of the disclosure comprise administration of at least 1, 2, 3, 4, 5, or more EGFR inhibitors to a subject having cancer.

In some embodiments, the methods disclosed herein further comprise administering to the subject an additional cancer therapy. In some embodiments, the additional cancer therapy comprises chemotherapy, radiotherapy, or immunotherapy. In some embodiments, the additional cancer therapy comprises an ALK inhibitor. In some embodiments, the ALK inhibitor is brigatinib or AZD3463. In some embodiments, the additional cancer therapy comprises a PKC inhibitor. In some embodiments, the PKC inhibitor is ruboxistaurin, midostaurin, or sotrastaurin.

In some embodiments, a kinase inhibitor of the disclosure is an EGFR inhibitor designed to be active in one or more types of EGFR-mutant cancers. In some embodiments, a kinase inhibitor is an EGFR inhibitor designed to be active in classical-like EGFR mutant lung cancer. In some embodiments, a kinase inhibitor is an EGFR inhibitor designed to be active in T790M-like-3S EGFR mutant lung cancer. In some embodiments, a kinase inhibitor is an EGFR inhibitor designed to be active in T790M-like-3R EGFR mutant lung cancer. In some embodiments, a kinase inhibitor is an EGFR inhibitor designed to be active in Exon20ins-NL EGFR mutant lung cancer. In some embodiments, a kinase inhibitor is an EGFR inhibitor designed to be active in Exon20ins-FL EGFR mutant lung cancer. In some embodiments, a kinase inhibitor is an EGFR inhibitor designed to be active in P-loop αC-helix compressing EGFR mutant lung cancer.

In some embodiments, a kinase inhibitor of the disclosure is an EGFR inhibitor that is preferentially effective in one or more types of EGFR-mutant cancers. An EGFR inhibitor that is “preferentially effective” in a type of EGFR-mutant cancers describes an EGFR inhibitor that has an increased efficacy in killing cancer cells having an EGFR mutation of the given type compared with cancer cells having an EGFR mutation of a different type. For example, an EGFR inhibitor that is preferentially effective in classical-like EGFR mutant lung cancer describes an EGFR inhibitor that has an increased efficacy in killing cancer cells having a classical-like EGFR mutation (e.g., A702T) compared with cancer cells having a P-loop aC-helix compressing EGFR mutation (e.g., V769L). In some embodiments, a kinase inhibitor is an EGFR inhibitor that is preferentially effective in classical-like EGFR mutant lung cancer. In some embodiments, a kinase inhibitor is an EGFR inhibitor that is preferentially effective in T790M-like-3S EGFR mutant lung cancer. In some embodiments, a kinase inhibitor is an EGFR inhibitor that is preferentially effective in T790M-like-3R EGFR mutant lung cancer. In some embodiments, a kinase inhibitor is an EGFR inhibitor that is preferentially effective in Exon20ins-NL EGFR mutant lung cancer. In some embodiments, a kinase inhibitor is an EGFR inhibitor that is preferentially effective in Exon20ins-FL EGFR mutant lung cancer. In some embodiments, a kinase inhibitor is an EGFR inhibitor that is preferentially effective in P-loop αC-helix compressing EGFR mutant lung cancer.

### B. Radiotherapy

In some embodiments, a radiotherapy, such as ionizing radiation, is administered to a subject as a therapeutic agent. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of radiotherapy administered to a subject may be presented as a total dose of radiotherapy, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### C. Cancer Immunotherapy

In some embodiments, the methods comprise administration of a cancer immunotherapy as a therapeutic agent. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### 2. Activation of Co-Stimulatory Molecules

In some embodiments, the immunotherapy comprises an agonist of a co-stimulatory molecule. In some embodiments, the agonist comprises an activator of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonist include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain and an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### Example CAR-T Therapies Include Tisagenlecleucel (Kymriah) and Axicabtagene Ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### D. Oncolytic Virus

In some embodiments, the additional therapy comprises an oncolytic virus as a therapeutic agent. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### E. Polysaccharides

In some embodiments, the additional therapy comprises polysaccharides as a therapeutic agent. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### F. Neoantigens

In some embodiments, the additional therapy comprises neoantigen administration as a therapeutic agent. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### G. Chemotherapies

In some embodiments, the additional therapy comprises a chemotherapy as a therapeutic agent. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### H. Surgery

In some embodiments, the additional therapy comprises surgery as a therapeutic agent. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Anti-EGFR Antibodies and Antibody-Drug Conjugates

In certain aspects, one or more anti-EGFR antibodies or antibody-like molecules (including, for example, antibody-drug conjugates) are contemplated for use in combination with one or more EGFR inhibitors of the disclosure. For example, therapeutic methods of the disclosure may include treatment of a patient with one or more EGFR inhibitors (which inhibitors may be selected based on the patient's EGFR mutation classification) in combination with one or more anti-EGFR antibodies (or antibody-drug conjugates thereof). Anti-EGFR antibodies are known in the art and include, for example, cetuximab and amivantamab. In particular aspects, disclosed is a method for treatment of a subject having an exon 20 loop insertion EGFR mutant cancer (e.g., a cancer having a mutation of Tables 2.1-2.5) comprising providing both a second-generation EGFR inhibitor and amivantamab (or an antibody-drug conjugate thereof).

### J. Anti-Angiogenic Agents

In certain aspects, one or more anti-angiogenic agents are contemplated for use in combination with one or more EGFR inhibitors of the disclosure. For example, therapeutic methods of the disclosure may include treatment of a patient with one or more EGFR inhibitors (which inhibitors may be selected based on the patient's EGFR mutation classification) in combination with one or more anti-angiogenic agents. Anti-angiogenic agents are known in the art and include, for example, ramucirumab and bevacizumab.

### K. Other Agents

It is contemplated that other therapeutic agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### II. Cancer Treatment

Aspects of the present disclosure are directed to methods comprising treatment of a subject suffering from, or suspected of having, cancer. In some embodiments, the cancer is lung cancer. In some embodiments, the lung cancer is non-small cell lung cancer. In some embodiments, the non-small cell lung cancer is adenocarcinoma. In some embodiments, the non-small cell lung cancer is squamous cell carcinoma. In some embodiments, the non-small cell lung cancer is large cell carcinoma. In some embodiments, the non-small cell lung cancer is adenosquamous carcinoma. In some embodiments, the non-small cell lung cancer is sarcomatoid carcinoma. In some embodiments, the lung cancer is small cell lung cancer. In some embodiments, the cancer is not lung cancer.

In particular embodiments, the tumor DNA of a subject having cancer is analyzed or measured or evaluated for one or more mutations in the EGFR gene, irrespective of which mutations are actually present and/or absent. The one or more mutations in the EGFR gene may be analyzed or measured in any suitable manner. For example, a mutation in the EGFR gene (an “EGFR mutation”) may be identified by sequencing DNA and/or RNA (e.g., mRNA) from a sample. In particular cases, a cancer having one or more mutations in the EGFR gene has an increased sensitivity (or decreased resistance) to one or more kinase inhibitors from one or more kinase inhibitor classes. In particular cases, a cancer having one or more mutations in the EGFR gene has a decreased sensitivity (or increased resistance) to one or more kinase inhibitors from one or more kinase inhibitor classes.

In some embodiments, the disclosed methods comprise treating a subject suffering from cancer (e.g., lung cancer such as non-small cell lung cancer) by administering a therapeutically effective amount of a composition comprising one or more kinase inhibitors from one or more kinase inhibitor classes. In some embodiments, the one or more kinase inhibitors are of the same class. In some embodiments, the one or more kinase inhibitors are of different classes. As used herein, the term “therapeutically effective amount” is synonymous with “effective amount,” “therapeutically effective dose,” and/or “effective dose,” and refers to an amount of an agent sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. For instance, in some embodiments, the effective amount refers to the amount of a composition comprising one or more kinase inhibitors that can treat or prevent cancer in a subject. The effective amount may vary depending on the organism or individual treated. The appropriate effective amount to be administered for a particular application of the disclosed methods can be determined by those skilled in the art, using the guidance provided herein. As used herein, the terms “treatment,” “treat,” or “treating” refers to intervention in an attempt to alter the natural course of the subject being treated, and may be performed either for prophylaxis or during the course of pathology of a disease or condition. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, lowering the rate of disease progression, amelioration or palliation of the disease state, and remission or improved prognosis.

Methods of the disclosure include compositions and methods for treating cancer (e.g., lung cancer such as non-small cell lung cancer) with one or more kinase inhibitors from one or more kinase inhibitor classes based on sensitivity (or resistance) of the cancer to the one or more kinase inhibitors. In some embodiments, the cancer is more sensitive (or less resistant) to one or more kinase inhibitors from one or more kinase inhibitor classes than to kinase inhibitors from different kinase inhibitor classes. In some cases, the method is employed for a subject where it is uncertain whether or not the cancer is more sensitive (or less resistant) to one or more kinase inhibitors from one or more kinase inhibitor classes, whereas in other cases the method is employed for a subject where it is known that the cancer is more sensitive (or less resistant) to one or more kinase inhibitors from one or more kinase inhibitor classes. In other cases, it has been determined that the cancer is more sensitive (or less resistant) to one or more kinase inhibitors from one or more kinase inhibitor classes for the subject, but the methods of the disclosure are still employed as a routine matter or in the general therapeutic interest of the subject. In some cases, the method is employed for a subject where it is uncertain whether or not the cancer is less sensitive (or more resistant) to one or more kinase inhibitors from one or more kinase inhibitor classes, whereas in other cases the method is employed for a subject where it is known that the cancer is less sensitive (or more resistant) to one or more kinase inhibitors from one or more kinase inhibitor classes. In other cases, it has been determined that the cancer is less sensitive (or more resistant) to one or more kinase inhibitors from one or more kinase inhibitor classes for the subject, but the methods of the disclosure are still employed as a routine matter or in the general therapeutic interest of the subject.

The selection of one or more kinase inhibitors from one or more kinase inhibitor classes used to treat cancer (e.g., lung cancer) may be as a result of analysis of tumor DNA from a subject having the cancer for one or more mutations in the EGFR gene. In some cases, the selection of one or more kinase inhibitors from one or more kinase inhibitor classes is a result of analysis of tumor DNA of a subject having cancer for one or more mutations in the EGFR gene of the subject, and the outcome of the analysis determines the one or more kinase inhibitors from one or more kinase inhibitor classes used to treat the cancer. In some embodiments, the one or more mutations are classical-like EGFR mutations. In some embodiments, the one or more mutations are exon 20 loop insertion (ex20ins) mutations. In some embodiments, the one or more mutations are T790M-like-3S mutations. In some embodiments, the one or more mutations are T790M-like-3R mutations. In some embodiments, the one or more mutations are P-loop and α-C-helix compressing (PACC) mutations.

### A. Classical-Like EGFR Mutations

In some cases, the subject having cancer (e.g., lung cancer such as non-small cell lung cancer) is determined to have one or more classical-like EGFR mutations, and the one or more kinase inhibitors selected to treat the cancer may include first-generation EGFR TKIs, second-generation EGFR TKIs, third-generation EGFR TKIs, or EGFR TKIs specific to mutations associated with EGFR exon 20. Classical-like EGFR mutations include but are not limited to those provided in Tables 1.1-1.5, below. Classical-like EGFR mutations of the disclosure may include any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 of the mutations of Tables 1.1-1.5. Any one or more mutations of Tables 1.1-1.5 may be excluded from aspects of the disclosure. “Classical-like” EGFR mutations describe EGFR mutations that are distant from the ATP binding pocket of the EGFR protein. An example of response of cells comprising a classical-like EGFR mutation to TKIs disclosed herein is also provided. Data represent the ratio of the IC₅₀ value for a given TKI for cells with a classical-like EGFR mutation to the IC₅₀ value for cells with wildtype EGFR.

TABLE 1.1
Response of Cells Comprising Classical-Like
EGFR Mutations to First-Generation TKIs

| Mutation from Wildtype | | Erlotinib | Geftinib | AZD3759 | Sapatinib |
|---|---|---|---|---|---|
| Classical-Like | E709K L858R | 0.1459391 | 0.0884749 | 0.022018 | 0.02084 |
| Classical-Like | S784F | 0.4917344 | 0.2550193 | 0.0701957 | 0.0850804 |
| Classical-Like | L833F | 0.087518 | 0.2003861 | 0.1248701 | 0.0066194 |
| Classical-Like | L833V | 0.0534978 | 0.0482606 | 0.1488175 | 0.0085935 |
| Classical-Like | L858R/V834L | 0.027809 | 0.0591583 | 0.0022409 | 0.0018892 |
| Classical-Like | Ex19del | 0.009386 | 0.020973 | 0.0001195 | 0.0005535 |
| Classical-Like | L858R | 0.0710063 | 0.2266602 | 0.0058505 | 0.0006817 |
| Classical-Like | L858R S784F | 0.0993771 | 0.168166 | 0.0154604 | 0.0666362 |
| Classical-Like | S720P | 0.0593675 | 0.0093597 | 0.0183414 | 0.004664 |
| Classical-Like | T725M | 0.1742693 | 0.3178958 | 0.1058871 | 0.0066701 |
| Classical-Like | K754E | 0.2430522 | 0.2700193 | 0.0465127 | 0.0030698 |
| Classical-Like | L861R | 0.3446694 | 0.4359073 | 0.037042 | 0.002811 |
| Classical-Like | L861Q | 0.5041861 | 0.3498263 | 0.0892397 | 0.0126479 |
| Classical-Like | S811F | 0.0444058 | 0.0841506 | 0.0064226 | 0.0065495 |
| Classical-Like | A763insFQEA | 0.423677 | 0.1749035 | 0.022249 | 0.3136779 |
| Classical-Like | A763insLQEA | 0.4453761 | 0.2283591 | 0.0290536 | 0.0042259 |
| Classical-Like | D761N | 0.1133685 | 0.406834 | 0.6757138 | 0.0018137 |

TABLE 1.2
Response of Cells Comprising Classical-Like
EGFR Mutations to Second-Generation TKIs

| | | | | | Tarlox- |
|---|---|---|---|---|---|
| Mutation from Wildtype | | Afatinib | Dacomitinib | Neratinib | TKI |
| Classical-Like | E709K L858R | 0.0046549 | 0.0079854 | 0.0377962 | 0.0057923 |
| Classical-Like | S784F | 0.0556846 | 0.089971 | 0.528502 | 0.0181567 |
| Classical-Like | L833F | 0.0334908 | 0.1863075 | 0.3250372 | 0.0788672 |
| Classical-Like | L833V | 0.0067281 | 0.0627202 | 0.293913 | 0.0031521 |
| Classical-Like | L858R/V834L | 0.0110367 | 0.0093135 | 0.0395658 | 0.0025708 |
| Classical-Like | Ex19del | 0.0084585 | 0.0058318 | 0.2591429 | 0.0142672 |
| Classical-Like | L858R | 0.0384871 | 0.0021589 | 0.1098742 | 0.1181486 |
| Classical-Like | L858R S784F | 0.08996 | 0.0644768 | 0.0520769 | 0.0016433 |
| Classical-Like | S720P | 0.059398 | 0.0387929 | 0.105408 | 0.0241169 |
| Classical-Like | T725M | 0.0057407 | 0.0306218 | 0.0852289 | 0.0048605 |
| Classical-Like | K754E | 0.0073292 | 0.0323141 | 0.0658719 | 0.0072302 |
| Classical-Like | L861R | 0.0040349 | 0.001653 | 0.006264 | 0.019255 |
| Classical-Like | L861Q | 0.0304975 | 0.0020644 | 0.0172382 | 0.0534572 |
| Classical-Like | S811F | 0.0203841 | 0.1672593 | 0.3394964 | 0.111754 |
| Classical-Like | A763insFQEA | 0.0729505 | 0.0378993 | 0.0656885 | 0.0546305 |
| Classical-Like | A763insLQEA | 0.0045222 | 0.0014839 | 0.0244176 | 0.0268983 |
| Classical-Like | D761N | 0.0026862 | 0.0008794 | 0.0910975 | 0.1318737 |

TABLE 1.3
Response of Cells Comprising Classical-Like EGFR Mutations to Third-
Generation TKIs

| Mutation from | | | | | | | |
|---|---|---|---|---|---|---|---|
| Wildtype | | Osimertinib | Nazartinib | Olmutinib | Rocelitinib | Naquotinib | Lazertinib |
| Classical- | E709K | 0.0544914 | 0.0199203 | 0.0160947 | 0.0462334 | 0.0818045 | 0.0006261 |
| Like | L858R | | | | | | |
| Classical- | S784F | 0.0023854 | 0.031072 | 0.1399754 | 0.0517571 | 0.3727228 | 0.2450393 |
| Like | | | | | | | |
| Classical- | L833F | 0.0705933 | 0.0583985 | 0.1288572 | 0.0932517 | 0.0305396 | 0.0862502 |
| Like | | | | | | | |
| Classical- | L833V | 0.0341488 | 0.0027386 | 0.0380347 | 0.0891452 | 0.0247772 | 0.0061324 |
| Like | | | | | | | |
| Classical- | L858R/ | 0.0014656 | 0.0112311 | 0.0067158 | 0.0251887 | 0.0533886 | 0.0037676 |
| Like | V834L | | | | | | |
| Classical- | Ex19del | 0.0002543 | 0.0005409 | 0.0008432 | 0.0012438 | 0.0060354 | |
| Like | | | | | | | |
| Classical- | L858R | 0.0301277 | 0.0170002 | 0.0171619 | 0.0329335 | 0.0786349 | 0.037018 |
| Like | | | | | | | |
| Classical- | L858R | 0.1133188 | 0.0357712 | 0.0456909 | 0.0995751 | 0.1920545 | 0.0245629 |
| Like | S784F | | | | | | |
| Classical- | S720P | 0.0020851 | 0.0056185 | 0.0033313 | 0.0144993 | 0.0339678 | 0.0156796 |
| Like | | | | | | | |
| Classical- | T725M | 0.3286872 | 0.0454064 | 0.0366366 | 0.2740315 | 0.5761139 | 0.0044659 |
| Like | | | | | | | |
| Classical- | K754E | 0.0088204 | 0.0474859 | 0.0088379 | 0.0733817 | 0.0490099 | 0.138475 |
| Like | | | | | | | |
| Classical- | L861R | 0.0455805 | 0.0112057 | 0.0293732 | 0.0320895 | 0.0905446 | 0.0509143 |
| Like | | | | | | | |
| Classical- | L861Q | 0.1898789 | 0.0386104 | 0.0504819 | 0.1507145 | 0.1616088 | 0.0562206 |
| Like | | | | | | | |
| Classical- | S811F | 0.0035607 | 0.0011504 | 0.0307037 | 0.0202239 | 0.0351 | 0.0115762 |
| Like | | | | | | | |
| Classical- | A763ins | 0.1162442 | 0.0567571 | 0.027529 | 0.1818137 | 0.1912376 | 0.0379707 |
| Like | FQEA | | | | | | |
| Classical- | A763ins | 0.0626132 | 0.0550668 | 0.0748344 | 0.1702824 | 0.3419307 | 0.1304013 |
| Like | LQEA | | | | | | |
| Classical- | D761N | 0.0097663 | 0.0009686 | 0.1654406 | 0.5164709 | 0.1966337 | 0.0195742 |
| Like | | | | | | | |

TABLE 1.4
Response of Cells Comprising Classical-Like
EGFR Mutations to Ex20ins-Specific TKIs

| | | | | TAK-788 |
|---|---|---|---|---|
| Mutation from Wildtype | | TAS 6417 | AZ5104 | (mobocertinib) |
| Classical-Like | E709K L858R | 0.0026071 | 0.1151302 | 0.0083555 |
| Classical-Like | S784F | 0.0101497 | 0.0142845 | 0.0148677 |
| Classical-Like | L833F | 0.0688595 | 0.728351 | 1.2333901 |
| Classical-Like | L833V | 0.0311059 | 0.444648 | 0.1366951 |
| Classical-Like | L858R/V834L | 0.0005227 | 0.4037608 | 0.0060557 |
| Classical-Like | Ex19del | 0.0014896 | 0.0303452 | 0.0285883 |
| Classical-Like | L858R | 0.0039179 | 0.1964609 | 0.0472005 |
| Classical-Like | L858R S784F | 0.0014228 | 0.0429026 | 0.001484 |
| Classical-Like | S720P | 0.0103477 | 0.0098409 | 0.1110165 |
| Classical-Like | T725M | 0.0257189 | 0.9590164 | 1.0821124 |
| Classical-Like | K754E | 0.0044745 | 0.0187097 | 0.1738444 |
| Classical-Like | L861R | 0.0298371 | 0.2335391 | 0.3101533 |
| Classical-Like | L861Q | 0.027609 | 0.5870781 | 0.846678 |
| Classical-Like | S811F | 0.0090265 | 0.0028709 | 0.0031952 |
| Classical-Like | A763insFQEA | 0.0049819 | 0.3966249 | 0.4730267 |
| Classical-Like | A763insLQEA | 0.005254 | 0.0302941 | 0.0337399 |
| Classical-Like | D761N | 0.2473523 | 0.3285439 | 0.3630324 |

TABLE 1.5
List of Example Classical-Like EGFR Mutations
Mutation from Wildtype

| | |
|---|---|
| Classical-Like | A702T |
| Classical-Like | A763insFQEA |
| Classical-Like | A763insLQEA |
| Classical-Like | D761N |
| Classical-Like | E709A L858R |
| Classical-Like | E709K L858R |
| Classical-Like | E746_A750del A647T |
| Classical-Like | E746_A750del L41W |
| Classical-Like | E746_A750del R451H |
| Classical-Like | Ex19del E746_A750del |
| Classical-Like | K754E |
| Classical-Like | L747_E749del A750P |
| Classical-Like | L747_T751del L861Q |
| Classical-Like | L833F |
| Classical-Like | L833V |
| Classical-Like | L858R |
| Classical-Like | L858R A289V |
| Classical-Like | L858R E709V |
| Classical-Like | L858R L833F |
| Classical-Like | L858R P100T |
| Classical-Like | L858R P848L |
| Classical-Like | L858R R108K |
| Classical-Like | L858R R324H |
| Classical-Like | L858R R324L |
| Classical-Like | L858R S784F |
| Classical-Like | L858R S784Y |
| Classical-Like | L858R T725M |
| Classical-Like | L858R V834L |
| Classical-Like | L861Q |
| Classical-Like | L861R |
| Classical-Like | S720P |
| Classical-Like | S784F |
| Classical-Like | S811F |
| Classical-Like | T725M |

Thus, in some embodiments, disclosed are methods for treating a subject for lung cancer (e.g., NSCLC), the method comprising administering an effective amount of one or more EGFR inhibitors (e.g., erlotinib, gefitinib, AZD3759, sapatinib, afatinib, dacomitinib, neratinib, tarlox-TKI, tarloxotinib, TAS6417 (CLN-081), AZ5104, TAK-788 (mobocertinib), osimertinib, nazartinib, olmutinib, rocelitinib, naquotinib, lazertinib, or a combination thereof) to a subject determined, from analysis of tumor DNA from the subject, to have an EGFR mutation, wherein the EGFR mutation is a classical-like mutation. In some embodiments, the classical-like EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M. Also disclosed are methods for treating a subject for lung cancer, the method comprising: (a) detecting an EGFR mutation in tumor DNA from the subject, wherein the EGFR mutation is a classical-like mutation; and (b) administering an effective amount of one or more EGFR inhibitors (e.g., erlotinib, gefitinib, AZD3759, sapatinib, afatinib, dacomitinib, neratinib, tarlox-TKI, tarloxotinib, TAS6417 (CLN-081), AZ5104, TAK-788 (mobocertinib), osimertinib, nazartinib, olmutinib, rocelitinib, naquotinib, lazertinib, or a combination thereof) to the subject. In some aspects, the classical-like EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M. Any one or more of the preceeding EGFR mutations may be excluded from aspects of the present disclosure.

In some embodiments, the method comprises administering erlotinib to the subject. In some embodiments, the method comprises administering gefitinib to the subject. In some embodiments, the method comprises administering AZD3759 to the subject. In some embodiments, the method comprises administering sapatinib to the subject. In some embodiments, the method comprises administering afatinib to the subject. In some embodiments, the method comprises administering dacomitinib to the subject. In some embodiments, the method comprises administering neratinib to the subject. In some embodiments, the method comprises administering tarlox-TKI to the subject. In some embodiments, the method comprises administering tarloxotinib to the subject. In some embodiments, the method comprises administering TAS6417 (CLN-081) to the subject. In some embodiments, the method comprises administering AZ5104 to the subject. In some embodiments, the method comprises administering TAK-788 (mobocertinib) to the subject. In some embodiments, the method comprises administering osimertinib to the subject. In some embodiments, the method comprises administering nazartinib to the subject. In some embodiments, the method comprises administering olmutinib to the subject. In some embodiments, the method comprises administering rocelitinib to the subject. In some embodiments, the method comprises administering naquotinib to the subject. In some embodiments, the method comprises administering lazertinib to the subject.

In some embodiments, the EGFR mutation is A702T. In some embodiments, the EGFR mutation is A763insFQEA. In some embodiments, the EGFR mutation is A763insLQEA. In some embodiments, the EGFR mutation is D761N. In some embodiments, the EGFR mutation is E709A L858R. In some embodiments, the EGFR mutation is E709K L858R. In some embodiments, the EGFR mutation is E746_A750del A647T. In some embodiments, the EGFR mutation is E746_A750del L41W. In some embodiments, the EGFR mutation is E746_A750del R451H. In some embodiments, the EGFR mutation is Ex19del E746_A750del. In some embodiments, the EGFR mutation is K754E. In some embodiments, the EGFR mutation is L747_E749del A750P. In some embodiments, the EGFR mutation is L747_T751del L861Q. In some embodiments, the EGFR mutation is L833F. In some embodiments, the EGFR mutation is L833V. In some embodiments, the EGFR mutation is L858R. In some embodiments, the EGFR mutation is L858R A289V. In some embodiments, the EGFR mutation is L858R E709V. In some embodiments, the EGFR mutation is L858R L833F. In some embodiments, the EGFR mutation is L858R P100T. In some embodiments, the EGFR mutation is L858R P848L. In some embodiments, the EGFR mutation is L858R R108K. In some embodiments, the EGFR mutation is L858R R324H. In some embodiments, the EGFR mutation is L858R R324L. In some embodiments, the EGFR mutation is L858R S784F. In some embodiments, the EGFR mutation is L858R S784Y. In some embodiments, the EGFR mutation is L858R T725M. In some embodiments, the EGFR mutation is L858R V834L. In some embodiments, the EGFR mutation is L861Q. In some embodiments, the EGFR mutation is L861R. In some embodiments, the EGFR mutation is S720P. In some embodiments, the EGFR mutation is S784F. In some embodiments, the EGFR mutation is S811F. In some embodiments, the EGFR mutation is T725M.

In some embodiments, the subject was previously treated with a cancer therapy. In some embodiments, the cancer therapy comprised chemotherapy. In some embodiments, the subject was determined to be resistant to the cancer therapy.

### B. Exon 20 Loop Insertion Mutations

In some cases, the subject having cancer (e.g., lung cancer such as non-small cell lung cancer) is determined to have one or more Exon 20 loop insertion (ex20ins) EGFR mutations, and the kinase inhibitor selected may include second-generation TKIs or TKIs specific to mutations associated with EGFR exon 20. In some aspects, an Ex20ins EGFR mutation is an Ex20ins near-loop (NL) mutation. Ex20ins EGFR mutations include but are not limited to those provided in Tables 2.1-2.5, below. “Ex20ins” EGFR mutations describe EGFR mutations that are insertion mutations in exon 20 of the EGFR gene, including mutations at the c-terminal of the α-c-helix of the EGFR protein. An example of response of cells comprising an ex20ins EGFR mutation to TKIs disclosed herein is also provided. Data represent the ratio of the IC₅₀ value for a given TKI for cells with an ex20ins EGFR mutation to the IC₅₀ value for cells with wildtype EGFR.

TABLE 2.1
Response of Cells Comprising Ex20ins EGFR Mutations to First-Generation TKIs

| Mutation from Wildtype | | Erlotinib | Geftinib | AZD3759 | Sapatinib |
|---|---|---|---|---|---|
| Ex20 | A767insASV | 60.27911 | 30.47104 | 4.153994 | 2.232834 |
| Ex20 | S768dupSVD | 11.3632 | 97.14479 | 3.074753 | 2.666182 |
| Ex20 | S768dupSVD V769M | 9.779588 | 28.95753 | 1.659288 | 1.596056 |
| Ex20 | D770insNPG | 16.69741 | 26.32239 | 3.835419 | 5.021914 |
| Ex20 | H773insNPH | 5.552228 | 28.95753 | 1.561437 | 1.264609 |
| Ex20 | N771dupN | 6.068507 | 28.95753 | 1.566891 | 1.240321 |
| Ex20 | N771dupN G724S | 6.115237 | 28.95753 | 1.258004 | 1.102849 |

TABLE 2.2
Response of Cells Comprising Ex20ins EGFR
Mutations to Second-Generation TKIs

| Mutation from Wildtype | | Afatinib | Dacomitinib | Neratinib | Tarlox-TKI |
|---|---|---|---|---|---|
| Ex20 | A767insASV | 4.070091 | 3.056511 | 3.51839 | 0.994519 |
| Ex20 | S768dupSVD | 0.783519 | 0.882037 | 0.930406 | 0.306461 |
| Ex20 | S768dupSVD V769M | 2.308146 | 3.096798 | 2.04937 | 0.330043 |
| Ex20 | D770insNPG | 6.25763 | 5.404491 | 27.85962 | 5.023346 |
| Ex20 | H773insNPH | 2.048306 | 1.645037 | 7.716368 | 11.55907 |
| Ex20 | N771dupN | 1.249 | 6.261281 | 0.951522 | 7.062322 |
| Ex20 | N771dupN G724S | 1.337403 | 3.161474 | 1.920541 | 0.830085 |

TABLE 2.3
Response of Cells Comprising Ex20ins EGFR Mutations to Third-Generation
TKIs

| Mutation from | | | | | | | |
|---|---|---|---|---|---|---|---|
| Wildtype | | Osimertinib | Nazartinib | Olmutinib | Rocelitinib | Naquotinib | Lazertinib |
| Ex20 | A767insASV | 2.386147347 | 0.638174807 | 0.679238374 | 1.859285179 | 1.61039604 | 10.92828477 |
| Ex20 | S768dupSVD | 0.999490274 | 0.400822365 | 0.553310542 | 0.864083604 | 1.134900495 | 4.026871401 |
| Ex20 | S768dupSVD | 0.69286477 | 0.326169666 | 0.537864918 | 0.487203199 | 1.960148515 | 2.831460478 |
| | V769M | | | | | | |
| Ex20 | D770insNPG | 1.334199898 | 0.529023136 | 0.310042941 | 0.685953512 | 1.296287129 | 4.930378642 |
| Ex20 | H773insNPH | 0.931943735 | 0.424241645 | 0.427415865 | 0.873781555 | 1.07710396 | 5.101727447 |
| Ex20 | N771dupN | 0.669571979 | 0.371028278 | 0.420758297 | 0.694626343 | 3.012128713 | |
| Ex20 | N771dupN G724S | 1.18613342 | 0.338354756 | 0.416497453 | 0.857035741 | 2.780940594 | |

TABLE 2.4
Response of Cells Comprising Ex20ins EGFR
Mutations to Ex20ins-Specific TKIs

| | | | | TAK-788 |
|---|---|---|---|---|
| Mutation from Wildtype | | TAS 6417 | AZ5104 | (mobocertinib) |
| Ex20 | A767insASV | 0.13586558 | 4.660559306 | 1.553208404 |
| Ex20 | S768dupSVD | 0.057727088 | 1.729893925 | 0.251470755 |
| Ex20 | S768dupSVD | 0.026786151 | 0.386441659 | 0.544622374 |
| | V769M | | | |
| Ex20 | D770insNPG | 0.016796334 | 1.975506268 | 1.211470755 |
| Ex20 | H773insNPH | 0.184338086 | 3.996721311 | 0.295184554 |
| Ex20 | N771dupN | 0.051342159 | 0.604435873 | 0.85076661 |
| Ex20 | N771dupN G724S | 0.051132383 | 1.826133076 | 1.553208404 |

TABLE 2.5
List of Example Exon 20 Loop Insertion EGFR Mutations
Mutation from Wildtype

| | |
|---|---|
| Exon 20 Loop Insertion (Far-loop) | H773_V774 insNPH |
| Exon 20 Loop Insertion (Far-loop) | H773_V774 insAH |
| Exon 20 Loop Insertion (Far-loop) | H773dupH |
| Exon 20 Loop Insertion (Far-loop) | V774_C775 insHV |
| Exon 20 Loop Insertion (Far-loop) | V774_C775 insPR |
| Exon 20 Loop Insertion (Near-loop) | A767_V769dupASV |
| Exon 20 Loop Insertion (Near-loop) | A767_S768insTLA |
| Exon 20 Loop Insertion (Near-loop) | S768_D770dupSVD |
| Exon 20 Loop Insertion (Near-loop) | S768_D770dupSVD L858Q |
| Exon 20 Loop Insertion (Near-loop) | S768_D770dupSVD R958H |
| Exon 20 Loop Insertion (Near-loop) | S768_D770dupSVD V769M |
| Exon 20 Loop Insertion (Near-loop) | V769_D770insASV |
| Exon 20 Loop Insertion (Near-loop) | V769_D770insGSV |
| Exon 20 Loop Insertion (Near-loop) | V769_D770insGVV |
| Exon 20 Loop Insertion (Near-loop) | V769_D770insMASVD |
| Exon 20 Loop Insertion (Near-loop) | D770_N771insNPG |
| Exon 20 Loop Insertion (Near-loop) | D770_N771insSVD |
| Exon 20 Loop Insertion (Near-loop) | D770del insGY |
| Exon 20 Loop Insertion (Near-loop) | D770_N771 insG |
| Exon 20 Loop Insertion (Near-loop) | D770_N771 insY H773Y |
| Exon 20 Loop Insertion (Near-loop) | N771dupN |
| Exon 20 Loop Insertion (Near-loop) | N771dupN G724S |
| Exon 20 Loop Insertion (Near-loop) | N771_P772insHH |
| Exon 20 Loop Insertion (Near-loop) | N771_P772insSVDNR |
| Exon 20 Loop Insertion (Near-loop) | P772_H773insDNP |

Thus, in some embodiments, disclosed are methods for treating a subject for lung cancer, the method comprising administering an effective amount of one or more second-generation or Exon20 loop insertion-specific EGFR inhibitors (e.g., afatinib, dacomitinib, neratinib, tarlox-TKI, tarloxotinib, TAS6417 (CLN-081), AZ5104, TAK-788 (mobocertinib), or a combination thereof) to a subject determined, from analysis of tumor DNA from the subject, to have an EGFR mutation, wherein the EGFR mutation is an Exon20 near-loop insertion (ex20ins-NL) mutation. In some aspects, the exon20ins-NL mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP. Also disclosed are methods for treating a subject for lung cancer, the method comprising: (a) detecting an EGFR mutation in tumor DNA from the subject, wherein the EGFR mutation is an exon20ins-NL mutation; and (b) administering an effective amount of one or more second-generation or Exon20 loop insertion-specific EGFR inhibitors (e.g., afatinib, dacomitinib, neratinib, tarlox-TKI, tarloxotinib, TAS6417 (CLN-081), AZ5104, TAK-788 (mobocertinib), or a combination thereof) to the subject. In some aspects, the ex20ins-NL mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP. Further disclosed are methods comprising administering an EGFR inhibitor to a subject determined, from analysis of tumor DNA from the subject, to have an Exon20 near-loop insertion EGFR mutation.

In some embodiments, the method comprises administering afatinib to the subject. In some embodiments, the method comprises administering dacomitinib to the subject. In some embodiments, the method comprises administering neratinib to the subject. In some embodiments, the method comprises administering tarlox-TKI to the subject. In some embodiments, the method comprises administering tarloxotinib to the subject. In some embodiments, the method comprises administering TAS6417 (CLN-081) to the subject. In some embodiments, the method comprises administering AZ5104 to the subject. In some embodiments, the method comprises administering TAK-788 (mobocertinib) to the subject.

In some embodiments, the EGFR mutation is A767_V769dupASV. In some embodiments, the EGFR mutation is A767_S768insTLA. In some embodiments, the EGFR mutation is S768_D770dupSVD. In some embodiments, the EGFR mutation is S768_D770dupSVD L858Q. In some embodiments, the EGFR mutation is S768_D770dupSVD R958H. In some embodiments, the EGFR mutation is S768_D770dupSVD V769M. In some embodiments, the EGFR mutation is V769_D770insASV. In some embodiments, the EGFR mutation is V769_D770insGSV. In some embodiments, the EGFR mutation is V769_D770insGVV. In some embodiments, the EGFR mutation is V769_D770insMASVD. In some embodiments, the EGFR mutation is D770_N771insNPG. In some embodiments, the EGFR mutation is D770_N771insSVD. In some embodiments, the EGFR mutation is D770del insGY. In some embodiments, the EGFR mutation is D770_N771 insG. In some embodiments, the EGFR mutation is D770_N771 insY H773Y. In some embodiments, the EGFR mutation is N771dupN. In some embodiments, the EGFR mutation is N771dupN G724S. In some embodiments, the EGFR mutation is N771_P772insHH. In some embodiments, the EGFR mutation is N771_P772insSVDNR. In some embodiments, the EGFR mutation is P772_H773insDNP.

In some embodiments, the subject was previously treated with a cancer therapy. In some embodiments, the cancer therapy comprised erlotinib, gefitinib, AZD3759, or sapatinib. In some embodiments, the cancer therapy comprised osimertinib, nazartinib, olmutinib, rocelitinib, naquotinib, lazertinib. In some embodiments, the cancer therapy comprised chemotherapy. In some embodiments, the subject was determined to be resistant to the cancer therapy.

### C. T790M-Like Mutations

In some cases, the subject having cancer (e.g., lung cancer) is determined to have one or more T790M-like-EGFR mutations. “T790M-like” EGFR mutants contain at least one mutation in the hydrophobic cleft; the addition of one or more known resistance mutations can reduce sensitivity to classical EGFR TKIs. In some cases, the subject having cancer is determined to have one or more T790M-like-EGFR mutations but no detected resistance mutations (i.e., C797S^{37,38}, L718X^{18,24}, or L792H^{23,24}, which confer resistance to classical EGFR TKIs), referred to herein as “T790M-like-3S” mutants, and the kinase inhibitor selected may include third-generation TKIs, TKIs specific to mutations associated with EGFR exon 20, ALK inhibitors, and/or PKC inhibitors. T790M-like-3S EGFR mutations include but are not limited to those provided in Tables 3.1-3.5, below. T790M-like-3S EGFR mutations may describe EGFR mutations that are present in the hydrophobic core of the EGFR protein. An example of response of cells comprising a T790M-like-3S EGFR mutation to TKIs disclosed herein is also provided. Data represent the ratio of the IC₅₀ value for a given TKI for cells with a T790M-like-3S EGFR mutation to the IC₅₀ value for cells with wildtype EGFR.

TABLE 3.1
Response of Cells Comprising T790M-like-3S EGFR Mutations to First-Generation TKIs

| Mutation from Wildtype | | Erlotinib | Geftinib | AZD3759 | Sapatinib |
|---|---|---|---|---|---|
| T790M-like-3S | L747_K754del insATSPE | 18.02467657 | 28.95752896 | 4.604106513 | 7.978451424 |
| T790M-like-3S | T790M | 35.93675132 | 21.64864865 | 21.82003208 | 70.50220964 |
| T790M-like-3S | L858R/T790M | 35.93675132 | 28.95752896 | 6.744311838 | 38.8604821 |
| T790M-like-3S | Ex19del T790M | 17.87370879 | 23.54633205 | 12.68206288 | 10.73228999 |
| T790M-like-3S | G719A T790M | 35.93675132 | 30.43050193 | 9.624639076 | 54.78451424 |
| T790M-like-3S | G719S T790M | 29.58064447 | 28.95752896 | 8.020532563 | 54.78451424 |
| T790M-like-3S | S768I/T790M | 17.76474605 | 28.95752896 | 7.218479307 | 54.78451424 |
| T790M-like-3S | L858R T790M V843I | 35.93675132 | 28.95752896 | 9.624639076 | 48.99744339 |
| T790M-like-3S | Ex19del/T790M/G724S | 19.02491016 | 14.13320463 | 4.644850818 | 26.15230095 |
| T790M-like-3S | L858R T790M L792H | 25.48994609 | 7.851351351 | 9.624639076 | 54.78451424 |
| T790M-like-3S | Ex19del T790M L718V | 17.96837566 | 28.95752896 | 4.812319538 | 27.39225712 |

TABLE 3.2
Response of Cells Comprising T790M-like-3S EGFR Mutations to Second-Generation TKIs

| Mutation from Wildtype | | Afatinib | Dacomitinib | Neratinib | Tarlox-TKI |
|---|---|---|---|---|---|
| T790M-like-3S | L747_K754del insATSPE | 1.185855609 | 5.465191233 | 5.935858035 | 4.873934227 |
| T790M-like-3S | T790M | 2.685592191 | 6.060711216 | 1.80688971 | 4.920422249 |
| T790M-like-3S | L858R/T790M | 10.58514523 | 10.00375967 | 12.35946961 | 11.95290296 |
| T790M-like-3S | Ex19del T790M | 4.025855504 | 4.87052283 | 34.1337583 | 14.38083638 |
| T790M-like-3S | G719A T790M | 1.313638339 | 0.919531317 | 0.968059587 | 0.677831912 |
| T790M-like-3S | G719S T790M | 0.802993044 | 3.776482596 | 0.442341286 | 3.949248883 |
| T790M-like-3S | S768I/T790M | 0.753525363 | 1.281950465 | 0.701397839 | 60.18879415 |
| T790M-like-3S | L858R T790M V843I | 10.47147969 | 20.05801461 | 69.55982948 | 70.97036135 |
| T790M-like-3S | Ex19del/T790M/G724S | 1.039886234 | 9.950042974 | 5.270893229 | 4.197117337 |
| T790M-like-3S | L858R T790M L792H | 21.13029678 | 8.291261818 | 19.89888123 | 5.197929354 |
| T790M-like-3S | Ex19del T790M L718V | 6.885918754 | 89.61645896 | 40.36879151 | 154.4051969 |

TABLE 3.3
Response of Cells Comprising T790M-like-3S EGFR Mutations to Third-
Generation TKIs

| Mutation from Wildtype | | Osimertinib | Nazartinib | Olmutinib | Rocelitinib | Naquotinib | Lazertinib |
|---|---|---|---|---|---|---|---|
| T790M- | L747_K754del | 0.002805116 | 0.001194859 | 0.000135658 | 0.000690777 | 0.00432995 | 2.781085325 |
| like-3S | insATSPE | | | | | | |
| T790M- | T790M | 0.242885669 | 0.116627517 | 0.04610765 | 0.077372167 | 0.403712847 | 0.251718723 |
| like-3S | | | | | | | |
| T790M- | L858R/T790M | 0.012833659 | 0.019570692 | 0.000793583 | 0.003154212 | 0.013975252 | 0.033597976 |
| like-3S | | | | | | | |
| T790M- | Ex19del T790M | 0.019518521 | 0.012647153 | 0.000405939 | 0.003577809 | 0.009603953 | |
| like-3S | | | | | | | |
| T790M- | G719A T790M | 0.346671348 | 0.026527894 | 0.011396718 | 0.009756519 | 0.087022272 | 0.001079567 |
| like-3S | | | | | | | |
| T790M- | G719S T790M | 0.928717492 | 0.048112982 | 0.024531071 | 0.041664872 | 0.140940594 | 0.003464317 |
| like-3S | | | | | | | |
| T790M- | S768I/T790M | 0.021658686 | 0.019701794 | 0.021497297 | 0.019414344 | 0.142574257 | 0.523625894 |
| like-3S | | | | | | | |
| T790M- | L858R T790M | 0.042344831 | 0.024976864 | 0.000771013 | 0.003553112 | 0.015185644 | 0.066119351 |
| like-3S | V843I | | | | | | |
| T790M- | Ex19del/ | 0.146511281 | 0.061174807 | 0.005075064 | 0.02974007 | 0.028492574 | 0.02983947 |
| like-3S | T790M/G724S | | | | | | |
| T790M- | L858R T790M | 0.268557588 | 0.028843193 | 0.004757169 | 0.372731992 | 0.035294559 | 0.000500855 |
| like-3S | L792H | | | | | | |
| T790M- | Ex19del T790M | 1.320969314 | 0.259383033 | 0.021647082 | 0.052581855 | 0.029938119 | 0.466672483 |
| like-3S | L718V | | | | | | |

TABLE 3.4
Response of Cells Comprising T790M-like-3S
EGFR Mutations to Ex20ins-Specific TKIs

| | | | | TAK-788 |
|---|---|---|---|---|
| Mutation from Wildtype | | TAS 6417 | AZ5104 | (mobocertinib) |
| T790M-like-3S | L747_K754del insATSPE | 0.00046613 | | |
| T790M-like-3S | T790M | 0.66712831 | 1.125458052 | 1.314593981 |
| T790M-like-3S | L858R/T790M | 0.019633401 | 0.177666345 | 0.756501988 |
| T790M-like-3S | Ex19del T790M | 0.001513035 | 0.183702989 | 0.239125497 |
| T790M-like-3S | G719A T790M | 0.012955193 | 0.166740598 | 0.486428166 |
| T790M-like-3S | G719S T790M | 0.032 | 0.183924783 | 1.301987507 |
| T790M-like-3S | S768I/T790M | 0.115254582 | 1.619093539 | 10.82453152 |
| T790M-like-3S | L858R T790M V843I | 0.073360489 | 0.693442623 | 1.649063032 |
| T790M-like-3S | Ex19del/T790M/G724S | 0.199678208 | 1.67340405 | 3.119704713 |
| T790M-like-3S | L858R T790M L792H | 9.53564E−05 | 0.58707811 | 3.32629188 |
| T790M-like-3S | Ex19del T790M L718V | 0.002883605 | 0.704146577 | 3.032027257 |

TABLE 3.5
List of Example T790M-like-3S EGFR Mutations
Mutation from Wildtype

| | |
|---|---|
| T790M-like-3S | Ex19del T790M |
| T790M-like-3S | Ex19del T790M L718V |
| T790M-like-3S | Ex19del T790M G724S |
| T790M-like-3S | G719A T790M |
| T790M-like-3S | G719S T790M |
| T790M-like-3S | H773R T790M |
| T790M-like-3S | I744_E749del insMKK |
| T790M-like-3S | L747_K754 delinsATSPE |
| T790M-like-3S | L858R T790M L792H |
| T790M-like-3S | L858R T790M V843I |
| T790M-like-3S | L858R T790M |
| T790M-like-3S | S768I T790M |
| T790M-like-3S | T790M |

Thus, in some embodiments, disclosed are methods for treating a subject for lung cancer, the method comprising administering an effective amount of one or more third-generation EGFR inhibitors, PKC inhibitors, and/or ALK inhibitors (e.g., osimertinib, nazartinib, olmutinib, rocelitinib, naquotinib, lazertinib, brigatinib, AZD3463, ruboxistaurin, midostaurin, sotrastaurin, or a combination thereof) to a subject determined, from analysis of tumor DNA from the subject, to have an EGFR mutation, wherein the EGFR mutation is a T790M-like-3S mutation. In some aspects, the T790M-like-3S mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M. Also disclosed are methods for treating a subject for lung cancer, the method comprising: (a) detecting a EGFR mutation in tumor DNA from the subject, wherein the EGFR mutation is a T790M-like-3S mutation; and (b) administering an effective amount of one or more third-generation EGFR inhibitors, PKC inhibitors, and/or ALK inhibitors (e.g., osimertinib, nazartinib, olmutinib, rocelitinib, naquotinib, lazertinib, brigatinib, AZD3463, ruboxistaurin, midostaurin, sotrastaurin, or a combination thereof) to the subject. Further disclosed are methods comprising administering an EGFR inhibitor to a subject determined, from analysis of tumor DNA from the subject, to have a T790M-like-3S EGFR mutation.

In some embodiments, the method comprises administering osimertinib to the subject. In some embodiments, the method comprises administering nazartinib to the subject. In some embodiments, the method comprises administering olmutinib to the subject. In some embodiments, the method comprises administering rocelitinib to the subject. In some embodiments, the method comprises administering naquotinib to the subject. In some embodiments, the method comprises administering lazertinib to the subject. In some embodiments, the method comprises administering brigatinib to the subject. In some embodiments, the method comprises administering AZD3463 to the subject. In some embodiments, the method comprises administering ruboxistaurin to the subject. In some embodiments, the method comprises administering midostaurin to the subject. In some embodiments, the method comprises administering sotrastaurin to the subject.

In some embodiments, the EGFR mutation is Ex19del T790M. In some embodiments, the EGFR mutation is Ex19del T790M L718V. In some embodiments, the EGFR mutation is Ex19del T790M G724S. In some embodiments, the EGFR mutation is G719A T790M. In some embodiments, the EGFR mutation is G719S T790M. In some embodiments, the EGFR mutation is H773R T790M. In some embodiments, the EGFR mutation is I744_E749del insMKK. In some embodiments, the EGFR mutation is L747_K754 delinsATSPE. In some embodiments, the EGFR mutation is L858R T790M L792H. In some embodiments, the EGFR mutation is L858R T790M V843I. In some embodiments, the EGFR mutation is L858R T790M. In some embodiments, the EGFR mutation is S768I T790M. In some embodiments, the EGFR mutation is T790M.

In some embodiments, the subject was previously treated with a cancer therapy. In some embodiments, the cancer therapy comprised erlotinib, gefitinib, AZD3759, or sapatinib. In some embodiments, the cancer therapy comprised chemotherapy. In some embodiments, the subject was determined to be resistant to the cancer therapy.

In some cases, the subject having cancer (e.g., lung cancer) is determined to have one or more T790M-like EGFR mutations and one or more resistance mutations (i.e., C797S^{37,38}, L718X^{18,24}, or L792H^{23,24}, which confer resistance to classical EGFR TKIs), referred to herein as “T790M-like-3R” mutants, and the kinase inhibitor selected may include ALK inhibitors or PKC inhibitors. T790M-like-3R EGFR mutations include but are not limited to those provided in Tables 4.1-4.5, below. T790M-like-3R EGFR mutations may describe EGFR mutations that comprise a mutation in the hydrophobic core of the EGFR protein (e.g., T790M) and also a mutation outside the hydrophobic core of the EGFR protein (e.g., C797S, L718X, or L792H). An example of response of cells comprising a T790M-like-3R EGFR mutation to TKIs disclosed herein is also provided. Data represent the ratio of the IC₅₀ value for a given TKI for cells with a T790M-like-3R EGFR mutation to the IC₅₀ value for cells with wildtype EGFR.

TABLE 4.1
Response of Cells Comprising T790M-like-3R EGFR Mutations to First-Generation TKIs

| Mutation from Wildtype | | Erlotinib | Geftinib | AZD3759 | Sapatinib |
|---|---|---|---|---|---|
| T790M-like-3R | L718Q T790M | 35.93675132 | 9.378378378 | 9.624639076 | 54.78451424 |
| T790M-like-3R | G724S T790M | 35.93675132 | 14.36486486 | 7.700352903 | 48.61943024 |
| T790M-like-3R | L858R/T790M/L718Q | 17.96837566 | 28.95752896 | 4.812319538 | 27.39225712 |
| T790M-like-3R | L858R T790M L718V | 17.96837566 | 28.95752896 | 4.812319538 | 41.48466034 |
| T790M-like-3R | Ex19del/T790M/L792H | 96.45311452 | 11.48069498 | 9.624639076 | 451.6707999 |
| T790M-like-3R | L858R/T790M/C797S | 35.93675132 | 5.501930502 | 9.624639076 | 66.82065376 |
| T790M-like-3R | Ex19del T790M C797S | 35.93675132 | 28.95752896 | 9.624639076 | 17.12928963 |

TABLE 4.2
Response of Cells Comprising T790M-like-3R EGFR Mutations to Second-Generation TKIs

| Mutation from Wildtype | | Afatinib | Dacomitinib | Neratinib | Tarlox-TKI |
|---|---|---|---|---|---|
| T790M-like-3R | L718Q T790M | 2.19438013 | 4.784056725 | 52.93942699 | 16.45960211 |
| T790M-like-3R | G724S T790M | 3.556093454 | 12.05146111 | 3.239317934 | 1.040600893 |
| T790M-like-3R | L858R/T790M/L718Q | 42.44369606 | 45.77782553 | 15.90661247 | 304.5066991 |
| T790M-like-3R | L858R T790M L718V | 14.48326668 | 20.65427589 | 8.79349658 | 151.5834348 |
| T790M-like-3R | Ex19del/T790M/L792H | 44.56426437 | 48.50666094 | 24.31892337 | 74.96954933 |
| T790M-like-3R | L858R/T790M/C797S | 61.16605978 | 41.68993876 | 41.87073461 | 20.70239545 |
| T790M-like-3R | Ex19del T790M C797S | 79.08257419 | 38.03251988 | 26.51359076 | 33.61956963 |

TABLE 4.3
Response of Cells Comprising T790M-like-3R EGFR Mutations to Third-
Generation TKIs

| Mutation from Wildtype | | Osimertinib | Nazartinib | Olmutinib | Rocelitinib | Naquotinib | Lazertinib |
|---|---|---|---|---|---|---|---|
| T790M- | L718Q T790M | 5.773640964 | 0.316966581 | 0.357877567 | 0.771782054 | 1.576485149 | 3.512999477 |
| like-3R | | | | | | | |
| T790M- | G724S T790M | 0.258066014 | 0.186714653 | 0.019849539 | 0.090214946 | 0.139480198 | 26.17344268 |
| like-3R | | | | | | | |
| T790M- | L858R/T790M/ | 2.316744812 | 0.771362468 | 0.619586565 | 0.833941515 | 0.681509901 | 11.8705287 |
| like-3R | L718Q | | | | | | |
| T790M- | L858R T790M | 1.066060071 | 0.659151671 | 0.875370327 | 0.806188453 | 0.641304455 | 0.540359449 |
| like-3R | L718V | | | | | | |
| T790M- | Ex19del/ | 17.42955991 | 4.292289974 | 2.217635565 | 0.754211847 | 4.634156683 | 1.104693771 |
| like-3R | T790M/L792H | | | | | | |
| T790M- | L858R/T790M/ | 20.40992294 | 2.648843445 | 4.110716687 | 1.065983504 | 32.29455446 | 23.2891293 |
| like-3R | C797S | | | | | | |
| T790M- | Ex19del T790M | 16.2156924 | 5.556990668 | 1.925625245 | 0.829785826 | 14.64109406 | 26.17344268 |
| like-3R | C797S | | | | | | |

TABLE 4.4
Response of Cells Comprising T790M-like-3R
EGFR Mutations to Ex20ins-Specific TKIs

| | | | | TAK-788 |
|---|---|---|---|---|
| Mutation from Wildtype | | TAS 6417 | AZ5104 | (mobocertinib) |
| T790M-like-3R | L718Q T790M | 0.026238493 | 26.6027001 | 43.08915389 |
| T790M-like-3R | G724S T790M | 0.169338086 | 0.427994214 | 48.31345826 |
| T790M-like-3R | L858R/T790M/L718Q | 3.054989817 | 29.86499518 | 149.165247 |
| T790M-like-3R | L858R T790M L718V | 2.010570265 | 19.66104147 | 5.795513913 |
| T790M-like-3R | Ex19del/T790M/L792H | 0.000263666 | 1.485728062 | 0.884951732 |
| T790M-like-3R | L858R/T790M/C797S | 4.897148676 | 78.1099325 | 134.6848382 |
| T790M-like-3R | Ex19del T790M C797S | 3.054989817 | 56.35486982 | 117.7853492 |

TABLE 4.5
List of Example T790M-like-3R EGFR Mutations
Mutation from Wildtype

| | |
|---|---|
| T790M-like-3R | Ex19del T790M L792H |
| T790M-like-3R | G724S T790M |
| T790M-like-3R | L718Q T790M |
| T790M-like-3R | L858R T790M C797S |
| T790M-like-3R | L858R T790M L718Q |
| T790M-like-3R | L858R T790M L718V |

Thus, in some embodiments, disclosed are methods for treating a subject for lung cancer, the method comprising administering an effective amount of one or more PKC inhibitors and/or ALK inhibitors (e.g., brigatinib, AZD3463, ruboxistaurin, midostaurin, sotrastaurin, or a combination thereof) to a subject determined, from analysis of tumor DNA from the subject, to have an EGFR mutation, wherein the EGFR mutation is a T790M-like-3R mutation. In some aspects, the T790M-like-3R mutation is Ex19del T790M C797S, Ex19del T790M L792H, G724S T790M, L718Q T790M, L858R T790M C797S, L858R T790M L718Q, or L858R T790M L718V. Further disclosed are methods comprising administering a tyrosine kinase inhibitor to a subject determined, from analysis of tumor DNA from the subject, to have a T790M-like-3R EGFR mutation, wherein the tyrosine kinase inhibitor is not an EGFR inhibitor.

Also disclosed are methods for treating a subject for lung cancer, the method comprising: (a) detecting an EGFR mutation in tumor DNA from the subject, wherein the EGFR mutation is a T790M-like-3R mutation; and (b) administering an effective amount of one or more PKC inhibitors and/or ALK inhibitors (e.g., brigatinib, AZD3463, ruboxistaurin, midostaurin, sotrastaurin, or a combination thereof) to the subject. In some aspects, the T790M-like-3R mutation is Ex19del T790M C797S, Ex19del T790M L792H, G724S T790M, L718Q T790M, L858R T790M C797S, or L858R T790M L718Q.

In some embodiments, the method comprises administering brigatinib to the subject. In some embodiments, the method comprises administering AZD3463 to the subject. In some embodiments, the method comprises administering ruboxistaurin to the subject. In some embodiments, the method comprises administering midostaurin to the subject. In some embodiments, the method comprises administering sotrastaurin to the subject.

In some embodiments, the EGFR mutation is Ex19del T790M C797S. In some embodiments, the EGFR mutation is Ex19del T790M L792H. In some embodiments, the EGFR mutation is G724S T790M. In some embodiments, the EGFR mutation is L718Q T790M. In some embodiments, the EGFR mutation is L858R T790M C797S. In some embodiments, the EGFR mutation is L858R T790M L718Q.

In some embodiments, the subject was previously treated with a cancer therapy. In some embodiments, the cancer therapy comprised erlotinib, gefitinib, AZD3759, or sapatinib. In some embodiments, the cancer therapy comprised afatinib, dacomitinib, neratinib, tarlox-TKI, tarloxotinib. In some embodiments, the cancer therapy comprised TAS6417 (CLN-081), AZ5104, or TAK-788 (mobocertinib). In some embodiments, the cancer therapy comprised osimertinib, nazartinib, olmutinib, rocelitinib, naquotinib, lazertinib. In some embodiments, the cancer therapy comprised chemotherapy. In some embodiments, the subject was determined to be resistant to the cancer therapy.

### D. PACC Mutations

In some embodiments, the subject having cancer (e.g., lung cancer) is determined to have one or more P-loop and αC-helix compressing (PACC) mutations comprising mutations spanning EGFR exons 18-21 including mutations such as G719X, L747X, S768I, L792X, and T854I and others, and the kinase inhibitor selected may include second-generation TKIs. PACC EGFR mutations include but are not limited to those provided in Tables 5.1-5.5, below. “PACC” EGFR mutations describe EGFR mutations that are present in the interior of the ATP binding pocket and/or in the c-terminal of the α-c-helix. An example of response of cells comprising a PACC EGFR mutation to TKIs disclosed herein is also provided. Data represent the ratio of the IC₅₀ value for a given TKI for cells with a PACC EGFR mutation to the IC₅₀ value for cells with wildtype EGFR.

TABLE 5.1
Response of Cells Comprising PACC EGFR Mutations to First-Generation TKIs

| Mutation from Wildtype | | Erlotinib | Geftinib | AZD3759 | Sapatinib |
|---|---|---|---|---|---|
| PACC | E709_T710del insD | 0.833373263 | 7.727799228 | 0.212255374 | 1.754382761 |
| PACC | E709K G719S | 0.560265932 | 1.992471042 | 0.363599615 | 0.011020818 |
| PACC | E709A G719S | 0.357834212 | 3.431660232 | 0.213089509 | 0.022273557 |
| PACC | E709A | 0.456037374 | 0.484749035 | 0.12409368 | 0.324324324 |
| PACC | E709K | 0.33672736 | 0.688610039 | 0.312897016 | 0.121585099 |
| PACC | L718Q | 6.111643507 | 9.353281853 | 1.696182226 | 2.030314098 |
| PACC | L718V | 2.194777192 | 24.22200772 | 0.689846006 | 1.264243974 |
| PACC | G719S | 0.228369473 | 1.516216216 | 0.011053577 | 0.199820307 |
| PACC | G719A | 0.526916219 | 4.489382239 | 0.100163673 | 0.012637513 |
| PACC | G719A L861Q | 0.552738309 | 23.72200772 | 0.074581328 | 0.046150475 |
| PACC | G719A/R776C | 0.515333014 | 2.330501931 | 0.200641643 | 0.04890431 |
| PACC | G724S | 2.306576425 | 0.538262548 | 0.011053577 | 0.140960592 |
| PACC | I740dupIPVAK | 0.339386679 | 5.661969112 | 0.089842798 | 0.008350986 |
| PACC | L747P | 1.69657403 | 2.65984556 | 0.293743985 | 0.030756026 |
| PACC | L747S | 0.410265932 | 3.566216216 | 0.081222329 | 0.005188641 |
| PACC | K757R | 0.936032343 | 5.518918919 | 0.708501765 | 0.314390066 |
| PACC | S768I | 1.569717298 | 3.303281853 | 0.14498428 | 0.188332159 |
| PACC | S768I/V769L | 3.259583134 | 3.141891892 | 0.707186397 | 0.259550767 |
| PACC | S768I V774M | 0.961667465 | 2.885907336 | 0.255534167 | 0.264791819 |
| PACC | V769L | 4.646621945 | 6.368918919 | 0.976451716 | 0.199214755 |
| PACC | V774M | 1.165548634 | 4.646138996 | 0.313923645 | 0.0557542 |
| PACC | R776H | 0.182091519 | 1.403474903 | 0.021851139 | 0.001939007 |
| PACC | R776C | 1.321993292 | 4.731660232 | 0.15217196 | 0.020115047 |
| PACC | L858R/L718V | 0.789410637 | 2.7003861 | 0.24080847 | 0.0557542 |
| PACC | L858R L718Q | 1.44070436 | 10.6023166 | 0.376547963 | 0.196146822 |
| PACC | Ex19del G724S | 5.015572592 | 2.906370656 | 4.812319538 | 27.39225712 |
| PACC | L858R/L792H | 0.143651186 | 1.828185328 | 0.031254415 | 0.012374545 |
| PACC | Ex19del/L792H | 35.02277192 | 28.95752896 | 9.624639076 | 53.47150475 |
| PACC | Ex19del G796S | 15.67681481 | 28.95752896 | 4.084055181 | 3.423484295 |
| PACC | L858R/C797S | 0.028487068 | 0.19492278 | 0.001894771 | 0.001100809 |
| PACC | Ex19del/C797S | 0.00752276 | 0.041996139 | 0.000137473 | 0.000608477 |
| PACC | Ex19del L718V | 0.310745089 | 0.253976834 | 0.041267244 | 0.080157049 |
| PACC | Ex19del L718Q | 0.632367034 | 1.546332046 | 4.812319538 | 0.716197955 |
| PACC | L858R G724S | 0.007924054 | 0.044225869 | 0.000541931 | 0.000495946 |
| PACC | Ex19del T854I | 11.68423575 | 28.95752896 | 1.509464228 | 0.254784514 |

TABLE 5.2
Response of Cells Comprising PACC EGFR Mutations to Second-Generation TKIs

| Mutation from Wildtype | | Afatinib | Dacomitinib | Neratinib | Tarlox-TKI |
|---|---|---|---|---|---|
| PACC | E709_T710del insD | 0.035976636 | 0.083906317 | 0.605730148 | 0.782034105 |
| PACC | E709K G719S | 0.00335824 | 0.001357757 | 0.048180827 | 0.065172554 |
| PACC | E709A G719S | 0.002694591 | 0.001613827 | 0.008666105 | 0.114271214 |
| PACC | E709A | 0.025417807 | 0.040239579 | 0.240111034 | 0.480288266 |
| PACC | E709K | 0.046811198 | 0.271057155 | 0.292158223 | 0.034665043 |
| PACC | L718Q | 0.381267102 | 0.382842716 | 1.430950729 | 0.069894438 |
| PACC | L718V | 0.112683646 | 0.110340567 | 1.771190641 | 1.421234267 |
| PACC | G719S | 0.022081564 | 0.002177958 | 0.012292507 | 0.002633577 |
| PACC | G719A | 0.024283342 | 0.001888241 | 0.002028247 | 0.01637434 |
| PACC | G719A L861Q | 0.041015228 | 0.002249099 | 0.0050779 | 0.497594397 |
| PACC | G719A/R776C | 0.004787729 | 0.002517619 | 0.001480024 | 0.032655298 |
| PACC | G724S | 0.167827794 | 0.237918994 | 0.621294736 | 8.498781973 |
| PACC | I740dupIPVAK | 0.038757104 | 0.013762355 | 0.09087935 | 0.572533496 |
| PACC | L747P | 0.154630604 | 0.297190589 | 3.692376326 | 2.084855867 |
| PACC | L747S | 0.012874132 | 0.006376773 | 4.12907703 | 0.173304913 |
| PACC | K757R | 0.070532519 | 0.04086807 | 0.20531377 | 0.301867641 |
| PACC | S768I | 42.75415702 | 17.6552428 | 10.70105086 | 0.386276898 |
| PACC | S768I/V769L | 0.138297201 | 0.208476579 | 0.195350451 | 0.712951685 |
| PACC | S768I V774M | 0.156377605 | 0.140696175 | 1.218994746 | 0.211713358 |
| PACC | V769L | 0.189644285 | 0.140169746 | 0.150614653 | 0.302436054 |
| PACC | V774M | 0.034369606 | 0.045128921 | 1.889907802 | 0.250730816 |
| PACC | R776H | 0.0031004 | 0.001367372 | 0.016128185 | 0.163926106 |
| PACC | R776C | 0.037813092 | 0.121545982 | 0.250966591 | 0.020091352 |
| PACC | L858R/L718V | 0.002735424 | 0.0015289 | 0.022231585 | 0.357815672 |
| PACC | L858R L718Q | 0.082014313 | 0.051004512 | 0.635620105 | 0.365529842 |
| PACC | Ex19del G724S | 0.431593349 | 0.170691878 | 0.460493754 | 2.658140479 |
| PACC | L858R/L792H | 0.016633456 | 0.002837696 | 0.046269951 | 0.346000812 |
| PACC | Ex19del/L792H | 0.245755571 | 0.243553825 | 6.744326361 | 1.040803898 |
| PACC | Ex19del G796S | 0.364659545 | 0.237134723 | 0.738103946 | 25.14616322 |
| PACC | L858R/C797S | 0.149579036 | 0.045391056 | 2.32130465 | 0.743422655 |
| PACC | Ex19del/C797S | 0.028374037 | 0.017616045 | 15.65777486 | 0.227243199 |
| PACC | Ex19del L718V | 0.001613029 | 0.006099592 | 1.296321999 | 0.12135607 |
| PACC | Ex19del L718Q | 0.038154073 | 0.03226257 | 0.040963617 | 0.096508323 |
| PACC | L858R G724S | 0.001045043 | 0.000571175 | 0.000531625 | 0.026280958 |
| PACC | Ex19del T854I | 0.152652073 | 0.412714869 | 0.358282938 | 0.701887942 |

TABLE 5.3
Response of Cells Comprising PACC EGFR Mutations to Third-Generation
TKIs

| Mutation from | | | | | | | |
|---|---|---|---|---|---|---|---|
| Wildtype | | Osimertinib | Nazartinib | Olmutinib | Rocelitinib | Naquotinib | Lazertinib |
| PACC | E709_T710del | 0.639547839 | 0.158429306 | 0.229885823 | 0.275756061 | 0.49700495 | 0.566323504 |
| | insD | | | | | | |
| PACC | E709K G719S | 1.073928787 | 0.419514139 | 0.356852302 | 0.370737316 | 0.533910891 | 0.265245158 |
| PACC | E709A G719S | 0.480571933 | 0.092365039 | 0.144269498 | 0.155611097 | 0.357425743 | 0.068782062 |
| PACC | E709A | 0.486049858 | 0.077838046 | 0.015159282 | 0.119345164 | 0.074299505 | 0.275868086 |
| PACC | E709K | 0.208718258 | 0.030899743 | 0.055620652 | 0.1051987 | 0.266311881 | 0.26117606 |
| PACC | L718Q | 10.66802841 | 1.244730077 | 3.977231117 | 0.88227943 | 5.447524752 | 5.442331181 |
| PACC | L718V | 1.195626944 | 1.156838046 | 0.138976699 | 0.751512122 | 0.542623762 | 0.618391206 |
| PACC | G719S | 0.906687619 | 0.081651157 | 0.213965803 | 0.112711825 | 0.272673267 | 0.155452801 |
| PACC | G719A | 0.73199422 | 0.067754346 | 0.140996445 | 0.109075029 | 0.134443106 | 0.361996161 |
| PACC | G719A L861Q | 0.494508254 | 0.063440987 | 0.073877021 | 0.049543463 | 0.031362624 | 1.406220555 |
| PACC | G719A/R776C | 0.441576528 | 0.057722365 | 0.180486668 | 0.172756811 | 0.289851485 | 0.522439365 |
| PACC | G724S | 1.164871663 | 0.868964093 | 0.18270034 | 0.979730067 | 1.173762376 | 26.17344268 |
| PACC | I740dupIPVAK | 0.891996658 | 0.135236504 | 0.171462335 | 0.324843789 | 0.5025 | 0.288256849 |
| PACC | L747P | 0.653103384 | 0.222082262 | 0.392397057 | 0.472556861 | 3.506683168 | 2.919909265 |
| PACC | L747S | 0.180423379 | 0.063023136 | 0.06290736 | 0.185728568 | 0.891089109 | 1.007328564 |
| PACC | K757R | 0.835615802 | 0.348560411 | 0.143870078 | 0.700374906 | 1.657920792 | 0.348961787 |
| PACC | S768I | 0.365338865 | 0.527326478 | 0.007216471 | 0.851762059 | 1.089921931 | 1.259815041 |
| PACC | S768I/V769L | 0.633257509 | 0.225321337 | 0.358210446 | 0.427943014 | 1.734405941 | 0.666026871 |
| PACC | S768I V774M | 0.231600204 | 0.09562982 | 0.110948371 | 0.120294926 | 0.491707921 | 0.767754319 |
| PACC | V769L | 1.150828652 | 0.1771491 | 0.312140075 | 0.780254936 | 1.624009901 | 1.618042226 |
| PACC | V774M | 0.029826842 | 0.117943445 | 0.069877834 | 0.082054486 | 0.286188119 | 0.96353167 |
| PACC | R776H | 0.124112158 | 0.036010283 | 0.114576745 | 0.186178455 | 0.509529703 | 0.607747339 |
| PACC | R776C | 1.637342742 | 0.284601542 | 0.513065477 | 1.394651337 | 4.26980198 | 1.504100506 |
| PACC | L858R/L718V | 4.424283924 | 1.029614396 | 0.862854099 | 0.955411147 | 3.357623762 | 2.619106613 |
| PACC | L858R L718Q | 12.61501323 | 0.62344473 | 2.324156985 | 2.970032492 | 5.547029703 | 3.063688711 |
| PACC | Ex19del G724S | 2.274964022 | 1.067532134 | 0.268033687 | 0.918270432 | 0.886633663 | 1.781922876 |
| PACC | L858R/L792H | 0.55094926 | 0.314457548 | 0.140501238 | 0.447412372 | 0.262351522 | 0.141633223 |
| PACC | Ex19del/ | 1.534260434 | 0.514627172 | 0.019233714 | 0.927893302 | 0.029287111 | 0.001792183 |
| | L792H | | | | | | |
| PACC | Ex19del G796S | 2.066524302 | 0.660411311 | 0.823441297 | 0.567733067 | 1.13740099 | 1.423486303 |
| PACC | L858R/C797S | 16.49644399 | 3.460411311 | 2.241937019 | 0.87488158 | 5.265841584 | 28.05444076 |
| PACC | Ex19del/ | 10.04317255 | 3.067093059 | 2.140074898 | 1.086228193 | 11.44554703 | 11.81818182 |
| | C797S | | | | | | |
| PACC | Ex19del L718V | 0.380281788 | 0.046323907 | 0.086198862 | 0.096975756 | 0.103514851 | 0.049436399 |
| PACC | Ex19del L718Q | 0.341140151 | 0.038010283 | 0.033887021 | 0.073684079 | 0.055856436 | 0.082376549 |
| PACC | L858R G724S | 0.054547143 | 0.009244216 | 0.020521288 | 0.092061985 | 0.57990099 | 0.000464736 |
| PACC | Ex19del T854I | 0.931479504 | 1.134755784 | 1.309899804 | 1.167508123 | 1.060460396 | 1.439888327 |

TABLE 5.4
Response of Cells Comprising PACC EGFR
Mutations to Ex20ins-Specific TKIs

| | | | | TAK-788 |
|---|---|---|---|---|
| Mutation from Wildtype | | TAS 6417 | AZ5104 | (mobocertinib) |
| PACC | E709_T710del | 0.333476578 | 0.030626037 | 0.019795571 |
| | insD | | | |
| PACC | E709K G719S | 0.007578411 | 0.797299904 | 2.045553663 |
| PACC | E709A G719S | 0.034716904 | 0.796991321 | 1.297785349 |
| PACC | E709A | 0.305437882 | 0.21903568 | 1.583191369 |
| PACC | E709K | 0.251588595 | 0.206981678 | 10.67575241 |
| PACC | L718Q | 0.022393075 | 11.31918997 | 16.38160136 |
| PACC | L718V | 0.023781466 | 2.62246866 | 39.29358319 |
| PACC | G719S | 0.016745418 | 0.3756027 | 2.056218058 |
| PACC | G719A | 0.01801222 | 0.189566056 | 1.675752413 |
| PACC | G719A L861Q | 0.02386558 | 0.804387657 | 1.066098807 |
| PACC | G719A/R776C | 3.054989817 | 0.213577628 | 0.814400909 |
| PACC | G724S | 3.054989817 | 36.15236258 | 108.5973878 |
| PACC | I740dupIPVAK | 0.355478615 | 2.233847637 | 7.184554231 |
| PACC | L747P | 0.194949084 | 3.589874638 | 5.990914253 |
| PACC | L747S | 0.064818737 | 4.200675024 | 2.299375355 |
| PACC | K757R | 0.460753564 | 0.189363549 | 1.562521295 |
| PACC | S768I | 0.084602851 | 1.361330762 | 0.776581488 |
| PACC | S768I/V769L | 0.004702037 | 0.59903568 | 0.049937535 |
| PACC | S768I V774M | 0.399566191 | 0.580906461 | 1.226689381 |
| PACC | V769L | 0.15898167 | 1.366730955 | 3.453719478 |
| PACC | V774M | 0.328167006 | 0.563452266 | 1.453378762 |
| PACC | R776H | 0.09 | 3.408871745 | 2.852810903 |
| PACC | R776C | 0.107240326 | 0.101890068 | 0.042350937 |
| PACC | L858R/L718V | 0.015087576 | 1.63972999 | 3.491005111 |
| PACC | L858R L718Q | 0.015696538 | 1.63490839 | 2.996479273 |
| PACC | Ex19del G724S | 0.280916497 | 3.177820636 | 3.681771721 |
| PACC | L858R/L792H | 0.004610998 | 4.407907425 | 0.045703578 |
| PACC | Ex19del/ | 0.008268839 | 1.777627772 | 6.547416241 |
| | L792H | | | |
| PACC | Ex19del G796S | 0.065535642 | 0.880665381 | 32.48154458 |
| PACC | L858R/C797S | 3.054989817 | 119.1899711 | 57.42191936 |
| PACC | Ex19del/C797S | 1.093482688 | 49.96142719 | 43.27086882 |
| PACC | Ex19del | 0.000936354 | 1.339054966 | 1.296422487 |
| | L718V | | | |
| PACC | Ex19del | 2.333197556 | 1.486557377 | 1.285633163 |
| | L718Q | | | |
| PACC | L858R G724S | 0.016308758 | 0.001759499 | 0.01089381 |
| PACC | Ex19del | 0.015197556 | 1.698746384 | 4.793626349 |
| | T854I | | | |

TABLE 5.5
List of Example PACC EGFR Mutations
Mutation from Wildtype

| | |
|---|---|
| PACC | A750_I759del insPN |
| PACC | E709_T710del insD |
| PACC | E709A |
| PACC | E709A G719A |
| PACC | E709A G719S |
| PACC | E709K |
| PACC | E709K G719S |
| PACC | E736K |
| PACC | E746_A750del A647T |
| PACC | E746_A750del R675W |
| PACC | E746_T751del insV S768C |
| PACC | Ex19del C797S |
| PACC | Ex19del G796S |
| PACC | Ex19del L792H |
| PACC | Ex19del T854I |
| PACC | G719A |
| PACC | G719A D761Y |
| PACC | G719A L861Q |
| PACC | G719A R776C |
| PACC | G719A S768I |
| PACC | G719C S768I |
| PACC | G719S |
| PACC | G719S L861Q |
| PACC | G719S S768I |
| PACC | G724S |
| PACC | G724S Ex19del |
| PACC | G724S L858R |
| PACC | G779F |
| PACC | I740dupIPVAK |
| PACC | K757M L858R |
| PACC | K757R |
| PACC | L718Q |
| PACC | Ex19del |
| PACC | L718Q L858R |
| PACC | L718V |
| PACC | L718V L858R |
| PACC | L747_S752del A755D |
| PACC | L747P |
| PACC | L747S |
| PACC | L747S L858R |
| PACC | L747S V774M |
| PACC | L858R C797S |
| PACC | L858R L792H |
| PACC | L858R T854S |
| PACC | N771G |
| PACC | R776C |
| PACC | R776H |
| PACC | E709_T710del insD S22R |
| PACC | S752_I759del V769M |
| PACC | S768I |
| PACC | S768I L858R |
| PACC | S768I L861Q |
| PACC | S768I V769L |
| PACC | S768I V774M |
| PACC | T751_I759 delinsN |
| PACC | V769L |
| PACC | V769M |
| PACC | V774M |

Thus, in some embodiments, disclosed are methods for treating a subject for lung cancer, the method comprising administering an effective amount of one or more second-generation EGFR inhibitors (e.g., afatinib, dacomitinib, neratinib, tarlox-TKI, tarloxotinib, or a combination thereof) to a subject determined, from analysis of tumor DNA from the subject, to have an EGFR mutation, wherein the EGFR mutation a PACC mutation. In some aspects, the PACC mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M. Also disclosed are methods for treating a subject for lung cancer, the method comprising: (a) detecting an EGFR mutation in tumor DNA from the subject, wherein the EGFR mutation is a PACC mutation; and (b) administering an effective amount of one or more second-generation EGFR inhibitors (e.g., afatinib, dacomitinib, neratinib, tarlox-TKI, tarloxotinib, or a combination thereof) to the subject. In some aspects, the PACC mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M. Further disclosed are methods comprising administering a tyrosine kinase inhibitor to a subject determined, from analysis of tumor DNA from the subject, to have a P-loop αC-helix compressing EGFR mutation, wherein the tyrosine kinase inhibitor is not an EGFR inhibitor.

In some embodiments, the method comprises administering afatinib to the subject. In some embodiments, the method comprises administering dacomitinib to the subject. In some embodiments, the method comprises administering neratinib to the subject. In some embodiments, the method comprises administering tarlox-TKI to the subject. In some embodiments, the method comprises administering tarloxotinib to the subject.

In some embodiments, the EGFR mutation is A750_I759del insPN. In some embodiments, the EGFR mutation is E709_T710del insD. In some embodiments, the EGFR mutation is E709A. In some embodiments, the EGFR mutation is E709A G719A. In some embodiments, the EGFR mutation is E709A G719S. In some embodiments, the EGFR mutation is E709K. In some embodiments, the EGFR mutation is E709K G719S. In some embodiments, the EGFR mutation is E736K. In some embodiments, the EGFR mutation is E746_A750del A647T. In some embodiments, the EGFR mutation is E746_A750del R675W. In some embodiments, the EGFR mutation is E746_T751del insV S768C. In some embodiments, the EGFR mutation is Ex19del C797S. In some embodiments, the EGFR mutation is Ex19del G796S. In some embodiments, the EGFR mutation is Ex19del L792H. In some embodiments, the EGFR mutation is Ex19del T854I. In some embodiments, the EGFR mutation is G719A. In some embodiments, the EGFR mutation is G719A D761Y. In some embodiments, the EGFR mutation is G719A L861Q. In some embodiments, the EGFR mutation is G719A R776C. In some embodiments, the EGFR mutation is G719A S768I. In some embodiments, the EGFR mutation is G719C S768I. In some embodiments, the EGFR mutation is G719S. In some embodiments, the EGFR mutation is G719S L861Q. In some embodiments, the EGFR mutation is G719S S768I. In some embodiments, the EGFR mutation is G724S. In some embodiments, the EGFR mutation is G724S Ex19del. In some embodiments, the EGFR mutation is G724S L858R. In some embodiments, the EGFR mutation is G779F. In some embodiments, the EGFR mutation is I740dupIPVAK. In some embodiments, the EGFR mutation is K757M L858R. In some embodiments, the EGFR mutation is K757R. In some embodiments, the EGFR mutation is L718Q. In some embodiments, the EGFR mutation is Ex19del. In some embodiments, the EGFR mutation is L718Q L858R. In some embodiments, the EGFR mutation is L718V. In some embodiments, the EGFR mutation is L718V L858R. In some embodiments, the EGFR mutation is L747_S752del A755D. In some embodiments, the EGFR mutation is L747P. In some embodiments, the EGFR mutation is L747S. In some embodiments, the EGFR mutation is L747S L858R. In some embodiments, the EGFR mutation is L747S V774M. In some embodiments, the EGFR mutation is L858R C797S. In some embodiments, the EGFR mutation is L858R L792H. In some embodiments, the EGFR mutation is L858R T854S. In some embodiments, the EGFR mutation is N771G. In some embodiments, the EGFR mutation is R776C. In some embodiments, the EGFR mutation is R776H. In some embodiments, the EGFR mutation is E709_T710del insD S22R. In some embodiments, the EGFR mutation is S752_I759del V769M. In some embodiments, the EGFR mutation is S768I. In some embodiments, the EGFR mutation is S768I L858R. In some embodiments, the EGFR mutation is S768I L861Q. In some embodiments, the EGFR mutation is S768I V769L. In some embodiments, the EGFR mutation is S768I V774M. In some embodiments, the EGFR mutation is T751_I759 delinsN. In some embodiments, the EGFR mutation is V769L. In some embodiments, the EGFR mutation is V769M. In some embodiments, the EGFR mutation is V774M.

In some embodiments, the subject was previously treated with a cancer therapy. In some embodiments, the cancer therapy comprised erlotinib, gefitinib, AZD3759, or sapatinib. In some embodiments, the cancer therapy comprised osimertinib, nazartinib, olmutinib, rocelitinib, naquotinib, lazertinib. In some embodiments, the cancer therapy comprised chemotherapy. In some embodiments, the subject was determined to be resistant to the cancer therapy.

In some embodiments, the disclosed methods comprise identifying one or more subjects as being candidates for treatment with one or more kinase inhibitors from one or more kinase inhibitor classes based on the presence or absence of one or more mutations in the EGFR gene of a tumor of the subject. For example, in some embodiments, disclosed is a method comprising identifying a subject having cancer (e.g., lung cancer) as being a candidate for treatment with one or more kinase inhibitors from one or more kinase inhibitor classes by determining that the efficacy of the one or more kinase inhibitors from one or more kinase inhibitor classes is or would be optimal. In some cases, one or more kinase inhibitors from one or more kinase inhibitor classes is or would be optimal when the subject is determined to have one or more mutations in the EGFR gene in a tumor of the subject that confer increased sensitivity (or decreased resistance) to the one or more kinase inhibitors. In some cases, one or more kinase inhibitors from one or more kinase inhibitor classes is or would be suboptimal when the subject is determined to have one or more mutations in the EGFR gene in a tumor of the subject that confer decreased sensitivity (or increased resistance) to the one or more kinase inhibitors. In some embodiments, the disclosed methods comprise determining an optimal cancer treatment for a subject for whom a current or former cancer treatment is or was suboptimal. In some embodiments, a subject is given multiple types of cancer therapy, for example multiple kinase inhibitor therapies.

In particular embodiments, the disclosure concerns methods of predicting sensitivity or resistance to one or more kinase inhibitors from one or more kinase inhibitor classes in a subject having cancer based on analyzing one or more of the following biomarkers in a tumor of the subject: (1) classical-like EGFR mutations; (2) exon 20 near-loop insertion (ex20ins-NL) EGFR mutations; (3) exon 20 far-loop insertion (ex20ins-FL) EGFR mutations, (4) T790M-like-3S EGFR mutations; (5) T790M-like-3R EGFR mutations; or (6) PACC EGFR mutations.

In some embodiments, the disclosure concerns methods of predicting a therapy outcome for a subject having cancer (e.g., lung cancer) and in need of treatment with one or more kinase inhibitors from one or more kinase inhibitor classes, including the likelihood of sensitivity or resistance to the one or more kinase inhibitors from one or more kinase inhibitor classes. Such analysis of (1), (2), (3), (4), (5), or (6) of the above results in a determination of whether or how best to treat the cancer or which of the one or more kinase inhibitors from one or more kinase inhibitor classes to administer to treat the cancer.

Thus, in some embodiments, the likelihood of sensitivity or resistance to a given kinase inhibitor is determined based on analysis of tumor DNA of a subject having cancer (e.g., lung cancer) for one or more mutations in the EGFR gene of the subject. In such cases, as a result of the tumor DNA analysis, targeted therapeutic strategies to treat the cancer are administered to the subject. For example, the subject may be given a therapeutically effective amount of one or more kinase inhibitors from one or more kinase inhibitor classes.

When the analysis of tumor DNA of a subject having cancer (e.g., lung cancer) for one or more mutations in the EGFR gene indicates that the subject has one or more classical-like EGFR mutations, the subject may have an increased likelihood of sensitivity (or decreased likelihood of resistance) to one or more first-generation EGFR TKIs, second-generation EGFR TKIs, third-generation EGFR TKIs, or EGFR TKIs specific to mutations associated with EGFR exon 20, and the subject may, in some cases, then be provided a therapeutically effective amount of one or more first-generation EGFR TKIs, second-generation EGFR TKIs, third-generation EGFR TKIs, or EGFR TKIs specific to mutations associated with EGFR exon 20. When the analysis of tumor DNA of a subject having cancer (e.g., lung cancer) for one or more mutations in the EGFR gene indicates that the subject does not have one or more classical-like EGFR mutations, the subject may have a decreased likelihood of sensitivity (or increased likelihood of resistance) to one or more first-generation EGFR TKIs, second-generation EGFR TKIs, third-generation EGFR TKIs, or EGFR TKIs specific to mutations associated with EGFR exon 20, and the subject may, in some cases, then be provided a therapeutically effective amount of one or more alternative kinase inhibitors.

When the analysis of tumor DNA of a subject having cancer (e.g., lung cancer) for one or more mutations in the EGFR gene indicates that the subject has one or more ex20ins-NL EGFR mutations, the subject may have an increased likelihood of sensitivity (or decreased likelihood of resistance) to one or more second-generation EGFR TKIs or EGFR TKIs specific to mutations associated with EGFR exon 20, and the subject may, in some cases, then be provided a therapeutically effective amount of one or more second-generation EGFR TKIs or EGFR TKIs specific to mutations associated with EGFR exon 20. When the analysis of tumor DNA of a subject having cancer (e.g., lung cancer) for one or more mutations in the EGFR gene indicates that the subject does not have one or more ex20ins-NL EGFR mutations, the subject may have a decreased likelihood of sensitivity (or increased likelihood of resistance) to one or more second-generation EGFR TKIs or EGFR TKIs specific to mutations associated with EGFR exon 20, and the subject may, in some cases, then be provided a therapeutically effective amount of one or more alternative kinase inhibitors.

When the analysis of tumor DNA of a subject having cancer (e.g., lung cancer) for one or more mutations in the EGFR gene indicates that the subject has one or more T790M-like-3S EGFR mutations, the subject may have an increased likelihood of sensitivity (or decreased likelihood of resistance) to one or more third-generation EGFR TKIs, EGFR TKIs specific to mutations associated with EGFR exon 20, ALK inhibitors, or PKC inhibitors, and the subject may, in some cases, then be provided a therapeutically effective amount of one or more third-generation EGFR TKIs, EGFR TKIs specific to mutations associated with EGFR exon 20, ALK inhibitors, or PKC inhibitors. When the analysis of tumor DNA of a subject having cancer (e.g., lung cancer) for one or more mutations in the EGFR gene indicates that the subject does not have one or more T790M-like-3S EGFR mutations, the subject may have a decreased likelihood of sensitivity (or increased likelihood of resistance) to one or more third-generation EGFR TKIs, EGFR TKIs specific to mutations associated with EGFR exon 20, ALK inhibitors, or PKC inhibitors, and the subject may, in some cases, then be provided a therapeutically effective amount of one or more alternative kinase inhibitors.

When the analysis of tumor DNA of a subject having cancer (e.g., lung cancer) for one or more mutations in the EGFR gene indicates that the subject has one or more T790M-like-3R EGFR mutations, the subject may have an increased likelihood of sensitivity (or decreased likelihood of resistance) to one or more ALK inhibitors or PKC inhibitors, and the subject may, in some cases, then be provided a therapeutically effective amount of one or more ALK inhibitors or PKC inhibitors. When the analysis of tumor DNA of a subject having cancer (e.g., lung cancer) for one or more mutations in the EGFR gene indicates that the subject does not have one or more T790M-like-3R EGFR mutations, the subject may have a decreased likelihood of sensitivity (or increased likelihood of resistance) to one or more ALK inhibitors or PKC inhibitors, and the subject may, in some cases, then be provided a therapeutically effective amount of one or more alternative kinase inhibitors.

When the analysis of tumor DNA of a subject having cancer (e.g., lung cancer) for one or more mutations in the EGFR gene indicates that the subject has one or more PACC EGFR mutations, the subject may have an increased likelihood of sensitivity (or decreased likelihood of resistance) to one or more second-generation EGFR TKIs, and the subject may, in some cases, then be provided a therapeutically effective amount of one or more second-generation EGFR TKIs. When the analysis of tumor DNA of a subject having cancer (e.g., lung cancer) for one or more mutations in the EGFR gene indicates that the subject does not have one or more PACC EGFR mutations, the subject may have a decreased likelihood of sensitivity (or increased likelihood of resistance) to one or more second-generation EGFR TKIs, and the subject may, in some cases, then be provided a therapeutically effective amount of one or more alternative kinase inhibitors.

### III. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from lung tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. In some aspects, a sample is a cancerous or non-cancerous lung tissue sample. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple lung tissue samples may be obtained for diagnosis by the methods described herein. In other cases, multiple samples, such as one or more samples from one tissue type (for example lung) and one or more samples from another specimen (for example serum or blood) may be obtained for diagnosis by the methods. In some cases, multiple samples such as one or more samples from one tissue type (e.g. lung) and one or more samples from another specimen (e.g. serum or blood) may be obtained at the same or different times. Samples may be obtained at different times are stored and/or analyzed by different methods. For example, a sample may be obtained and analyzed by routine staining methods or any other cytological analysis methods, by sequencing (e.g., DNA or RNA sequencing), by microarray, or by any other genetic analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

In some cases, a biological sample is a cell-free sample (e.g., a serum sample). In such cases, a biological sample may contain cell-free nucleic acids such as DNA (e.g., cell-free tumor DNA, cell-free fetal DNA) or RNA (e.g., cell-free tumor RNA, cell-free fetal RNA). In some aspects, a cell-free biological sample contains, or is suspected of containing, DNA or RNA from lung cancer.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a lung or a suspected lung tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Assay Methods

### A. Sequencing

In some embodiments, the methods of the disclosure include a sequencing method. Exemplary sequencing methods include those described below.

### 1. Massively Parallel Signature Sequencing (MPSS)

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. The essential properties of the MPSS output were typical of later “next-generation” data types, including hundreds of thousands of short DNA sequences. In the case of MPSS, these were typically used for sequencing cDNA for measurements of gene expression levels.

### 2. Polony Sequencing

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing.

### 3. 454 Pyrosequencing

A parallelized version of pyrosequencing amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### 4. Illumina (Solexa) Sequencing

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony).

### 5. Solid Sequencing

SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing.

### 6. Ion Torrent Semiconductor Sequencing

Ion Torrent Systems Inc. developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### 8. Heliscope Single Molecule Sequencing

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer.

### 9. Single Molecule Real Time (SMRT) Sequencing

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. This approach allows reads of 20,000 nucleotides or more, with average read lengths of 5 kilobases.

### B. Additional Assay Methods

In some embodiments, methods involve amplifying and/or sequencing one or more target genomic regions using at least one pair of primers specific to the target genomic regions. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991, each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, ddPCR (droplet digital PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and J-2 microglobulin. These standards may be used to normalize expression levels so that the expression levels of different gene products can be compared directly. A person of ordinary skill in the art would know how to use an internal standard to normalize expression levels.

### V. Administration of Therapeutic Compositions

The therapy provided herein comprises administration of a combination of therapeutic agents, including at least one or more kinase inhibitors from one or more kinase inhibitor classes. In some embodiments, at least 1, 2, 3, 4, 5, or 6 classes of TKIs are administered. In some embodiments, the 1, 2, 3, 4, 5, or 6 TKI classes comprise first-generation EGFR TKIs, second-generation EGFR TKIs, third-generation EGFR TKIs, EGFR TKIs specific to mutations associated with EGFR exon 20, ALK inhibitors, or PLC inhibitors. In some embodiments, the TKI is Erlotinib, Geftinib, AZD3759, Sapatinib, Afatinib, Dacomitinib, Neratinib, Tarlox-TKI, TAS 6417, AZ5104, TAK-788 (mobocertinib), Osimertinib, Nazartinib, Olmutinib, Rocelitinib, Naquotinib, Lazertinib, AZD3463, Brigatinib, Ruboxistaurin, Midostaurin, or Sotrastaurin. In some embodiments, at least 1, 2, 3, 4, 5, or more TKIs are administered to a subject having cancer. In some embodiments, the one or more TKIs comprise two or more of Erlotinib, Geftinib, AZD3759, Sapatinib, Afatinib, Dacomitinib, Neratinib, Tarlox-TKI, TAS 6417, AZ5104, TAK-788 (mobocertinib), Osimertinib, Nazartinib, Olmutinib, Rocelitinib, Naquotinib, Lazertinib, AZD3463, Brigatinib, Ruboxistaurin, Midostaurin, or Sotrastaurin. Any one or more TKIs may be excluded from certain embodiments of the disclosure.

Embodiments of the disclosure relate to compositions and methods comprising therapeutic compositions. In some embodiments, the different therapies are administered sequentially (at different times) or concurrently (at the same time). The different therapies may be administered in one composition or in more than one composition, such as 2 compositions, 3 compositions, or 4 compositions. In some embodiments, the therapies are administered in a separate composition. In some embodiments, the therapies are in the same composition. In some embodiments of the methods disclosed herein, a single dose of the cancer therapies are administered. In some embodiments of the methods disclosed herein, multiple doses of the cancer therapies are administered. Various combinations of the agents may be employed. For example, a first-generation TKI is “A” and a second-generation TKI is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Compositions according to the present disclosure can be prepared according to standard techniques and may comprise water, buffered water, saline, glycine, dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. The preparation of compositions that contains the cancer therapies will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety, and purity standards as required by FDA Office of Biological Standards.

The compositions will be pharmaceutically acceptable or pharmacologically acceptable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in therapeutic compositions is contemplated.

The carrier may be a solvent or dispersion medium containing, for example, water, ethanol, polyol (for example, glycerol, propylene glycol, and liquid polyethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The cancer therapies or therapeutic agents of the disclosure may be administered by the same route of administration or by different routes of administration. In some embodiments, the cancer therapy is administered intraarterially, intravenously, intraperitoneally, subcutaneously, intramuscularly, intratumorally, topically, orally, transdermally, intraorbitally, by implantation, by inhalation, intrathecally, intraventricularly, or intranasally. The appropriate dosage may be determined based on the type of disease to be treated, severity and course of the disease, the clinical condition of the subject, the subject's clinical history and response to the treatment, and the discretion of the attending physician.

The treatments may include various “unit doses.” Unit dose is defined as containing a predetermined-quantity of the therapeutic composition calculated to produce the desired responses discussed above in association with its administration, i.e., the appropriate route and regimen. The quantity to be administered, and the particular route and formulation, is within the skill of determination of those in the clinical arts and depends on the result and/or protection desired. A unit dose need not be administered as a single injection but may comprise continuous infusion over a set period of time. In some embodiments, a unit dose comprises a single administrable dose.

Typically, compositions are administered in a manner compatible with the dosage formulation, and in such amount as will be therapeutically or prophylactically effective for the subject being treated. Precise amounts of the therapeutic composition also depend on the judgment of the practitioner and are peculiar to each individual. Suitable regimes for initial administration and boosters are also variable, but are typified by an initial administration followed by subsequent administrations. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for one or more biomarkers

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### 1. Structure-Function Based Groups Predict EGFR TKI Sensitivity Better than Exon-Based Groups

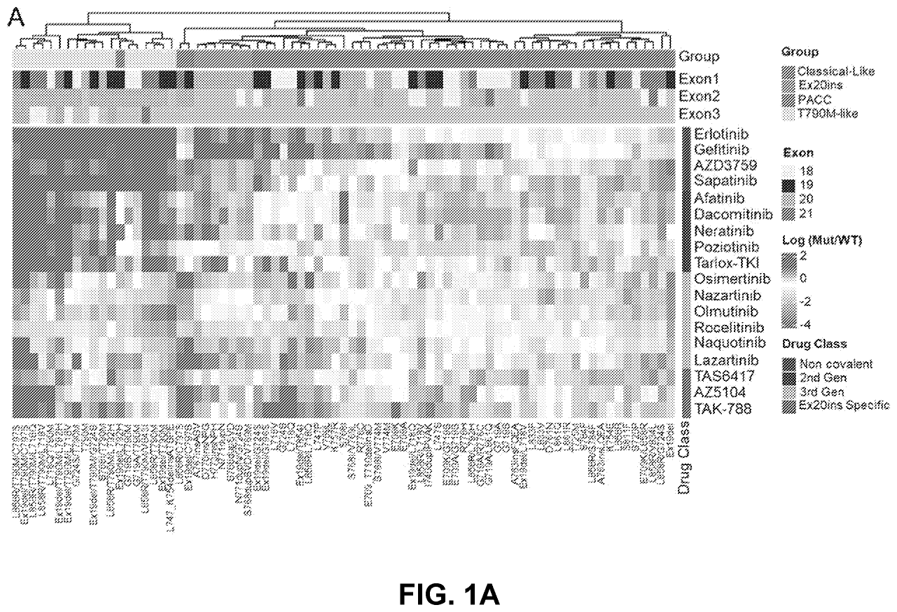

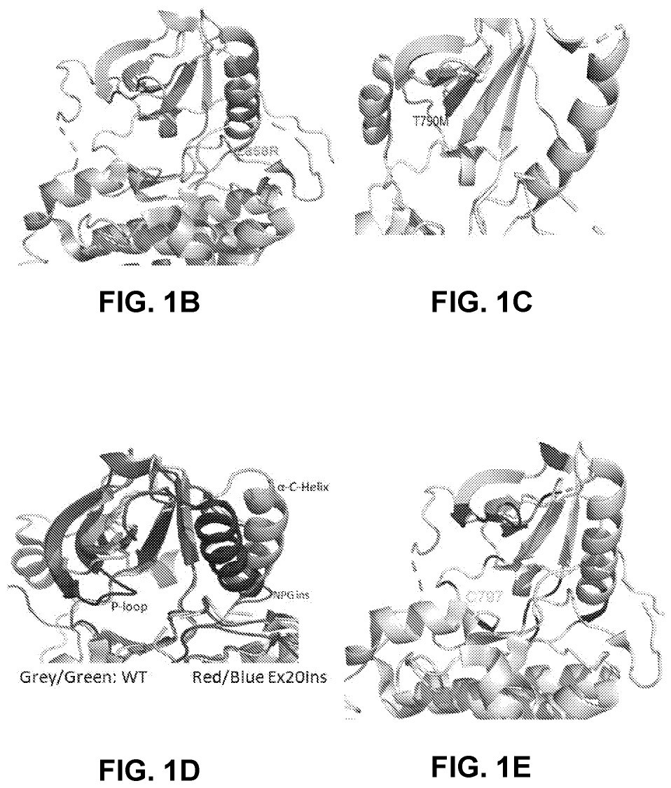

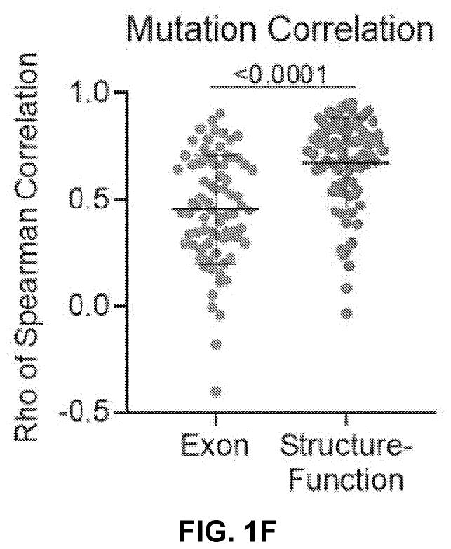

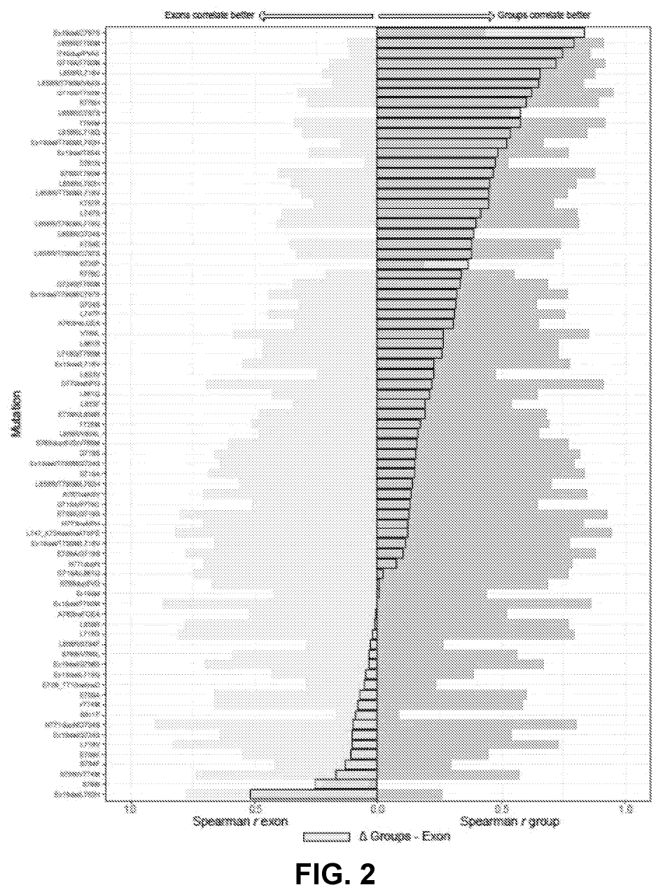

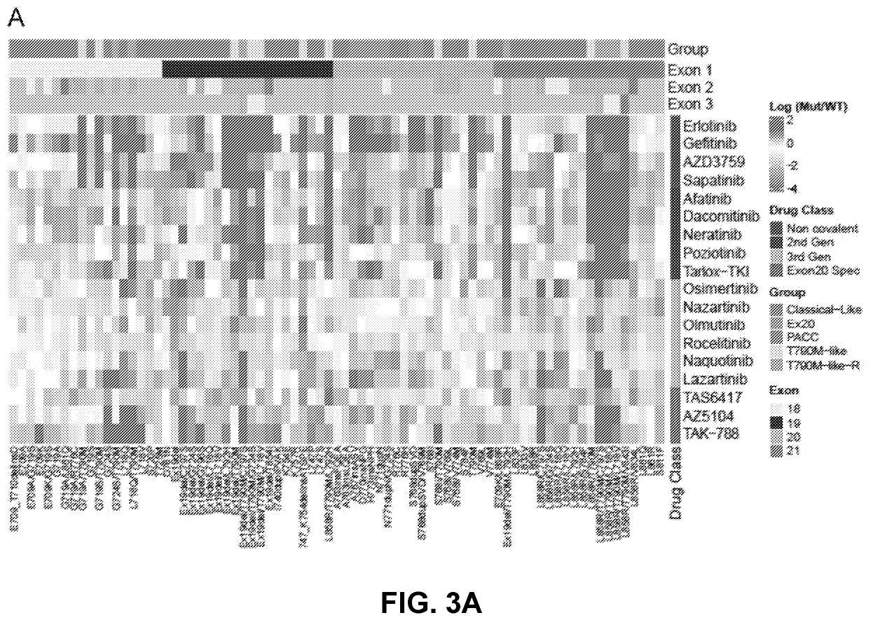

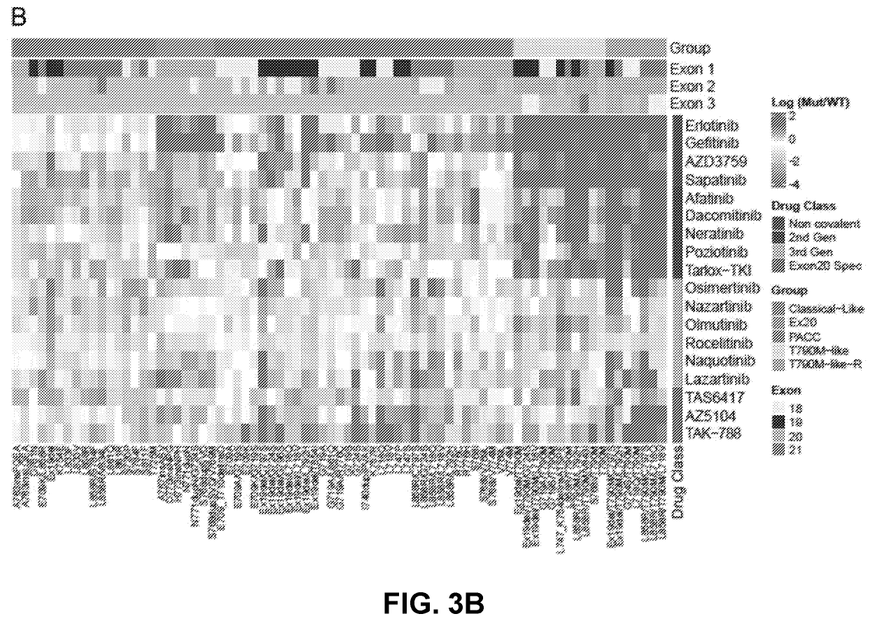

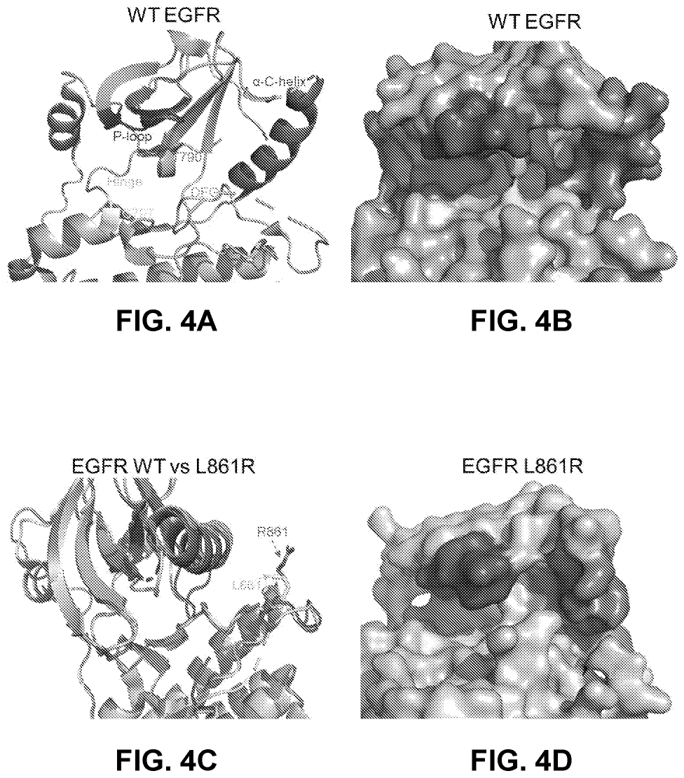

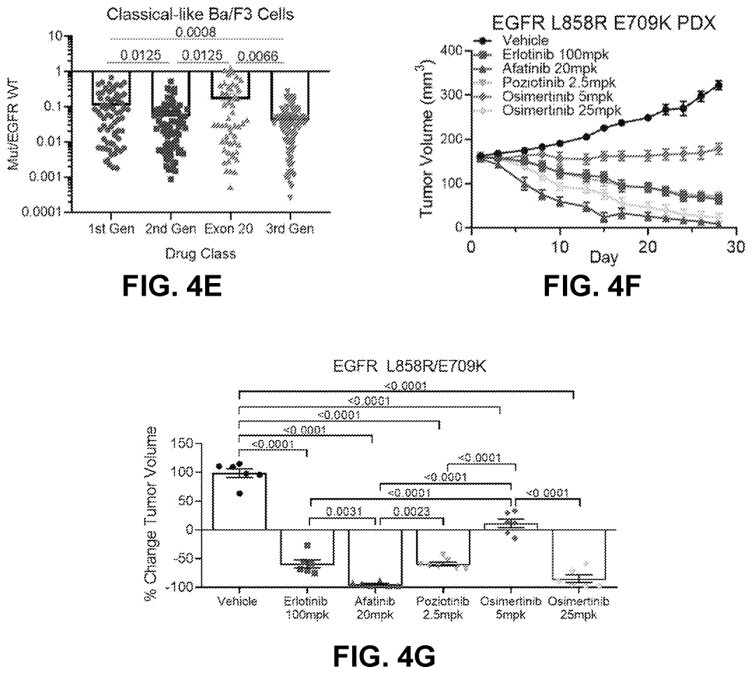

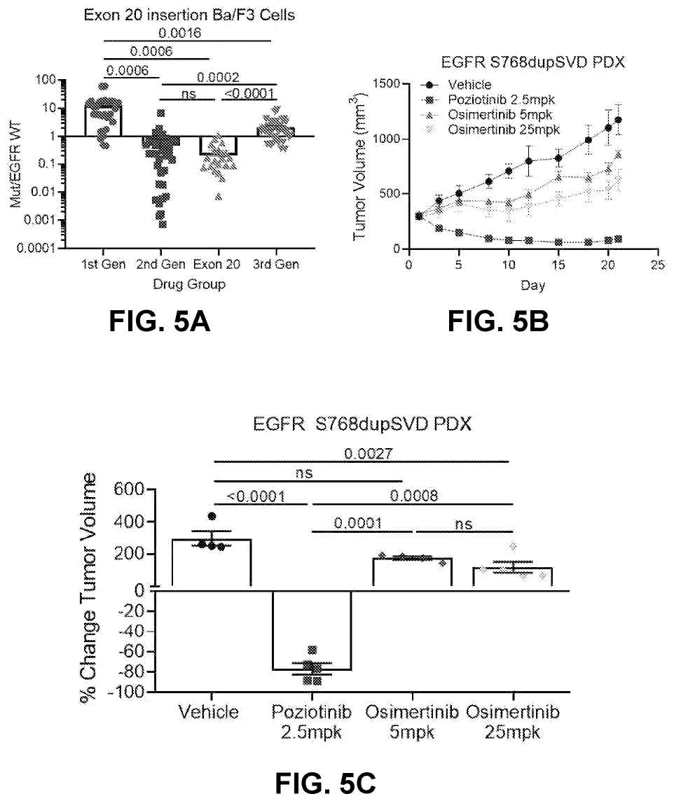

To determine the effect of EGFR mutations on TKI sensitivity, a panel of 76 cell lines expressing EGFR mutations spanning exons 18-21 was generated and screened against 18 known EGFR inhibitors representing 1^{st} (non-covalent), 2^{nd} (covalent), and 3^{rd} (covalent, T790M targeting) generation TKIs, and compounds under investigation for Ex20ins. Through hierarchical clustering of in vitro selectivity over WT EGFR and mutational mapping of EGFR mutations, four distinct subgroups of EGFR mutations were observed: classical-like mutations that were distant from the ATP binding pocket (FIG. 1A, FIG. 1B) T790M-like mutations in the hydrophobic core (FIG. 1A, FIG. 1C), Ex20ins at the c-terminal of the α-c-helix (FIG. 1A, FIG. 1D), and a fourth group on the interior surface of the ATP binding pocket and c-terminal of the α-c-helix, which were predicted to be P-loop and α-C-helix compressing (PACC) mutations (FIG. 1A, FIG. 1E). For the various mutations, structure-function based groups were more predictive of drug sensitivity than exon based groups as determined by spearmen correlations (p<0.0001, FIG. 1F, FIG. 2). Supervised clustering by structure-function based group maintained distinct groupings within the heatmap. However, supervised clustering by exon location appeared to disorder drug sensitivity patterns on the heatmap (FIG. 3). Classical-like, atypical EGFR mutations were predicted to have little impact on the overall structure of EGFR compared to WT EGFR (FIGS. 4A-4D), and were sensitive and selective for all classes of EGFR TKIs, particularly third-generation TKIs in vitro (FIG. 4E) and in vivo (FIG. 4F, FIG. 4G). Ex20ins mutations were resistant to first and third-generation TKIs and were sensitive only to select second-generation TKIs (e.g., tarlox-TKI) and ex20ins specific TKIs in vitro (FIG. 5A) and in vivo (FIG. 5C, FIG. 5D). These findings demonstrate that structure-function based groups can predict drug class sensitivity for a given a mutation and can predict which groups of mutations are most sensitive to a given inhibitor more effectively than traditional exon based grouping.

### 2. EGFR TKI Resistant T790M-Like Mutations can be Inhibited by ALK and PKC Inhibitors

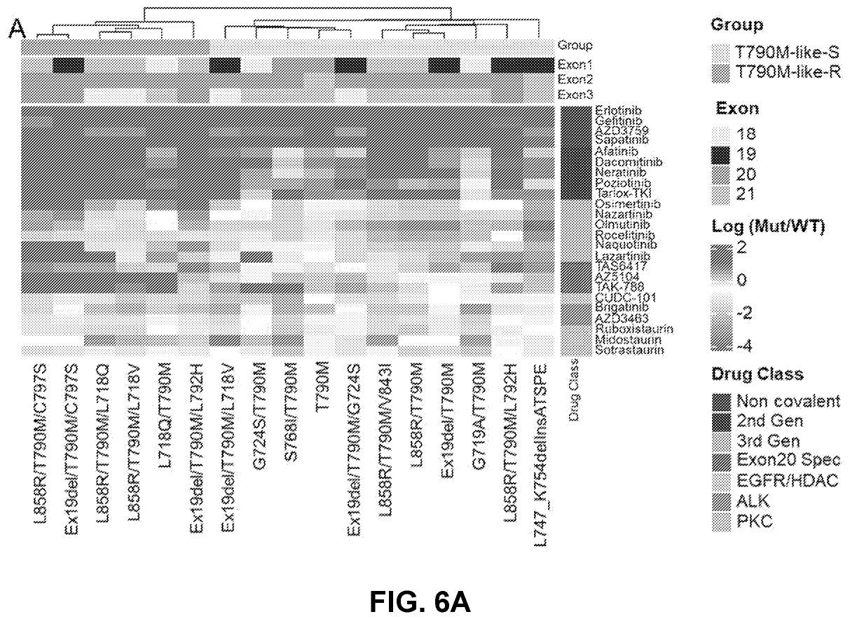

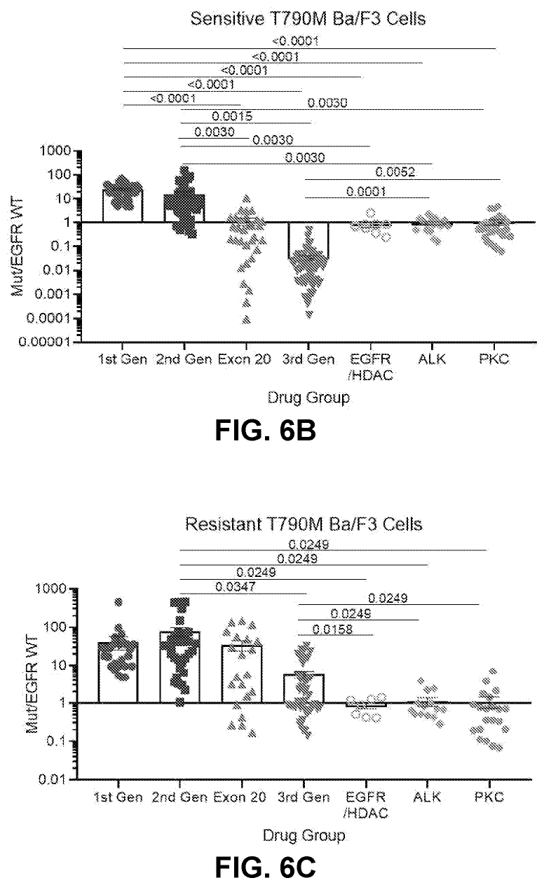

While all T790M-like mutants had at least one mutation in the hydrophobic core, there were two distinct subgroups of T790M-like mutants, third-generation TKI sensitive (T790M-like-3S) and third-generation TKI resistant (T790M-like-3R, FIG. 6A). T790M-like-3S mutants had high selectivity for third-generation TKIs and some exon 20 specific inhibitors and moderate selectivity for ALK and PKC inhibitors (FIG. 6B). T790M-like-3R mutants were complex mutations comprised of T790M and a known drug resistance mutation (i.e. C797S^{37,38}, L718X^{18,24}, or L792H^{23,24}), and were resistant to classical EGFR TKIs but retained selectivity for select ALK and PKC inhibitors (FIG. 6C). Taken together these data demonstrate that T790M-like mutants contained at least one mutation in the hydrophobic cleft, which is known to convey resistance to first and second-generation EGFR TKIs, but the addition of a known resistance mutations caused reduced sensitivity to classical EGFR TKIs that could be overcome by drug repurposing with ALK or PKC inhibitors.

### 3. PACC Mutations are Most Sensitive to Second-Generation EGFR TKIs

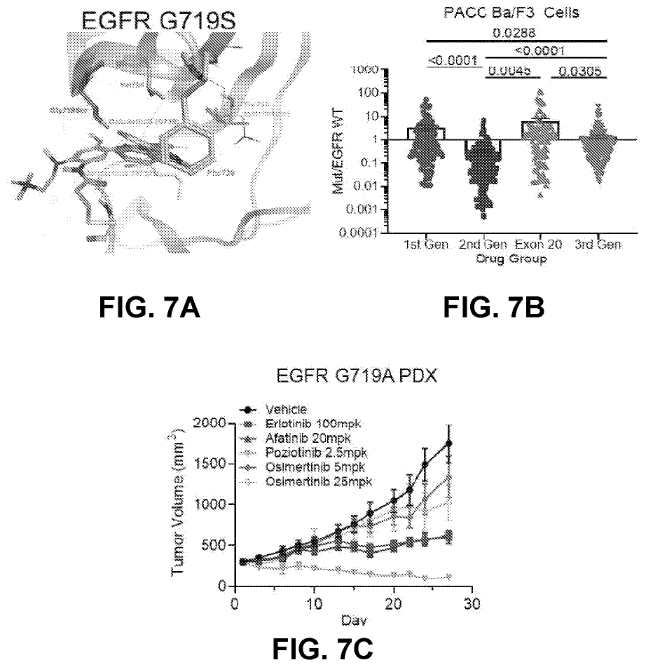

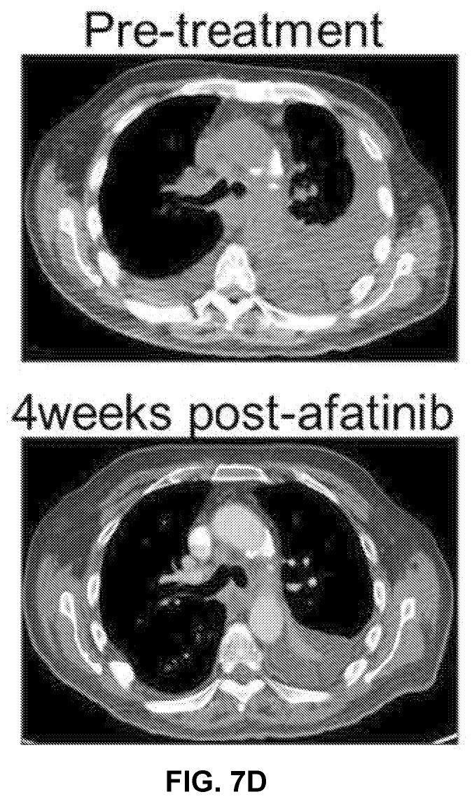

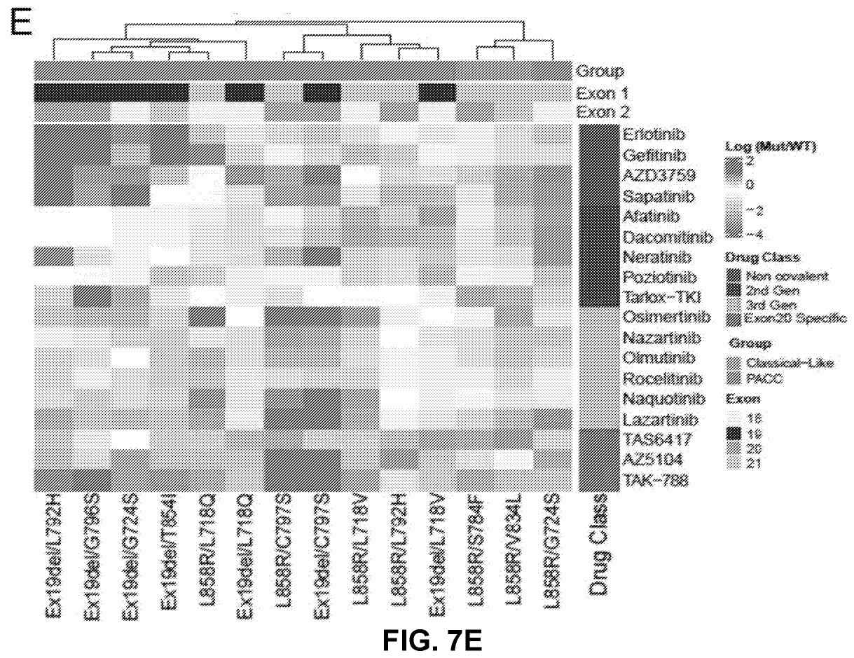

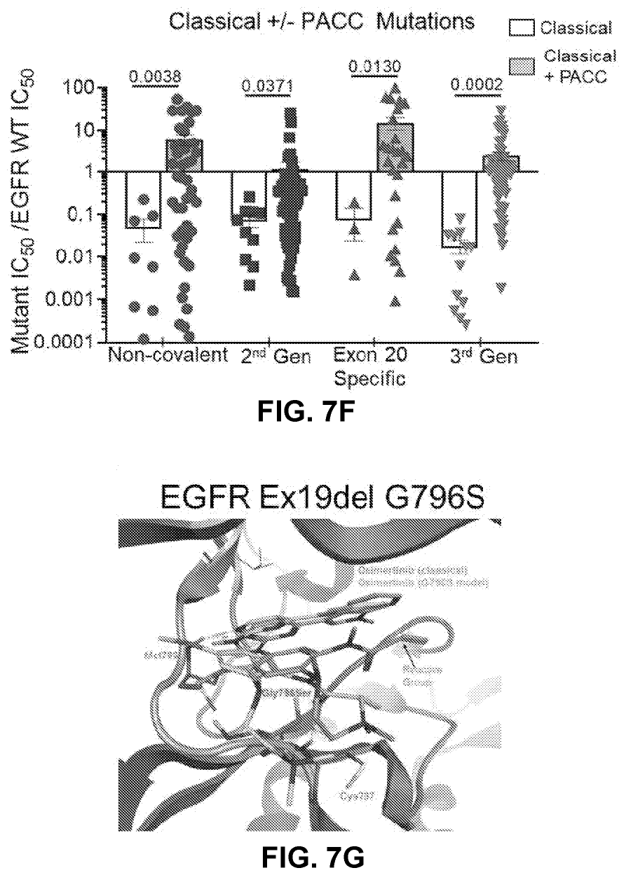

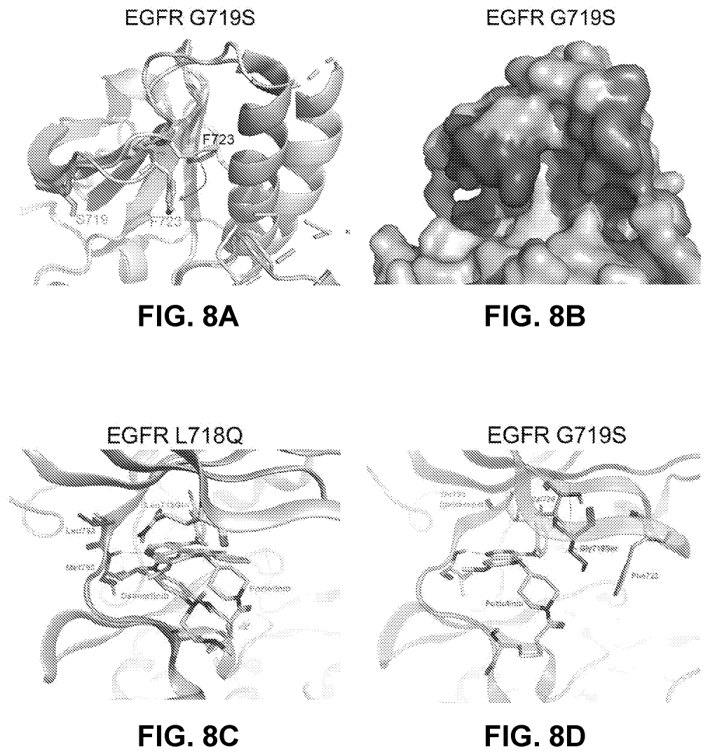

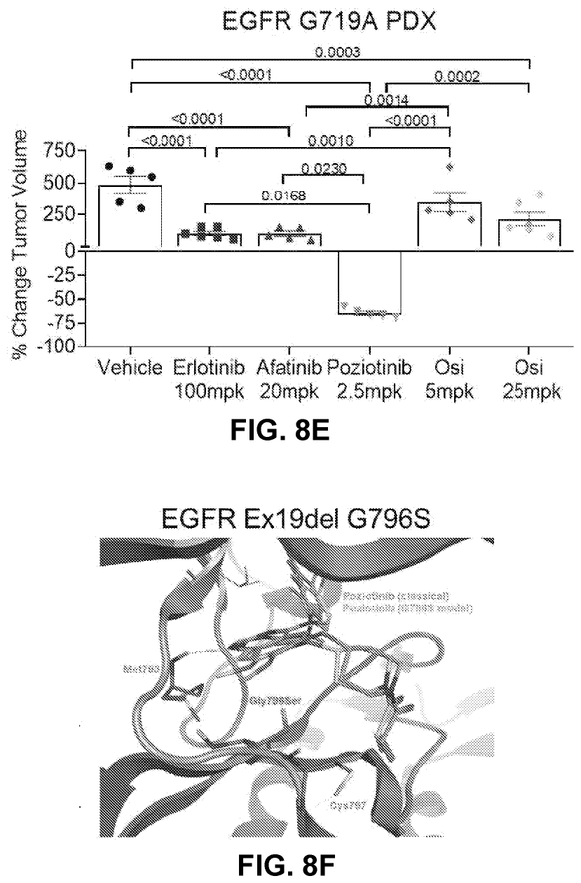

PACC mutations were comprised of mutations spanning exons 18-21 including mutations such as G719X, L747X, S768I, L792X, and T854I and others. PACC mutations were predicted to impact the overall volume of the ATP and drug binding pocket through alterations of the orientation of the P-loop or α-c-helix (FIG. 5A, FIG. 5B). In silico analysis of the interaction of osimertinib with PACC mutations G719S and L718Q predicted that changes in the orientation of the P-loop alter the position of TKI stabilization points such as V726 and F723 causing the indole ring of osimertinib to be tilted away from the P-loop compared to the reactive conformation of osimertinib, destabilizing drug binding (FIG. 7A, FIG. 5C). In contrast, second-generation EGFR TKIs do not interact with the P-loop of EGFR and maintain essential interaction points in the hydrophobic cleft in PACC mutants (FIG. 5C, FIG. 5D). When the selectivity of PACC mutations was compared to first, second, and third-generation, and ex20ins specific EGFR TKIs, it was found that second-generation EGFR TKIs were significantly more selective for PACC mutations than any other class of TKI (FIG. 7B). In vivo it was also observed that mice harboring PDXs with G719A mutations were resistant to the third-generation TKI, osimertinib but most sensitive to the second-generation EGFR TKI (FIG. 7C, FIG. 5E). Lastly, a patient with a complex PACC mutation, E709K G719S, saw clinical benefit and tumor shrinkage with afatinib treatment after progressing on osimertinib (FIG. 7D). Together these data demonstrated that PACC mutations are a distinct subgroup of EGFR mutations; are resistant to third-generation EGFR TKIs; and sensitive to second-generation EGFR TKIs.

Similarly, acquired PACC mutations co-occurring with primary classical EGFR mutations retained sensitivity to second-generation EGFR TKIs while acquiring resistance to third-generation EGFR TKIs (FIG. 7E, FIG. 7F). As previously described, allele specificity was observed in acquired drug resistance with acquired PACC mutations (FIG. 7E). In silico analysis of acquired mutations such as G796S co-occurring with Ex19del was predicted to confer resistance to third-generation EGFR TKIs such as osimertinib by shifting the hinge region of the receptor preventing stabilization of osimertinib at M793 and displacing the acrylamide group of osimertinib away from C797 thus preventing binding (FIG. 7G). However, second-generation inhibitors were less effected by shifts in the hinge region of the receptor and were predicted to maintain the orientation of the acrylamide group near C797 (FIG. 5F). Within the MD Anderson GEMINI database one patient was identified with lung adenocarcinomas harboring EGFR L858R mutations that received first-line osimertinib treatment and subsequently developed an EGFR-dependent mechanism of resistance. A PACC mutation was identified upon biopsy at progression (FIG. 6A, FIG. 6B). The patient acquired a L718V mutation, and was treated with a second-generation EGFR TKI and experienced clinical benefit of stable disease and tumor shrinkage (FIG. 6A, FIG. 6B). Taken together, these data demonstrate that both primary and acquired PACC mutations are sensitive to second-generation EGFR TKIs in preclinical models and in patients, and structure-function based groupings could identify a novel grouping of mutations for which an earlier generation of EGFR TKIs had the greatest selectivity.

### 4. Structure-Function Based Subgroups Predict Patient Outcomes to TKI Better than Exon-Based Subgroups

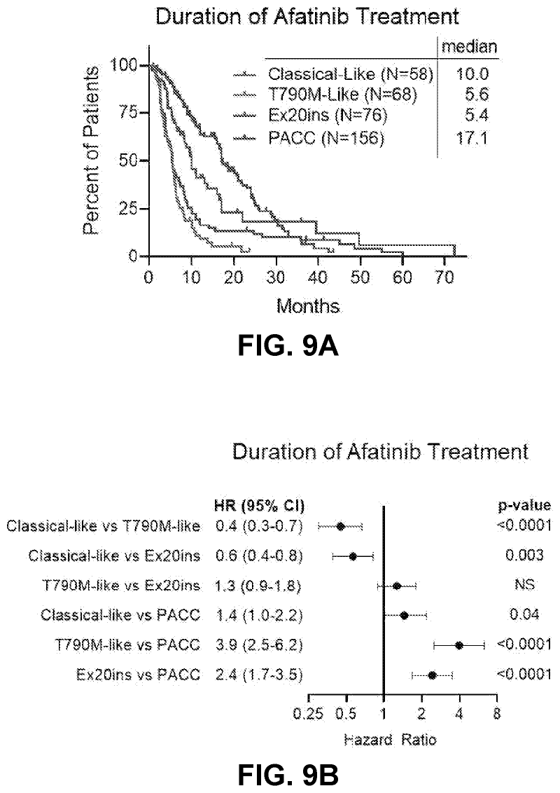

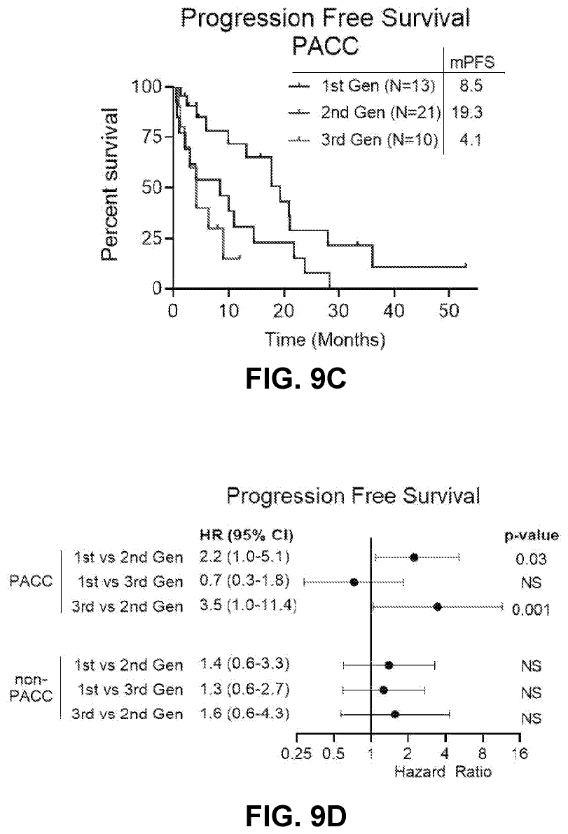

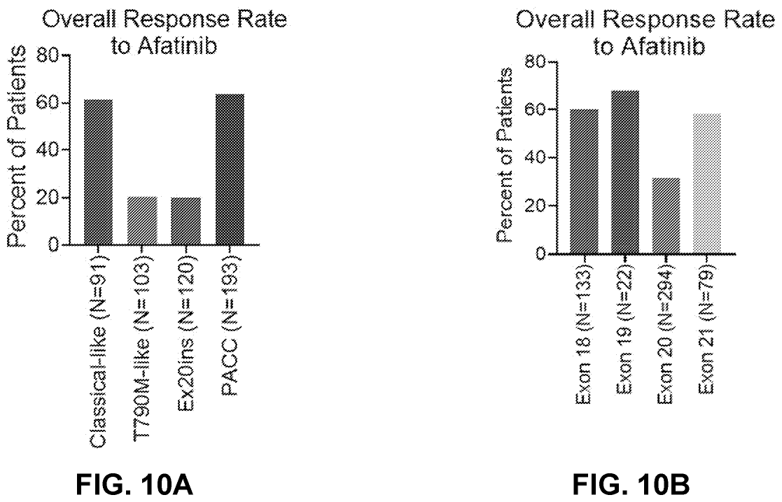

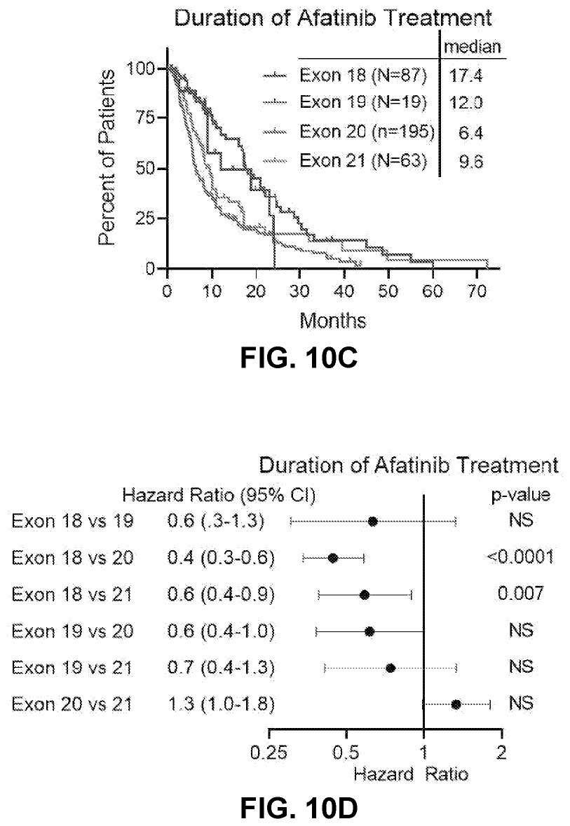

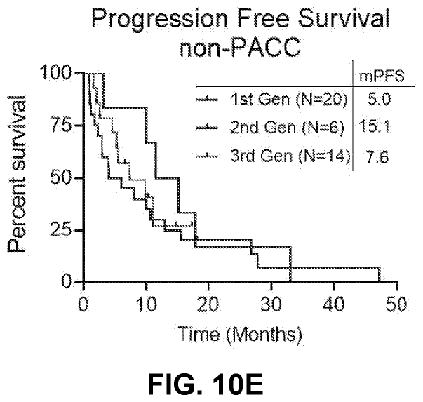

To determine if structure-function based groups could better identify patients most likely to benefit from a given drug compared to exon based groups, a publically available database of clinical outcomes from patients with NSCLC harboring atypical EGFR mutations treated with afatinib was used^{39,40}, and a retrospective analysis was performed of ORR and duration of treatment (DoT) of 847 patients. Structure-function based grouping showed clear differences between sensitive (classical-like and PACC) and resistant (T790M-like and Ex20ins) subgroups (ORR 63% vs 20%), whereas exon based groups had less variation between groups (FIG. 10A, FIG. 10B). Further structure-function based groups identified more subgroups of patients with a significantly longer DoT to afatinib treatment than exon based groups (FIG. 9A, FIG. 9B and FIG. 10C, FIG. 10D). Exon based groups identified that patients with exon 18 mutations had a longer DoT than patients with mutations in exons 20 or 21 (FIG. 10C, FIG. 10D). Whereas structure-function based groups identified that patients with PACC mutations experience a significantly longer DoT than any other subgroup and that patients with classical-like mutations had a significantly longer DoT than patients with exon 20 loop insertions or T790M-like mutations (FIG. 9A, FIG. 9B). These data demonstrate that structure based groupings better identify which groups of patients would receive the greatest benefit from a given drug than exon based groupings.

To determine if structure based groups could identify which class of inhibitors would provide the most benefit to patients with atypical EGFR mutations compared to traditional groupings, retrospective analyses were performed of mPFS of patients with atypical EGFR mutations treated with either first-, second-, or third-generation EGFR TKIs in the MD Anderson GEMINI database. To determine if structure-based groups could identify which class of inhibitors would provide the most benefit to patients with atypical EGFR mutations, retrospective analyses were performed of mPFS of patients with atypical EGFR mutations treated with either first-, second-, or third-generation EGFR TKIs in the MD Anderson GEMINI database. Patients with PACC mutations that were treated with second-generation EGFR TKIs had a significantly longer PFS than patients treated with either first- or third-generation EGFR TKIs (19.3 months vs. 8.5 and 4.1 months, respectively, FIG. 9C, FIG. 9D). By contrast, progression free survival was not significantly different between classes of EGFR TKIs in patients with atypical mutations that were non-PACC mutations (FIG. 10E), confirming that PACC mutations had a heightened sensitivity to second-generation EGFR TKIs as predicted by pre-clinical modeling. These data demonstrate that structure-based groupings could better identify which class of EGFR TKIs would provide the most benefit to patients with a particular group of mutations.

### 5. Conclusion

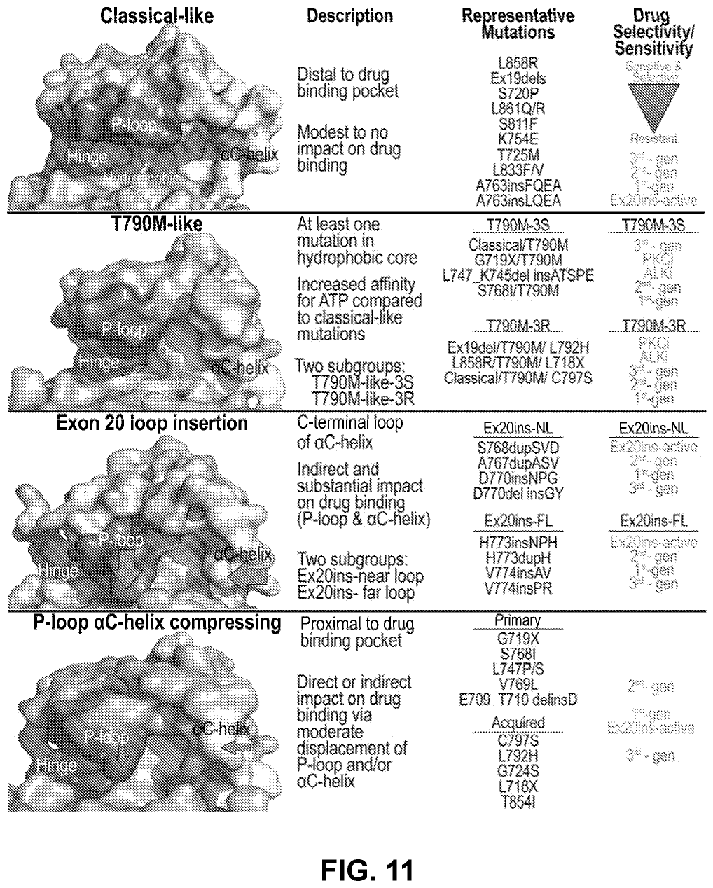

The diversity and higher than previously appreciated prevalence of atypical EGFR mutations highlights the necessity of comprehensive next generation sequencing (NGS) for patients with NSCLC. As described herein, EGFR mutations, including atypical mutations, can be divided into four distinct subgroups based on structure and function, and that structure/function-based groups can predict drug sensitivity and patient outcomes better than exon-based groups. These four subgroups are: “Classical-like,” “T790M-like,” “Exon 20 loop insertion,” and “P-loop αC-helix compressing,” (or “PACC”). The four subgroups, including description and example mutations, are provided in FIG. 11. “Exon 20 loop insertion” mutations are further separated into Exon 20 near-loop insertion (Es20ins-NL) and Exon 20 far-loop (Ex20ins-FL) mutations. “T790M-like” mutations are further separated into T790M-like-3S and T790M-like-3R mutations.

While previous studies have shown activity of second-generation EGFR TKIs in patients with select exon 18 mutations^{33,34}, structure/function-based grouping identified a larger subgroup of EGFR mutations, PACC mutants, for which second-generation EGFR TKIs were more selective than third-generation EGFR TKIs. Clinically, second-generation EGFR TKIs have been associated with WT EGFR inhibition and related adverse events^{15,35,36}; however, most second-generation EGFR TKIs are dosed at the maximum tolerated doses, resulting in plasma concentrations 10-100 fold greater than concentrations necessary for inhibiting PACC mutations. Unlike osimertinib, second-generation EGFR TKIs have limited CNS activity, demonstrating the need for novel EGFR TKIs with reduced WT EGFR inhibition and CNS activity that can inhibit PACC mutations.

These studies demonstrated that structure/function-based groups can identify classes of drugs that may be effective for whole groups of mutations, reflecting the observation that mutations in different regions of the gene may induce similar changes in protein structure. For example, L718Q, S768I, T854I are in exons 18, 20, and 21, respectively, but are all PACC mutations with similar structural impact on drug binding. Conversely, mutations within the same exon may induce quite disparate changes. L747_K754del-insATSPE, L747P, and E746-A750del mutations are in exon 19 but are T790M-like, PACC, and classical mutations, respectively, with distinct differences in drug sensitivity and structural impact. A clinical challenge for physicians treating patients with EGFR mutant cancers is to appropriately identify and match patient mutations with the best EGFR TKL. The classification presented here provides a framework through which clinicians, informed by internet-based tools or companies providing NGS reports, could more effectively personalize EGFR TKI therapy. Lastly, these findings support the notion that for cancers harboring oncogenes with diverse mutations, adopting a structure/function-based approach may improve clinical trial design and drug development.

### 6. Exemplary Methods

Ba/F3 cell generation, drug screening, and IC₅₀ approximations. Ba/F3 cells were obtained as a gift from Dr. Gordon Mills (MD Anderson Cancer Center), and maintained in RPMI (Sigma) containing 10% FBS, 1% penicillin/streptomycin, and 10 ng/ml recombinant mIL-3 (R&D Biosystems). To establish stable Ba/F3 cell lines, Ba/F3 cells were transduced with retroviruses containing mutant EGFR plasmids for 12-24 hours. Retroviruses were generated using Lipofectamine 2000 (Invitrogen) transfections of Phoenix 293T-ampho cells (Orbigen) with pBabe-Puro based vectors listed below in Table 6. Vectors were generated by GeneScript or Bioinnovatise using parental vectors from Addgene listed below in Table 6.

TABLE 6
EGFR Mutant Vectors Used to Generate Cell Lines

| | | | | Purchased/ |
|---|---|---|---|---|
| EGFR Mutation | Starting Vector | Genomic change | Manufacturer | Created |
| L858R L718Q | EGFR L858R | c.2153T > A | GeneScript | Created |
| L858R L718V | EGFR L858R | c.2152C > G | GeneScript | Created |
| L858R S784F | EGFR L858R | c.2351C > T | GeneScript | Created |
| L858R T790M L718Q | EGFR L858R T790M | c.2153T > A | GeneScript | Created |
| L858R T790M L718V | EGFR L858R T790M | c.2152C > G | GeneScript | Created |
| L858R T790M V843I | EGFR L858R T790M | c.2527G > A | GeneScript | Created |
| L861R | EGFR WT | c.2582T > G | GeneScript | Created |
| N771dupN G724S | EGFR G724S | c.2313_2314insAAC | GeneScript | Created |
| R776C | EGFR WT | c.2326C > T | GeneScript | Created |
| R776H | EGFR WT | c.2327G > A | GeneScript | Created |
| S720P | EGFR WT | c.2158T > C | GeneScript | Created |
| S768dupSVD V769M | EGFR S768dupSVD | c.2305G > A | GeneScript | Created |
| S768I V769L | EGFR S768I | c.2305G > T | GeneScript | Created |
| S768I V774M | EGFR S768I | c.2320G > A | GeneScript | Created |
| S784F | EGFR WT | c.2351C > T | GeneScript | Created |
| S811F | EGFR WT | c.2432C > T | GeneScript | Created |
| T725M | EGFR WT | c.2174C > T | GeneScript | Created |
| V774M | EGFR WT | c.2320G > A | GeneScript | Created |

After 48-72 hours of transduction, 2 μg/ml puromycin (Invitrogen) was added to Ba/F3 cell lines in complete RPMI. To select for EGFR positive cell lines, cells were stained with PE-EGFR (Biolegend) and sorted by FACS. After sorting, EGFR positive cells were maintained in RPMI containing 10% FBS, 1% penicillin/streptomycin, and 1 ng/ml EGF to support cell viability. Drug screening was performed as previously described^{41,42}. Shortly, cells were plated in 384-well plates (Greiner Bio-One) at 2000-3000 cells per well in technical triplicate. Seven different concentrations of TKIs or DMSO vehicle were added to reach a final volume of 40 μL per well. After 72 hours, 11 μL of Cell Titer Glo (Promega) was added to each well. Plates were incubated for a minimum of 10 minutes, and bioluminescence was determined using a FLUOstar OPTIMA plate reader (BMG LABTECH). Raw bioluminescence values were normalized to DMSO control treated cells, and values were plotted in GraphPad Prism. Non-linear regressions were used to fit the normalized data with a variable slope, and IC₅₀ values were determined by GraphPad prism by interpolation of concentrations at 50% inhibition. Drug screens were performed in technical triplicate on each plate and either duplicate or triplicate biological replicates. Mutant to WT ratios (Mut/WT) for each drug were calculated by dividing the IC₅₀ values of mutant cell lines by the average IC₅₀ value of Ba/F3 cells expressing WT EGFR supplemented with 10 ng/ml EGF for each drug. Statistical differences between groups were determined by ANOVA as described in the figure legends. Table 7 shows a summary of all of the drugs tested.

TABLE 7
Summary of Compounds Tested

| | | Primary | | |
|---|---|---|---|---|
| EGFR TKI | Class | Target | Binding | Structure |
| Erlotinib | first- generation | EGFR | non- covalent | 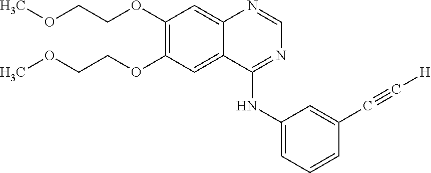 [SMILES: C#Cc1cccc(Nc2ncnc3cc(OCCOC)c(OCCOC)cc23)c1] |
| Gefitinib | first- generation | EGFR | non- covalent | 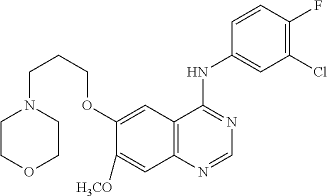 [SMILES: COc1cc2ncnc(Nc3ccc(F)c(Cl)c3)c2cc1OCCCN1CCOCC1] |
| AZD3759 | first- generation | EGFR | non- covalent | 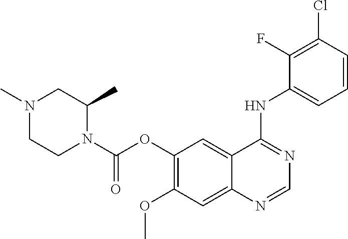 [SMILES: COc1cc2ncnc(Nc3cccc(Cl)c3F)c2cc1OC(=O)N1CCN(C)C[C@H]1C] |
| Sapatinib | first- generation | EGFR | non- covalent | 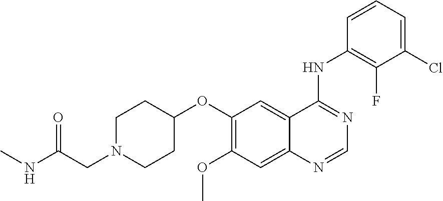 [SMILES: CNC(=O)CN1CCC(Oc2cc3c(Nc4cccc(Cl)c4F)ncnc3cc2OC)CC1] |
| Afatinib | second- generation | EGFR | covalent | 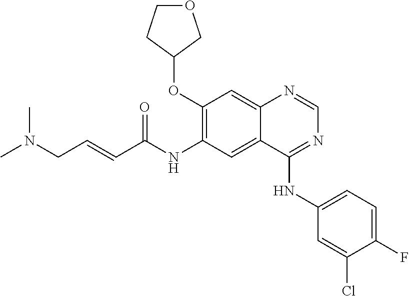 [SMILES: CN(C)C/C=C/C(=O)Nc1cc2c(Nc3ccc(F)c(Cl)c3)ncnc2cc1OC1CCOC1] |
| Dacomitinib | second- generation | EGFR | covalent | 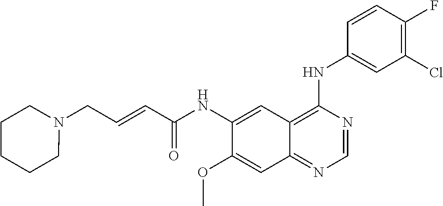 [SMILES: COc1cc2ncnc(Nc3ccc(F)c(Cl)c3)c2cc1NC(=O)/C=C/CN1CCCCC1] |
| Neratinib | second- generation | EGFR | covalent | 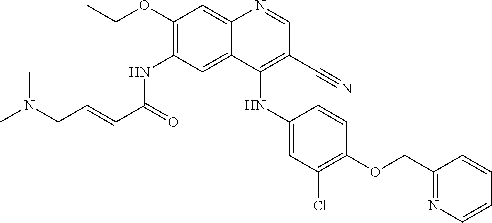 [SMILES: CCOc1cc2ncc(C#N)c(Nc3ccc(OCc4ccccn4)c(Cl)c3)c2cc1NC(=O)/C=C/CN(C)C] |
| Tarlox-TKI | second- generation | EGFR | covalent | 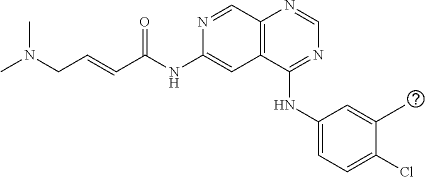 [SMILES: Cc1cc(Nc2ncnc3cnc(NC(=O)/C=C/CN(C)C)cc23)ccc1Cl] |
| CLN-081 | Ex20ins Specific | EGFR | covalent | 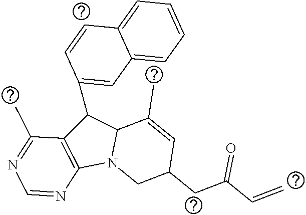 [SMILES: C=CC(=O)CC1C=C(C)C2C(c3ccc4ccccc4c3)c3c(C)ncnc3N2C1] |
| AZ5104 | Ex20ins Specific | EGFR | covalent | 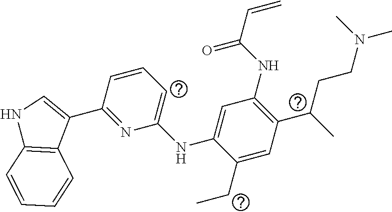 [SMILES: C=CC(=O)Nc1cc(Nc2cccc(-c3c[nH]c4ccccc34)n2)c(CC)cc1C(C)CCN(C)C] |
| Mobocertinib | Ex20ins Specific | EGFR | covalent | 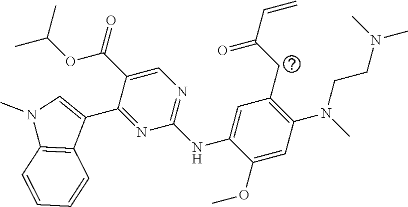 |
| Osimertinib | third- generation | EGFR | covalent | 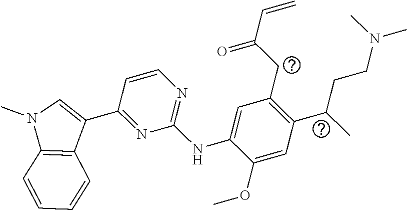 |
| Nazartinib | third- generation | EGFR | covalent | 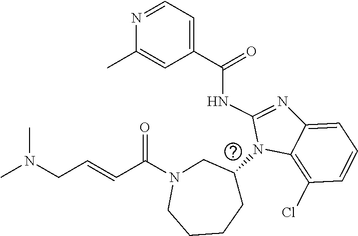 [SMILES: Cc1cc(C(=O)Nc2nc3cccc(Cl)c3n2[C@@H]2CCCCN(C(=O)/C=C/CN(C)C)C2)ccn1] |
| Olmutinib | third- generation | EGFR | covalent | 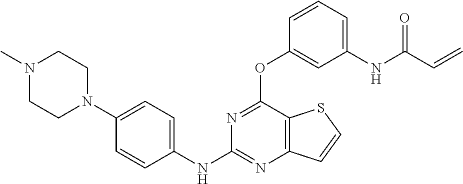 [SMILES: C=CC(=O)Nc1cccc(Oc2nc(Nc3ccc(N4CCN(C)CC4)cc3)nc3ccsc23)c1] |
| Rociletinib | third- generation | EGFR | covalent | 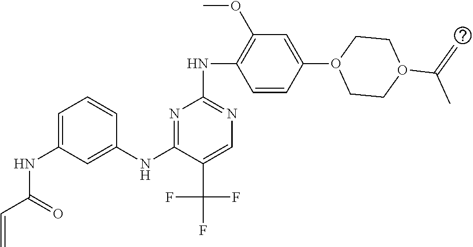 |
| Naquotinib | third- generation | EGFR | covalent | 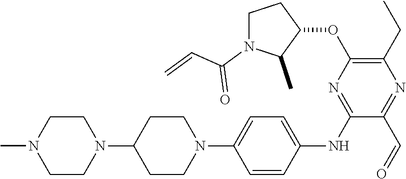 |
| Lazertinib | third- generation | EGFR | covalent | 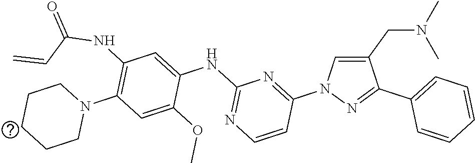 [SMILES: C=CC(=O)Nc1cc(Nc2nccc(-n3cc(CN(C)C)c(-c4ccccc4)n3)n2)c(OC)cc1N1CCCCC1] |
| Ruboxistaurin | PKC | PKC | non- covalent | 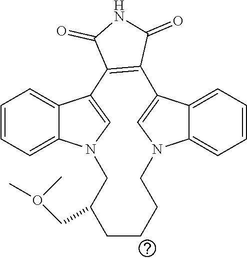 |
| Sotrastaurin | PKC | PKC | non- covalent | 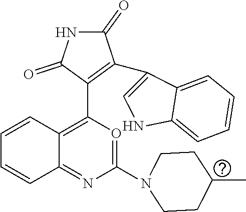 |
| Midostaurin | PKC | PKC | non- covalent | 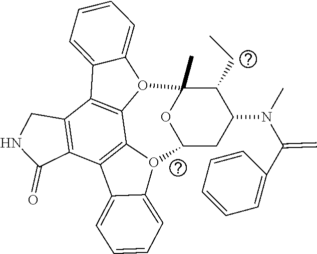 |
| CUDC-101 | EGFR/HDAC | EGFR/ HDAC | non- covalent | 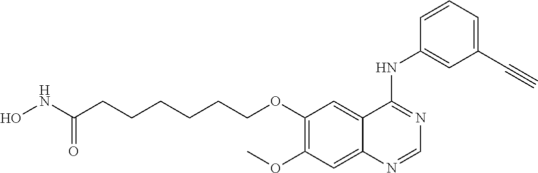 [SMILES: C#Cc1cccc(Nc2ncnc3cc(OC)c(OCCCCCCC(=O)NO)cc23)c1] |
| Brigatinib | ALK | ALK | non- covalent | 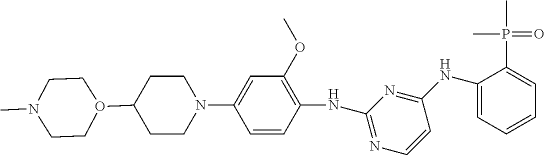 |
| AZD3463 | ALK | ALK | non- covalent | 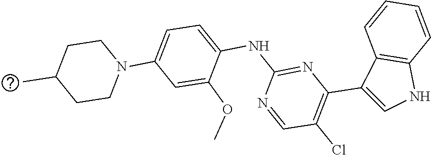 [SMILES: COc1cc(N2CCC(C)CC2)ccc1Nc1ncc(Cl)c(-c2c[nH]c3ccccc23)n1] |

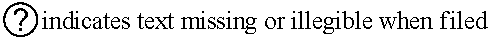

In silico mutational mapping and docking experiments. X-ray structures of wild type EGFR in complex with AMP-PNP (2ITX) and EGFR^{L858R} mutant in complex with AMP-PNP (2ITV) retrieved from PDB was used for MD simulation. All crystallographic ligands, ions, and water molecules were removed from the X-ray structures. Missing side-chain atoms and loops in these structures were built using the Prime homology module⁴³ in Schrodinger. The missing activation loop region (862-876) in the EGFR^{L858R} mutant structure was built using the activation loop from another EGFR structure (5XGN). Exon 19 deletion mutant (ΔELREA) was modeled on the wild type EGFR, using the Prime program, followed by MM/GBSA based loop refinement for the β3-αC loop region. Sidechain prediction for all the double mutants (EGFR^{L858R/L718Q}, EGFR^{Ex19del/L718Q}, EGFR^{L858R/L792H} EGFR^{Ex19del/L792H}) was carried out using the Prime side-chain prediction in Schrodinger, employing backbone sampling, followed by minimization of the mutated residue. The structures were finally prepared using the “QuickPrep” module in MOE⁴⁴. Pymol software was used for visualization of mutation location on WT (2ITX) EGFR, and alignment with EGFR D770insNPG (4LRM) or EGFR G719S (2ITN).

Heatmap generation and spearman correlations of groups. Heat maps and hierarchical clustering were generated by plotting the median log (Mut/WT) value for each cell line and each drug using R and the ComplexHeatmap package (R Foundation for Statistical Computing, Vienna, Austria. Complex Heatmap)⁴⁵. Hierarchical clustering was determined by Euclidean distance between Mut/WT ratios. For co-occurring mutations, exon order was assigned arbitrarily, and for acquired mutations, exons were assigned in the order mutations are observed clinically. Structure-function groups were assigned based on predicted impact of mutation on receptor conformation. Correlations for mutations were determined using Spearman's rho by correlating the median log (Mut/WT) value for each mutation and drug versus the average of the median log (Mut/WT) value for the structure-function based group or exon based group for which the mutation belongs. For each correlation, the mutation tested was removed from the average structure function and exon based groups. Average rho values were compared by two-sided students' t-test.

PDX generation and in vivo experiments. As part of the MD Anderson Cancer Center Lung Cancer Moon Shots program, patient derived xenografts were generated and maintained in accordance with Good Animal Practices and with approval from MD Anderson Cancer Center Institutional Animal Care and Use Committee (Houston, TX) on protocol number PA140276 as previously described⁴⁶. Surgical samples were rinsed with serum-free RPMI supplemented with 1% penicillin-streptomycin then implanted into the right flank of 5- to 5-week old NSG mice within two hours of resection. Tumors were validated for EGFR mutations by DNA fingerprinting and qPCR as described⁴⁶. To propagate tumors, 5- to 6-week old female NSG mice (NOD.Cg-Prkdcscid IL2rgtmWjl/Szj) were purchased from Jax Labs (#005557). Fragments of NSCLC tumors expressing EGFR G719S or L858R/E709K were implanted into 6-8 week old female NSG mice. Once tumors reached 2000 mm³, tumors were harvest and re-implanted into the right flank of 6-8 week old female NSG mice. Tumors were measured three times per week, and were randomized into treatment groups when tumors reached a volume of 275-325 mm³ for the EGFR G719S model, and 150-175 mm³ for the L858R/E709K model. Treatment groups included vehicle control (0.5% Methylcellulose, 0.05% Tween-80 in dH₂O), 100 mg/kg erlotinib, 20 mg/kg afatinib, 5 mg/kg osimertinib, and 20 mg/kg osimertinib. During treatment, body eight and tumor volumes were measured three times per week, and mice received treatment five days per week (Monday-Friday). Dosing holidays were given if mouse body weight decreased by more than 10% or overall body weight dropped below 20 grams.

Case studies of patients treated with second-generation TKIs. Patients were consented under the GEMINI protocol (PA13-0589) which was approved in accordance with the MD Anderson Institutional Review Board.

Retrospective analysis of ORR and duration of treatment with afatinib. Response to afatinib and duration of afatinib treatment was tabulated from 803 patients in the Uncommon EGFR Database³⁹. Objective response rate was reported in 529 patients. Patients were stratified by either structure-function based groups or exon based groups and ORR was determined by counting the number of patients reported to have complete response or partial response. Fisher's exact test was used to determined statistical differences between subgroups (structure based or exon based). Duration of treatment was provided in the Uncommon EGFR database for 746 patients. Patients were stratified by structure-function based groups and exon based groups and median DoT was calculated using the Kaplan-Meier method. Statistical differences in Kaplan-Meier plots, hazard ratios, and p-values were generated using GraphPad prism software and the Mantel-Cox Log-Rank method. When mutations were not explicitly stated (i.e. exon 19 mutation) those patients were excluded from the structure-function based analysis but included in the exon based analysis.

Retrospective analysis of PFS of patients with atypical mutations. There were 333 patients with NSCLC identified in the MD Anderson GEMINI database that had tumors expressing atypical mutations. Of these patients, 81 patients received at least one line of EGFR tyrosine kinase inhibitor treatment and did not harbor an exon 20 loop insertion mutation. Clinical parameters were extracted from the respective databases. Patients previously receiving chemotherapy were included, and PFS was calculated for the first EGFR TKI received. PFS was defined as time from commencement of first EGFR TKI to radiologic progression or death. Median PFS was calculated using the Kaplan-Meier method and hazard ratios and p-values were calculated using Mantel-Cox Log-Rank method.

Tables 8.1-8.4 list EGFR mutations analyzed in the described studies, as well as assigned subgroups. An EGFR mutation of the present disclosure may be, without limitation, a mutation listed in Table 8.1, Table 8.2, Table 8.3, or Table 8.4.

TABLE 8.1
List of Classical-like EGFR mutations
Classical-Like Mutations

A702T
A763insFQEA
A763insLQEA
D761N
E709A L858R
E709K L858R
E746_A750del A647T
E746_A750del L41W
E746_A750del R451H
Ex19del E746_A750del
K754E
L747_E749del A750P
L747_T751del L861Q
L833F
L833V
L858R
L858R A289V
L858R E709V
L858R L833F
L858R P100T
L858R P848L
L858R R108K
L858R R324H
L858R R324L
L858R S784F
L858R S784Y
L858R T725M
L858R V834L
L861Q
L861R
S720P
S784F
S811F
T725M

TABLE 8.2
List of PACC EGFR mutations
PACC Mutations

A750_I759del insPN
E709_T710del insD
E709A
E709A G719A
E709A G719S
E709K
E709K G719S
E736K
E746_A750del A647T
E746_A750del R675W
E746_T751del insV S768C
Ex19del C797S
Ex19del G796S
Ex19del L792H
Ex19del T8541
G719A
G719A D761Y
G719A L861Q
G719A R776C
G719A S768I
G719A S768I
G719C S768I
G719S
G719S L861Q
G719S S7681
G724S
G724S Ex19del
G724S L858R
G779F
I740dupIPVAK
K757M L858R
K757R
L718Q
L718Q Ex19del
L718Q L858R
L718V
L718V Ex19del
L718V L858R
L747_S752del A755D
L747P
L747S
L747S L858R
L747S V774M
L858R C797S
L858R L792H
L858R T854S
N771G
R776C
R776H
E709_T710del insD S22R
S752_I759del V769M
S768I
S768I L858R
S768I L861Q
S768I V769L
S768I V774M
T751_I759 delinsN
V769L
V769M
V774M

TABLE 8.3
List of Exon20 Loop Insertion EGFR mutations
Ex20 Loop Insertions

| Near-loop | Far-loop |
|---|---|
| A767_V769dupASV | H773_V774 insNPH |
| A767_S768insTLA | H773_V774 insAH |
| S768_D770dupSVD | H773dupH |
| S768_D770dupSVD L858Q | V774_C775 insHV |
| S768_D770dupSVD R958H | V774_C775 insPR |
| S768_D770dupSVD V769M | |
| V769_D770insASV | |
| V769_D770insGSV | |
| V769_D770insGVV | |
| V769_D770insMASVD | |
| D770_N771insNPG | |
| D770_N771insSVD | |
| D770del insGY | |
| D770_N771 insG | |
| D770 N771 insY H773Y | |
| N771dupN | |
| N771dupN G724S | |
| N771_P772insHH | |
| N771_P772insSVDNR | |
| P772_H773insDNP | |

TABLE 8.4
List of T790M-like EGFR mutations
T790M-like Mutations

| T790M-like-3S | T790M-like-3R |
|---|---|
| Ex19del T790M | Ex19del T790M C797S |
| Ex19del T790M L718V | Ex19del T790M L792H |
| Ex19del T790M G724S | G724S T790M |
| G719A T790M | L718Q T790M |
| G719S T790M | L858R T790M C797S |
| H773R T790M | L858R T790M L718Q |
| I744_E749del insMKK | L858R T790M L718V |
| L747_K754 delinsATSPE | |
| L858R T790M L792H | |
| L858R T790M V843I | |
| L858R T790M | |
| S768I T790M | |
| T790M | |

TABLE 9
Patient Characteristics

| | | | | | Tobacco | | | Previous | | Stage |
|---|---|---|---|---|---|---|---|---|---|---|
| Mutation | | EGFR | Age | | Use | Pack | Histo | Chemo | | at |
| Type | Exon | Mutation | (years) | Sex | (Y/N) | Years | Pathology | (Y/N) | TKI | diagnosis |
| Classical | 19 | E746_A750 | 73 | M | Y | Unknown | Adenocarcinoma | N | Erlotinib | IV |
| Like | | A750E | | | | | | | | |
| Classical | 21 | L747_T751del | 59 | F | Y | 5 | Adenocarcinoma | N | Afatinib | IV |
| Like | | L861Q | | | | | | | | |
| Classical | 21 | L861Q | 70 | F | N | N/A | Adenocarcinoma | N | Afatinib | II |
| Like | | | | | | | | | | |
| Classical | 21 | L858R E709K | 59 | F | Y | 10 | Adenocarcinoma | N | Afatinib | IV |
| Like | | | | | | | | | | |
| Classical | 21 | L858R P848L | 74 | M | N | N/A | Adenocarcinoma | N | Osimertinib | IV |
| Like | | | | | | | | | | |
| Classical | 18 | L858R T725M | 52 | F | N | N/A | Adenocarcinoma | N | Osimertinib | IV |
| Like | | | | | | | | | | |
| Classical | 21 | L861Q | 77 | M | N | N/A | Adenocarcinoma | N | Osimertinib | IV |
| Like | | | | | | | | | | |
| Classical | 21 | L861Q | 78 | M | N | N/A | Adenocarcinoma | Y | Erlotinib | IV |
| Like | | | | | | | | | | |
| Classical | 18 | T725M | 75 | F | N | N/a | Adenocarcinoma | Y | Gefitinib | IV |
| Like | | | | | | | | | | |
| Classical | 18 | A702T | 62 | M | Y | 47 | Adenocarcinoma | Y | Afatinib | IV |
| Like | | | | | | | | | | |
| Classical | 21 | S811F | 84 | F | Y | 37 | Adenocarcinoma | Y | erlotinib | IV |
| Like | | | | | | | | | | |
| Classical | 19 | L747_E749del | 81 | F | Y | 8 | Adenocarcinoma | N | Erlotinib | I |
| Like | | A750P | | | | | | | | |
| Classical | 21 | L861Q | 70 | M | Y | 24 | Adenocarcinoma | N | Erlotinib | IV |
| Like | | | | | | | | | | |
| Classical | 21 | L861Q | 73 | F | N | N/A | Adenocarcinoma | N | Erlotinib | I |
| Like | | | | | | | | | | |
| Classical | 21 | L858R L833F | 82 | M | Y | 51 | Adenocarcinoma | Y | Erlotinib | IV |
| Like | | | | | | | | | | |
| Classical | 18 | L858R E709V | 60 | M | N | N/A | Adenocarcinoma | Y | Rociletinib | IV |
| Like | | | | | | | | | | |
| Classical | 19 | E746_A750del | 56 | M | N | N/A | Adenocarcinoma | Y | Erlotinib | IV |
| Like | | A647T | | | | | | | | |
| Classical | 21 | E709K L858R | 70 | M | Y | 5 | Adenocarcinoma | Y | Erlotinib | IV |
| Like | | | | | | | | | | |
| Classical | 21 | L861Q | 82 | F | Y | Unknown | Adenocarcinoma | N | Erlotinib | IV |
| Like | | | | | | | | | | |
| Classical | 18 | E709K L858R | 45 | M | N | N/A | Adenocarcinoma | N | Rociletinib | IV |
| Like | | | | | | | | | | |
| Classical | 21 | L861Q | 60 | M | Y | 90 | Adenocarcinoma | N | Gefitinib | IV |
| Like | | | | | | | | | | |
| Classical | 21 | E709K/L858R | 83 | F | N | N/A | Adenocarcinoma | N | Afatinib | IV |
| Like | | | | | | | | | | |
| Classical | 19 | L747_E749del | 45 | M | Y | 5 | Adenocarcinoma | Y | Osimertinib | III |
| Like | | A750P | | | | | | | | |
| Classical | 21 | L861Q | 77 | F | Y | 10 | Adenocarcinoma | N | Erlotinib | IV |
| Like | | | | | | | | | | |
| Classical | 19 | D761Y | 74 | M | Y | 35 | Squamous cell | Y | Afatinib | IV |
| Like | | | | | | | carcinoma | | | |
| Classical | 21 | L858R S784F | 71 | F | N | N/A | Adenocarcinoma | N | Osimertinib | IV |
| Like | | | | | | | | | | |
| Classical | 21 | L858R V834L | 75 | F | Y | 20 | Adenocarcinoma | Y | Osimertinib | III |
| Like | | | | | | | | | | |
| Classical | 21 | L858R L833V | 60 | F | N | N/A | Adenocarcinoma | Y | Osimertinib | IV |
| Like | | | | | | | | | | |
| Classical | 21 | L861Q | 71 | F | N | N/A | Adenocarcinoma | N | Osimertinib | IV |
| Like | | | | | | | | | | |
| Classical | 18 | E709A L858R | 69 | M | N | N/A | Adenocarcinoma | Y | Osimertinib | IV |
| Like | | | | | | | | | | |
| Classical | 21 | L861Q | 79 | M | N | N/A | Adenocarcinoma | N | Osimertinib | IV |
| Like | | | | | | | | | | |
| Classical | 21 | L858R S784Y | 53 | M | Y | 15 | Adenocarcinoma | N | Osimertinib | IV |
| Like | | | | | | | | | | |
| Classical | 6 | L858R R324L | 71 | F | Y | 32 | Adenocarcinoma | Y | Erlotinib | IV |
| Like | | | | | | | | | | |
| Classical | 5 | L858R A289V | 57 | M | N | N/A | Adenocarcinoma | N | Erlotinib | IV |
| Like | | | | | | | | | | |
| Classical | 6 | L858R R324H | 70 | F | N | N/A | Adenocarcinoma | Y | Erlotinib | IV |
| Like | | | | | | | | | | |
| Classical | 1 | L858R R108K | 73 | F | N | N/A | Adenocarcinoma | N | Erlotinib | IV |
| Like | | | | | | | | | | |
| Classical | 1 | E746_A750del | 59 | F | N | N/A | Adenocarcinoma | N | Gefitinib | IV |
| Like | | L41W | | | | | | | | |
| Classical | 1 | L858R P100T | 65 | M | Y | 43 | Adenocarcinoma | N | Gefitinib | IV |
| Like | | | | | | | | | | |
| Classical | 9 | E746_A750del | 57 | F | N | N/A | Adenocarcinoma | N | Erlotinib | IV |
| Like | | R451H | | | | | | | | |
| Ex20 | 20 | Exon 20 | 63 | F | N | N/A | Adenocarcinoma | N | Osimertinib | IV |
| LoopIns | | insertion | | | | | | | | |
| Ex20 | 20 | H773_V774ins | 60 | M | N | NA | Adenocarcinoma | Y | Erlotinib | IV |
| LoopIns | | H | | | | | | | | |
| Ex20 | 20 | A767_v769dup | 61 | F | N | NA | Adenocarcinoma | Y | Erlotinib | IV |
| LoopIns | | ASV | | | | | | | | |
| Ex20 | 20 | D770_N771ins | 49 | F | N | NA | Adenocarcinoma | Y | Afatinib | IV |
| LoopIns | | G | | | | | | | | |
| Ex20 | 20 | N771delinsGY | 79 | M | N | NA | Adenocarcinoma | Y | Erlotinib | IV |
| LoopIns | | | | | | | | | | |
| Ex20 | 20 | S768_D770dup | 47 | F | N | NA | Adenocarcinoma | N | Erlotinib | IV |
| LoopIns | | SVD | | | | | | | | |
| Ex20 | 20 | A767_V769dup | 69 | M | Y | 35 | Adenocarcinoma | Y | Afatinib | IV |
| LoopIns | | ASV | | | | | | | | |
| Ex20 | 20 | S768_D770dup | 65 | M | Y | 0.75 | Adenocarcinoma | N | Erlotinib | IV |
| LoopIns | | SVD | | | | | | | | |
| Ex20 | 20 | S768_V769deli | 57 | F | Y | <5 | Adenocarcinoma | Y | Afatinib | IV |
| LoopIns | | nsIL | | | | | | | | |
| Ex20 | 20 | S768_D770dup | 56 | F | N | NA | Adenocarcinoma | N | Erlotinib | IV |
| LoopIns | | SVD | | | | | | | | |
| PACC | 18 | G719S | 65 | F | Y | 60 | Adenocarcinoma | N | Osimertinib | III |
| PACC | 18 | G719A S768I | 47 | M | N | N/A | Adenocarcinoma | N | Erlotinib | IV |
| PACC | 18 | G719S S768I | 43 | F | Y | 2 | Adenocarcinoma | N | Afatinib | I |
| PACC | 19 | L858R K757M | 60 | F | Y | 5 | Adenocarcinoma | N | Erlotinib | I |
| PACC | 18 | G719S L861Q | 55 | F | Y | 10 | Adenocarcinoma | Y | Gefitinib | III |
| PACC | 18 | G719A | 85 | M | Y | 50 | Adenocarcinoma | N | Afatinib | IV |
| PACC | 18 | G719A | 66 | F | Y | 12 | Adenocarcinoma | N | Osimertinib | IV |
| PACC | 19 | L747_S752del | 62 | F | Y | 25 | Adenocarcinoma | N | Osimertinib | IV |
| | | A755D | | | | | | | | |
| PACC | 20 | V769M | 89 | M | Y | 45 | Adenosquamous | N | Osimertinib | IV |
| PACC | 18 | E709_T719del | 78 | F | N | N/A | Adenocarcinoma | N | Afatinib | IV |
| | | insD | | | | | | | | |
| PACC | 18 | G719A | 62 | M | Y | 10 | Adenocarcinoma | Y | Erlotinib | IV |
| PACC | 21 | L858R T854S | 71 | F | Y | Unknown | Adenosquamous | N | Afatinib | IV |
| PACC | 18 | G719C S768I | 70 | F | Y | 15 | Adenocarcinoma | N | Erlotinib | IV |
| PACC | 20 | L858R S768I | 82 | M | Y | 45 | Adenocarcinoma | Y | Afatinib | III |
| PACC | 20 | L858R S768I | 85 | F | N | N/A | Adenocarcinoma | N | Osimertinib | IV |
| PACC | 18 | G719A | 78 | M | Y | 24 | Adenocarcinoma | N | Afatinib | IV |
| PACC | 18 | G719S S768I | 74 | M | N | N/A | Adenocarcinoma | N | Erlotinib | IV |
| PACC | 19 | L858R L747S | 69 | M | N | N/A | Adenocarcinoma | N | Erlotinib | IV |
| PACC | 20 | E746_T751deli | 74 | F | N | N/A | Adenocarcinoma | N | Erlotinib | IV |
| | | nsV S768C | | | | | | | | |
| PACC | 18 | E709A G719A | 74 | M | Y | chewing | Adenocarcinoma | Y | Afatinib | IV |
| | | | | | | tobacco | | | | |
| PACC | 20 | S752_I759del | 64 | M | N | N/A | Adenocarcinoma | Y | Erlotinib | IV |
| | | V769M | | | | | | | | |
| PACC | 18 | G719S S768I | 75 | F | N | N/A | Adenocarcinoma | N | Erlotinib | IV |
| PACC | 18 | G719A S768I | 60 | M | Y | 90 | Adenocarcinoma | Y | Erlotinib | IV |
| PACC | 18 | G719A S768I | 66 | M | N | N/A | Adenocarcinoma | Y | Avitinib | IV |
| PACC | 18 | E709_T710deli | 80 | M | Y | 22 | Adenocarcinoma | Y | Afatinib | I |
| | | nsD S22R | | | | | | | | |
| PACC | 19 | E746_A750del | 66 | M | N | N/A | Adenocarcinoma | Y | Erlotinib | IV |
| | | R675W | | | | | | | | |
| PACC | 18 | G719A | 69 | F | N | N/A | Adenocarcinoma | N | Erlotinib | IV |
| PACC | 19 | E736K | 59 | F | Y | 14.5 | Adenocarcinoma | Y | Gefitinib | III |
| PACC | 18 | G719A S768I | 75 | F | Y | 17 | Adenocarcinoma | Y | Afatinib | III |
| PACC | 18 | E709_T710deli | 66 | M | N | N/A | Adenocarcinoma | Y | Afatinib | III |
| | | nsD | | | | | | | | |
| PACC | 18 | G719A | 66 | M | Y | 25 | Adenocarcinoma | Y | Afatinib | III |
| PACC | 18 | G719A | 68 | F | Y | 25 | Adenocarcinoma | N | Afatinib | III |
| PACC | 19 | T751_I759deli | 59 | F | N | N/A | Adenocarcinoma | Y | Gefitinib | IV |
| | | nsN | | | | | | | | |
| PACC | 20 | L861Q S768I | 56 | F | Y | 38 | Adenocarcinoma | Y | Afatinib | IV |
| PACC | 19 | L747S V774M | 49 | F | Y | 15 | Adenocarcinoma | N | Afatinib | IV |
| PACC | 20 | G719A R776C | 52 | F | N | N/A | Adenocarcinoma | N | Erlotinib | IV |
| PACC | 18 | E709K G719S | 62 | F | N | N/A | Adenocarcinoma | N | Afatinib | IV |
| PACC | 18 | G719A | 87 | F | Y | 17.5 | Adenocarcinoma | Y | Osimertinib | IV |
| PACC | 18 | E709K G719S | 84 | F | N | N/A | Adenocarcinoma | Y | Afatinib | IV |
| PACC | 18 | G719A | 67 | M | N | N/A | Adenosquamous | N | Afatinib | III |
| PACC | 20 | G779F | 71 | F | Y | 24 | Adenocarcinoma | Y | Afatinib | IV |
| PACC | 19 | T751_I759deli | 74 | F | Y | 1 | Adenocarcinoma | Y | Osimertinib | IV |
| | | nsN | | | | | | | | |
| PACC | 19 | L747P | 63 | F | N | N/A | Adenocarcinoma | N | Osimertinib | I |
| PACC | 18 | G719S S768I | 62 | M | N | N/A | Adenocarcinoma | Y | Afatinib | IV |
| PACC | 19 | A750_I759deli | 51 | F | Y | 13 | Squamous cell | Y | Osimertinib | IV |
| | | nsPN | | | | | carcinoma | | | |
| PACC | 18 | G719A D761Y | 67 | F | Y | 32 | Adenocarcinoma | Y | Osimertinib | II |
| PACC | 20 | S768I | 64 | F | Y | 15 | Adenocarcinoma | N | Afatinib | IV |
| PACC | 18 | E709A G719S | 45 | F | N | N/A | Adenocarcinoma | N | Afatinib | IV |
| PACC | 18 | G719S L861Q | 66 | M | N | N/A | Adenocarcinoma | N | Afatinib | IV |
| PACC | 18 | G719A | 68 | F | N | N/A | Adenocarcinoma | Y | erlotinib | IV |
| PACC | 18 | G719A | 71 | F | N | N/A | Large | N | Afatinib | IV |
| | | | | | | | Neuroendocrine | | | |
| T790M- | 18 | G719S T790M | 62 | F | N | N/A | Adenocarcinoma | N | Osimertinib | IV |
| like-3S | | | | | | | | | | |
| T790M- | 20 | H773R T790M | 50 | F | N | N/A | Adenocarcinoma | Y | Osimertinib | IV |
| like-3S | | | | | | | | | | |
| T790M- | 18 | G719A T790M | 60 | F | N | N/A | Adenocarcinoma | N | Osimertinib | IV |
| like-3S | | | | | | | | | | |
| T790M- | 19 | I744_E749deli | 60 | F | N | N/A | Adenocarcinoma | N | Osimertinib | IV |
| like-3S | | nsMKK | | | | | | | | |

All of the methods disclosed and claimed herein can be made and executed without undue experimentation in light of the present disclosure. While the compositions and methods of this disclosure have been described in terms of certain embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1 Russo, A. et al. Heterogeneous Responses to Epidermal Growth Factor Receptor (EGFR) Tyrosine Kinase Inhibitors (TKIs) in Patients with Uncommon EGFR Mutations: New Insights and Future Perspectives in this Complex Clinical Scenario. Int J Mol Sci 20, doi:10.3390/ijms20061431 (2019).
2 Kobayashi, Y. et al. EGFR Exon 18 Mutations in Lung Cancer: Molecular Predictors of Augmented Sensitivity to Afatinib or Neratinib as Compared with First- or Third-Generation TKIs. Clin Cancer Res 21, 5305-5313, doi:10.1158/1078-0432.CCR-15-1046 (2015).
3 Kobayashi, Y. & Mitsudomi, T. Not all epidermal growth factor receptor mutations in lung cancer are created equal: Perspectives for individualized treatment strategy. Cancer Sci 107, 1179-1186, doi:10.1111/cas.12996 (2016).
4 Klughammer, B. et al. Examining Treatment Outcomes with Erlotinib in Patients with Advanced Non-Small Cell Lung Cancer Whose Tumors Harbor Uncommon EGFR Mutations. J Thorac Oncol 11, 545-555, doi:10.1016/j.jtho.2015.12.107 (2016).
5 Rosell, R. et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol 13, 239-246, doi:10.1016/S1470-2045(11)70393-X (2012).
6 Sequist, L. V. et al. Phase III study of afatinib or cisplatin plus pemetrexed in patients with metastatic lung adenocarcinoma with EGFR mutations. J Clin Oncol 31, 3327-3334, doi:10.1200/JCO.2012.44.2806 (2013).
7 Soria, J. C. et al. Osimertinib in Untreated EGFR-Mutated Advanced Non-Small-Cell Lung Cancer. N Engl J Med 378, 113-125, doi:10.1056/NEJMoa1713137 (2018).
8 Scott, L. J. Osimertinib as first-line therapy in advanced NSCLC: a profile of its use. Drugs Ther Perspect 34, 351-357, doi:10.1007/s40267-018-0536-9 (2018).
9 Ramalingam, S. S. et al. Overall Survival with Osimertinib in Untreated, EGFR-Mutated Advanced NSCLC. N Engl J Med 382, 41-50, doi:10.1056/NEJMoa1913662 (2020).
10 Yoshikawa, S. et al. Structural basis for the altered drug sensitivities of non-small cell lung cancer-associated mutants of human epidermal growth factor receptor. Oncogene 32, 27-38, doi:10.1038/onc.2012.21 (2013).
11 Improta, G. et al. Uncommon frame-shift exon 19 EGFR mutations are sensitive to EGFR tyrosine kinase inhibitors in non-small cell lung carcinoma. Med Oncol 35, 28, doi:10.1007/s12032-018-1078-7 (2018).
12 Massarelli, E., Johnson, F. M., Erickson, H. S., Wistuba, II & Papadimitrakopoulou, V. Uncommon epidermal growth factor receptor mutations in non-small cell lung cancer and their mechanisms of EGFR tyrosine kinase inhibitors sensitivity and resistance. Lung Cancer 80, 235-241, doi:10.1016/j.lungcan.2013.01.018 (2013).
13 Yasuda, H., Kobayashi, S. & Costa, D. B. EGFR exon 20 insertion mutations in non-small-cell lung cancer: preclinical data and clinical implications. Lancet Oncol 13, e23-31, doi:10.1016/S1470-2045(11)70129-2 (2012).
14 Arcila, M. E. et al. EGFR exon 20 insertion mutations in lung adenocarcinomas: prevalence, molecular heterogeneity, and clinicopathologic characteristics. Mol Cancer Ther 12, 220-229, doi:10.1158/1535-7163.MCT-12-0620 (2013).
15 Kosaka, T. et al. Response heterogeneity of EGFR and HER2 exon 20 insertions to covalent EGFR and HER2 inhibitors. Cancer Res, doi:10.1158/0008-5472.CAN-16-3404 (2017).
16 Cho, J. H. et al. Osimertinib for Patients With Non-Small-Cell Lung Cancer Harboring Uncommon EGFR Mutations: A Multicenter, Open-Label, Phase II Trial (KCSG-LU15-09). J Clin Oncol 38, 488-495, doi:10.1200/JCO.19.00931 (2020).
17 Callegari, D. et al. L718Q mutant EGFR escapes covalent inhibition by stabilizing a non-reactive conformation of the lung cancer drug osimertinib. Chem Sci 9, 2740-2749, doi:10.1039/c7sc04761d (2018).
18 Bersanelli, M. et al. L718Q Mutation as New Mechanism of Acquired Resistance to AZD9291 in EGFR-Mutated NSCLC. J Thorac Oncol 11, e121-123, doi:10.1016/j.jtho.2016.05.019 (2016).
19 Ercan, D. et al. EGFR Mutations and Resistance to Irreversible Pyrimidine-Based EGFR Inhibitors. Clin Cancer Res 21, 3913-3923, doi:10.1158/1078-0432.CCR-14-2789 (2015).
20 Brown, B. P. et al. On-target Resistance to the Mutant-Selective EGFR Inhibitor Osimertinib Can Develop in an Allele-Specific Manner Dependent on the Original EGFR-Activating Mutation. Clin Cancer Res 25, 3341-3351, doi:10.1158/1078-0432.CCR-18-3829 (2019).
21 Fassunke, J. et al. Overcoming EGFR(G724S)-mediated osimertinib resistance through unique binding characteristics of second-generation EGFR inhibitors. Nat Commun 9, 4655, doi:10.1038/s41467-018-07078-0 (2018).
22 Oztan, A. et al. Emergence of EGFR G724S mutation in EGFR-mutant lung adenocarcinoma post progression on osimertinib. Lung Cancer 111, 84-87, doi:10.1016/j.lungcan.2017.07.002 (2017).
23 Le, X. et al. Landscape of EGFR-dependent and -independent resistance mechanisms to osimertinib and continuation therapy post-progression in EGFR-mutant NSCLC. Clin Cancer Res, doi:10.1158/1078-0432.CCR-18-1542 (2018).
24 Ou, S. I. et al. Emergence of novel and dominant acquired EGFR solvent-front mutations at Gly796 (G796S/R) together with C797S/R and L792F/H mutations in one EGFR (L858R/T790M) NSCLC patient who progressed on osimertinib. Lung Cancer 108, 228-231, doi:10.1016/j.lungcan.2017.04.003 (2017).
25 Kobayashi, Y. et al. Characterization of EGFR T790M, L792F, and C797S Mutations as Mechanisms of Acquired Resistance to Afatinib in Lung Cancer. Mol Cancer Ther 16, 357-364, doi:10.1158/1535-7163.MCT-16-0407 (2017).
26 Oxnard, G. R. et al. Assessment of Resistance Mechanisms and Clinical Implications in Patients With EGFR T790M-Positive Lung Cancer and Acquired Resistance to Osimertinib. JAMA Oncol 4, 1527-1534, doi:10.1001/jamaoncol.2018.2969 (2018).
27 Piotrowska, Z. et al. Landscape of Acquired Resistance to Osimertinib in EGFR-Mutant NSCLC and Clinical Validation of Combined EGFR and RET Inhibition with Osimertinib and BLU-667 for Acquired RET Fusion. Cancer Discov 8, 1529-1539, doi:10.1158/2159-8290.CD-18-1022 (2018).
28 Schoenfeld, A. J. et al. Tumor Analyses Reveal Squamous Transformation and Off-Target Alterations As Early Resistance Mechanisms to First-line Osimertinib in EGFR-Mutant Lung Cancer. Clin Cancer Res, doi:10.1158/1078-0432.CCR-19-3563 (2020).
29 Wu, Y. L. et al. Afatinib versus cisplatin plus gemcitabine for first-line treatment of Asian patients with advanced non-small-cell lung cancer harbouring EGFR mutations (LUX-Lung 6): an open-label, randomised phase 3 trial. Lancet Oncol 15, 213-222, doi:10.1016/S1470-2045(13)70604-1 (2014).
30 Kohler, J. & Schuler, M. LUX-Lung 3: redundancy, toxicity or a major step forward? Afatinib as front-line therapy for patients with metastatic EGFR-mutated lung cancer. Future Oncol 10, 533-540, doi:10.2217/fon.14.9 (2014).
31 Yang, J. C. et al. Clinical activity of afatinib in patients with advanced non-small-cell lung cancer harbouring uncommon EGFR mutations: a combined post-hoc analysis of LUX-Lung 2, LUX-Lung 3, and LUX-Lung 6. Lancet Oncol 16, 830-838, doi:10.1016/S1470-2045(15)00026-1 (2015).
32 Yang, J. C. et al. Afatinib versus cisplatin-based chemotherapy for EGFR mutation-positive lung adenocarcinoma (LUX-Lung 3 and LUX-Lung 6): analysis of overall survival data from two randomised, phase 3 trials. Lancet Oncol 16, 141-151, doi:10.1016/S1470-2045(14)71173-8 (2015).
33 Masood, A., Kancha, R. K. & Subramanian, J. Epidermal growth factor receptor (EGFR) tyrosine kinase inhibitors in non-small cell lung cancer harboring uncommon EGFR mutations: Focus on afatinib. Semin Oncol 46, 271-283, doi:10.1053/j.seminoncol.2019.08.004 (2019).
34 Shi, J. et al. Uncommon EGFR mutations in a cohort of Chinese NSCLC patients and outcomes of first-line EGFR-TKIs and platinum-based chemotherapy. Chin J Cancer Res 29, 543-552, doi:10.21147/j.issn.1000-9604.2017.06.09 (2017).
35 Zhang, Y. et al. Clinical characteristics and response to tyrosine kinase inhibitors of patients with non-small cell lung cancer harboring uncommon epidermal growth factor receptor mutations. Chin J Cancer Res 29, 18-24, doi:10.21147/j.issn.1000-9604.2017.01.03 (2017).
36 Xu, J. et al. EGFR tyrosine kinase inhibitor (TKI) in patients with advanced non-small cell lung cancer (NSCLC) harboring uncommon EGFR mutations: A real-world study in China. Lung Cancer 96, 87-92, doi:10.1016/j.lungcan.2016.01.018 (2016).
37 Yu, H. A. et al. Acquired Resistance of EGFR-Mutant Lung Cancer to a T790M-Specific EGFR Inhibitor: Emergence of a Third Mutation (C797S) in the EGFR Tyrosine Kinase Domain. JAMA Oncol 1, 982-984, doi:10.1001/jamaoncol.2015.1066 (2015).
38 Thress, K. S. et al. Acquired EGFR C797S mutation mediates resistance to AZD9291 in non-small cell lung cancer harboring EGFR T790M. Nat Med 21, 560-562, doi:10.1038/nm.3854 (2015).
39 Uncommon EGFR Mutations Database, (2020).
40 Yang, J. C. et al. Afatinib for the Treatment of NSCLC Harboring Uncommon EGFR Mutations: A Database of 693 Cases. J Thorac Oncol 15, 803-815, doi:10.1016/j.jtho.2019.12.126 (2020).
41 Robichaux, J. P. et al. Mechanisms and clinical activity of an EGFR and HER2 exon 20-selective kinase inhibitor in non-small cell lung cancer. Nat Med 24, 638-646, doi:10.1038/s41591-018-0007-9 (2018).
42 Robichaux, J. P. et al. PanCancer Cell 36, 444-457 e447, doi:10.1016/j.ccell.2019.09.001 (2019).
43 Jacobson, M. P. et al. A hierarchical approach to all-atom protein loop prediction. Proteins—Structure Function and Bioinformatics 55, 351-367, doi:10.1002/prot.10613 (2004).
44 Boyd, S. Molecular operating environment. Chemistry World 2, 66-66 (2005).
45 Galili, T., O'Callaghan, A., Sidi, J. & Sievert, C. heatmaply: an R package for creating interactive cluster heatmaps for online publishing. Bioinformatics 34, 1600-1602, doi:10.1093/bioinformatics/btx657 (2018).
46 Chen, Y. et al. Tumor characteristics associated with engraftment of patient-derived non-small cell lung cancer xenografts in immunocompromised mice. Cancer 125, 3738-3748, doi:10.1002/cncr.32366 (2019).

1-116. (canceled)

117. A method for classifying a cancer sample, the method comprising:
(a) detecting an EGFR mutation in the cancer sample; and
(b) classifying the cancer sample as:
(i) a classical-like EGFR mutant cancer, wherein the EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M;
(ii) a T790M-like-3S EGFR mutant cancer, wherein the EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M;
(iii) a T790M-like-3R EGFR mutant cancer, wherein the EGFR mutation is Ex19del T790M C797S, Ex19del T790M L792H, G724S T790M, L718Q T790M, L858R T790M C797S, or L858R T790M L718Q;
(iv) an Exon20ins-NL EGFR mutant cancer, wherein the EGFR mutation is A767_V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP;
(v) an Exon20ins-FL EGFR mutant cancer, wherein the EGFR mutation is H773_V774 insNPH, H773_V774 insAH, H773dupH, V774_C775 insHV, V774_C775 insPR; or
(vi) a P-loop αC-helix compressing EGFR mutant cancer, wherein the EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747_S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774.

118-186. (canceled)

187. A method for treating a subject for cancer, the method comprising administering an effective amount of an EGFR inhibitor to a subject determined, from analysis of tumor DNA from the subject, to have:
i) a classical-like EGFR mutation;
ii) a T790M-like-3S EGFR mutation
iii) a exon 20 near-loop insertion EGFR mutation;
iv) a exon 20 far-loop insertion EGFR mutation
v) a P-loop αC-helix compressing EGFR mutation.

188. The method of claim 187, wherein the EGFR inhibitor is a first-generation EGFR inhibitor, a second-generation EGFR inhibitor, a third generation EGFR inhibitor, or an EGFR inhibitor specific to mutations associated with EGFR exon 20.

189. The method of claim 188, wherein the EGFR inhibitor is Erlotinib, Geftinib, AZD3759, Sapatinib, Lapatinib, Tucatinib, icotinib, Afatinib, Dacomitinib, Neratinib, Tarlox-TKI, Tarloxotinib, BDTX189, sutetinib, Osimertinib, Nazartinib, Olmutinib, Rocelitinib, Naquotinib, Lazertinib, WZ4002, almonertinib, furmonertinib, abivertinib, alflutinib, mavelertinib, abivertinib, olafertinib, rezivertinib, TAS 6417, AZ5104, TAK-788 (mobocertinib), or DZD9008.

190. The method of claim 187, wherein:
i) the classical-like EGFR mutation is A702T, A763insFQEA, A763insLQEA, D761N, E709A L858R, E709K L858R, E746_A750del A647T, E746_A750del L41W, E746_A750del R451H, Ex19del E746_A750del, K754E, L747_E749del A750P, L747_T751del L861Q, L833F, L833V, L858R, L858R A289V, L858R E709V, L858R L833F, L858R P100T, L858R P848L, L858R R108K, L858R R324H, L858R R324L, L858R S784F, L858R S784Y, L858R T725M, L858R V834L, L861Q, L861R, S720P, S784F, S811F, or T725M;
ii) the T790M-like-3S EGFR mutation is Ex19del T790M, Ex19del T790M L718V, Ex19del T790M G724S, G719A T790M, G719S T790M, H773R T790M, I744_E749del insMKK, L747_K754 delinsATSPE, L858R T790M L792H, L858R T790M V843I, L858R T790M, S768I T790M, or T790M;
iii) the exon 20 near-loop insertion EGFR mutation is A767 V769dupASV, A767_S768insTLA, S768_D770dupSVD, S768_D770dupSVD L858Q, S768_D770dupSVD R958H, S768_D770dupSVD V769M, V769_D770insASV, V769_D770insGSV, V769_D770insGVV, V769_D770insMASVD, D770_N771insNPG, D770_N771insSVD, D770del insGY, D770_N771 insG, D770_N771 insY H773Y, N771dupN, N771dupN G724S, N771_P772insHH, N771_P772insSVDNR, or P772_H773insDNP;
iv) the exon 20 far-loop insertion EGFR mutation is H773_V774 insNPH, H773_V774 insAH, H773dupH, V774_C775 insHV, V774_C775 insPR;
v) the P-loop αC-helix compressing EGFR mutation is A750_I759del insPN, E709_T710del insD, E709A, E709A G719A, E709A G719S, E709K, E709K G719S, E736K, E746_A750del A647T, E746_A750del R675W, E746_T751del insV S768C, Ex19del C797S, Ex19del G796S, Ex19del L792H, Ex19del T854I, G719A, G719A D761Y, G719A L861Q, G719A R776C, G719A S768I, G719C S768I, G719S, G719S L861Q, G719S S768I, G724S, G724S Ex19del, G724S L858R, G779F, I740dupIPVAK, K757M L858R, K757R, L718Q, Ex19del, L718Q L858R, L718V, L718V L858R, L747 S752del A755D, L747P, L747S, L747S L858R, L747S V774M, L858R C797S, L858R L792H, L858R T854S, N771G, R776C, R776H, E709_T710del insD S22R, S752_I759del V769M, S768I, S768I L858R, S768I L861Q, S768I V769L, S768I V774M, T751_I759 delinsN, V769L, V769M, or V774M.

191. The method of claim 187, wherein the subject has lung cancer.

192. The method of claim 191, wherein the subject has non-small cell lung cancer.

193-195. (canceled)

196. The method of claim 187, wherein the EGFR inhibitor is not a first-generation EGFR inhibitor, a second-generation EGFR inhibitor, or a third-generation EGFR inhibitor.

197. The method of claim 196, wherein the EGFR inhibitor is not Erlotinib, Geftinib, AZD3759, Sapatinib, Lapatinib, Tucatinib, icotinib, Afatinib, Dacomitinib, Neratinib, Tarlox-TKI, Tarloxotinib, BDTX189, sutetinib, Osimertinib, Nazartinib, Olmutinib, Rocelitinib, Naquotinib, Lazertinib, WZ4002, almonertinib, furmonertinib, abivertinib, alflutinib, mavelertinib, abivertinib, olafertinib, or rezivertinib.

198-200. (canceled)

201. A method for treating a subject for cancer, the method comprising administering an effective amount of a tyrosine kinase inhibitor to a subject determined, from analysis of tumor DNA from the subject, to have a T790M-like-3R EGFR mutation, wherein the tyrosine kinase inhibitor is not an EGFR inhibitor.

202. The method of claim 201, wherein the tyrosine kinase inhibitor is a PKC inhibitor.

203. The method of claim 202, wherein the PKC inhibitor is Ruboxistaurin, Midostaurin, Sotrastaurin, Chelerythrine, Miyabenol C, Myricitrin, Gossypol, Verbascoside, BIM-1, Bryostatin 1, or Tamoxifen.

204. The method of claim 201, wherein the tyrosine kinase inhibitor is an ALK inhibitor.

205. The method of claim 204, wherein the ALK inhibitor is AZD3463, Brigatinib, Crizotinib, Ceritinib, Alectinib, Lorlatinib, Ensartinib, Entrectinib, Repotrectinib, Belizatinib, Alkotinib, Foritinib, CEP-37440, TQ-B3139, PLB1003, TPX-0131, or ASP-3026.

206. The method of claim 201, wherein the T790M-like-3R EGFR mutation is Ex19del T790M C797S, Ex19del T790M L792H, G724S T790M, L718Q T790M, L858R T790M C797S, or L858R T790M L718Q.

207. The method of claim 201, wherein the subject has lung cancer.

208. The method of claim 207, wherein the subject has non-small cell lung cancer.

209-232. (canceled)
